(19) United States
(12) Patent Application Publication
(10) Pub. No.: US 2024/0393323 A1
(43) Pub. Date: Nov. 28, 2024

(54) Microfluidic Magnetic Separation Device with A Magnetophoretic Gradient for Isolation of Target Cell Populations from Fluid Samples

(71) Applicant: The Board of Trustees of the Leland Stanford Junior University, Stanford, CA (US)

(72) Inventors: Sindy K. Y. Tang, Stanford, CA (US); Nicolas Castano, Redwood City, CA (US); Justin Myles, Redwood City, CA (US)

(21) Appl. No.: 18/693,432

(22) PCT Filed: Sep. 20, 2022
(86) PCT No.: PCT/US2022/044138

§ 371 Date: Mar. 19, 2024

Related U.S. Application Data
(60) Provisional application No. 63/246,069, filed on Sep. 20, 2021.

(51) Int. Cl. G01N 33/543; B03C 1/033; B03C 5/00

(52) U.S. Cl. CPC G01N 33/54326; B03C 1/0332; B03C 5/005; B03C 2201/18; B03C 2201/22; B03C 2201/26

(57) ABSTRACT

Devices, methods, and kits are provided for isolating a target cell from a fluid sample. In particular, a magnetophoretic separation device is provided that applies varying magnetic field strength to flowing magnetically tagged cells. Immunomagnetic negative selection of target cells is used to maintain target cells in their native, unlabeled state. The magnetophoretic separation device is suitable for isolating cells from low volumes of whole blood, which provides an advantage over in-bulk methods that require larger starting volumes of blood. A computer implemented method is also provided for producing target magnetophoretic profiles along the path through which cells travel through a fluidic conduit in the device that is adaptable to a variety of form factors.

### STATEMENT REGARDING FEDERALLY SPONSORED RESEARCH OR DEVELOPMENT

This invention was made with Government support under contracts AI149277 and EB030643 awarded by the National Institutes of Health. The Government has certain rights in the invention.

### BACKGROUND

Basophils are the rarest type of granulocytes in peripheral blood, comprising 1% or less of all leukocytes. Despite their low abundance, basophils are important regulatory and effector cells in a wide range of immune functions and disorders (Galli et al., Curr. Opin. Immunol. 3, 865-872 (1991); Karasuyama et al., J. Allergy Clin. Immunol. 142, 370-380 (2018); Steiner et al., Biomed Res. Int. 2016, U.S. Pat. No. 8,232,830 (2016); and Miyake & Karasuyama, Allergol Int 66, 382-391 (2017)). In particular, basophils play critical roles in allergic diseases. When triggered by stimulants such as allergens, basophils can secrete a range of mediators (e.g., histamine, leukotrienes, platelet activating factor) (Chirumbolo, Blood Transfus. 10, 148-164 (2012); Miyake et al., Allergy 76 (6), 1693-1706 (2021); Borriello & Marone, Handb Exp Pharmacol 241, 121-139 (2017)). They can infiltrate tissues undergoing atopic hypersensitive inflammation (Yamanishi et al., Allergy 75, 2613-2622 (2020)), and act as initiators of allergic inflammation (Miyake et al. (2017), supra; Chirumbolo et al. (2012), supra; Miyake et al. (2021), supra; Siracusa et al., J. Allergy Clin. Immunol. 132, 789-801; quiz 788 (2013)) and possibly of anaphylactic reactions (Korosec et al., Clin. Exp. Allergy 48, 502-512 (2018); Lin et al., Int. Immunol. 32, 213-219 (2020); Korosec et al., J. Allergy Clin. Immunol. 140, 750-758.e15 (2017); Yamaga et al., Allergol Int 69, 78-83 (2020)). Basophils are also involved in other diseases (e.g., enhanced innate immune response against sepsis (Piliponsky et al., Nat. Immunol. 20, 129-140 (2019)), production of angiogenic factors in cancer tumorigenesis (Marone et al., Front. Immunol. 11, 2103 (2020)), modulating humoral response against SARS-CoV-2 (Rodriguez et al., Cell Rep. Med. 1, 100078 (2020)) and immunoregulatory functions (e.g., alveolar macrophage imprinting (Cohen et al., Cell 175, 1031-1044.e18 (2018)), promotion of Th2 cell differentiation (Yamanishi et al., Immunol. Rev. 278, 237-245 (2017); Sokol et al., Nat. Immunol. 10, 713-720 (2009)), recruitment of eosinophils (Nakashima et al., J. Allergy Clin. Immunol. 134, 100-107 (2014); Cheng et al., J. Exp. Med. 212, 513-524 (2015)).

The identification and isolation of basophils is important for both clinical and research studies. For example, the activation of basophils in the presence of allergens ex vivo has been found to correlate with the allergic status of the subject. The basophil activation test (BAT), an ex vivo blood test, has demonstrated high specificity (75-100%) and sensitivity (77-98%) in the diagnosis of allergy towards a range of allergens including peanut, cow's milk, egg, therapeutic drugs, and pollen (Duan et al., Allergy 76 (6), 1800-1812 (2020); Hoffmann et al., Allergy 70, 1393-1405 (2015); Hemmings et al., Curr. Allergy Asthma Rep. 18, 77 (2018); Ozdemir et al., Am J Rhinol Allergy 25, e225-31 (2011); Steiner et al., Front. Pharmacol. 7, 171 (2016)). Here, basophils are typically identified by gating schemes in flow cytometry data, commonly as SSC^{low}/HLA-DR⁻/CD123⁺. Basophil activation is then measured by the level of expression of activation markers CD63, CD203c, and/or avidin (Lotzsch et al., Clin. Transl. Allergy 6, 45 (2016); MacGlashan et al., Clin. Exp. Allergy 40, 1365-1377 (2010); Mukai et al., J. Allergy Clin. Immunol. 139, 889-899.e11 (2017)). In fundamental research, bulk and single-cell RNA sequencing of basophils has uncovered mechanistic pathways and transcriptional profiles associated with basophil differentiation (Hamey et al., Allergy 76, 1731-1742 (2021); Sasaki et al., Allergol Int 65, 127-134 (2016)) and function (Miyake et al. (2021), supra; Marone et al., Front. Immunol. 11, 2103 (2020); Cohen et al., Cell 175, 1031-1044.e18 (2018); Winter et al., J. Allergy Clin. Immunol. 148 (2), 428-438 (2021); Chhiba, et al., J. Immunol. 198, 4868-4878 (2017)). The accuracy of RNA-sequencing, especially deep transcriptional profiling of specific cell types, rely on starting with purified target cell populations commonly obtained from cell lines or by fluorescence-activated cell sorting (FACS) (Hwang et al., Exp Mol Med 50, 1-14 (2018); Amamoto et al. Elife 8, e51452 (2019); Nguyen, et al., Front. Cell Dev. Biol. 6, 108 (2018)).

Since basophils are rare, their isolation from whole blood has been challenging.

Conventional methods of separation are based on density gradient, FACS, immunomagnetic negative selection, or a combination of these methods. Shiono et al. demonstrated a flow-through basophil isolation method using 5 Percoll density gradients, but their approach suffered from relatively low recovery (˜56-77%) and modest purity (˜51-72%) (Shiono et al., J Sep Sci 39, 3062-3071 (2016)). Degenehart et al. (J. Allergy Clin. Immunol. 82, 455-461 (1988)) used FACS to isolate viable basophils from white blood cells (WBCs) enriched from venous blood with a mean purity of 84% (range 75-95%) and a recovery of 20% (range 15-30%). This method required upstream removal of red blood cells (RBCs) by density gradient or lysis. The need for surface markers may also affect downstream assays that require antibody labeling. The best performing basophil isolation methods reported thus far use immunomagnetic negative selection of basophils (Gibbs & Falcone, Basophils and mast cells: methods and protocols. 2163, 35-48 (Springer US, 2020)). Using a commercial immunomagnetic negative selection kit (EasySep™ Human Basophil Isolation Kit, STEMCELL Technologies, <stemcell.com/easysep-human-basophil-isolation-kit.html>), basophils were isolated with high purity (mean purity of 99.3%, range 97-100%) from WBCs, where RBCs were first removed using an RBC aggregation agent followed by centrifugation. Nevertheless, this method has several drawbacks: 1) The recovery was modest, with a mean recovery of 75.6% and a large variability in performance (range 39-100%) (Gibbs et al., Clin. Exp. Allergy 38, 480-485 (2008)). 2) It required a large volume (30 mL) of blood. 3) It required many manual pipetting and centrifugation steps. Although this procedure was faster than approaches that use multiple Percoll gradients combined with immunomagnetic negative selection (which took 4 hours) (MacGlashan, PLOS One 10, e0126435 (2015)), this process still required about 90 minutes (Gibbs et al. (2008), supra; Tsang et al., J. Immunol. Methods 233, 13-20 (2000)).

Thus, there remains a need for better, more efficient methods of isolating cells, particularly rare cell types.

### SUMMARY

Devices, methods, and kits are provided for isolating target cells from a fluid sample. Microfluidics has emerged as a promising technology for the precise separation and isolation of cells. Negative selection magnetic-activated cell sorting (MACS) is a popular, high-fidelity means for isolating target cell populations using antibodies and magnetic particles to remove non-target cells in a magnetic field. Current methods lack a streamlined approach for applying magnetophoretic forces that are compatible with downstream microfluidic process flows. Disclosed herein is a magnetophoretic separation device (MSD), that applies varying magnetic field strength to flowing magnetically tagged cells. Immunomagnetic negative selection of target cells is used to maintain target cells in their native, unlabeled state. The MSD is capable of isolating cells from low volumes of whole blood, which provides an advantage over in-bulk methods that require larger starting volumes of blood. On-chip microfluidic MACS methods generally require the integration of multiple inlets/outlets, and require a precise alignment with off-chip magnets; otherwise, the isolation purity is compromised. In certain embodiments, the device, described herein, does not require careful alignment and is reusable, with its only consumable being tubing through which the cell suspension flows.

In one aspect, a magnetophoretic separation device (MSD) is provided, the MSD comprising: a) a single magnet or a magnetic array comprising a plurality of magnets; b) a scaffold comprising a slot to secure the single magnet or magnetic array and one or more channels, wherein each channel comprises one or more fluidic conduits connected to one or more outlets; and c) one or more magnetic flux concentrators in proximity to the one or more fluidic conduits, wherein spacing of the one or more magnetic flux concentrators relative to the single magnet or magnetic array and the one or more fluidic conduits is selected to produce a target magnetophoretic gradient profile, wherein magnetophoretic force varies along the length of the one or more fluidic conduits.

In certain embodiments, the magnetic array is a Halbach array. For example, the Halbach array may be a cylindrical Halbach array comprising a plurality of ring magnets or a linear Halbach array comprising a plurality of bar magnets.

In certain embodiments, the magnetic array comprises multiple magnets with opposing polarities, multiple magnets with aligned polarities, a grid of magnets with alternating polarities, or self-assembling magnetic colloids.

In certain embodiments, the magnetic array comprises at least five magnets.

In certain embodiments, the magnets are permanent magnets or electromagnets. In some embodiments, the permanent magnets are neodymium magnets.

In certain embodiments, a magnetic flux concentrator comprises a plurality of ferromagnetic wires, sheets, self-assembling colloids, or other configuration of ferromagnetic material running alongside a fluidic conduit, wherein spacing of the wires, sheets, self-assembling colloids, or other configuration of ferromagnetic material relative to the single magnet or magnetic array and the fluidic conduit is selected to produce a desired target magnetophoretic gradient profile.

In certain embodiments, a fluidic conduit is in the form of a fluidic tubing positioned within a scaffold that defines its position relative to the single magnet or magnetic array and magnetic flux concentrators. In some embodiments, the fluidic tubing has an inner diameter sufficiently small that magnetic flux from the magnetic flux concentrator reaches the center axis of the fluidic tubing. In some embodiments, the inner diameter is sufficiently large to retain captured non-target cells without obstructing target cells from flowing through the fluidic tubing.

In certain embodiments, a fluidic conduit is machined, embossed, rastered, etched or molded into materials such as acrylic, poly(methyl methacrylate) (PMMA), silicon, or polydimethylsiloxane (PMDS). In certain embodiments, the MSD is designed using a processor programmed to model magnetic field and magnetophoretic forces at different locations relative to the single magnet or magnetic array and identify a set of spatial coordinates to position a fluidic conduit and a magnetic flux concentrator relative to the magnets to achieve a target exponential magnetophoretic force profile along the fluidic conduit.

In certain embodiments, a channel is planar, multi-layered, or three-dimensional.

In certain embodiments, a fluidic conduit provides a straight fluid path, a curved fluid path, or a serpentine fluid path. In some embodiments, the fluid path revolves around a plurality of ring magnets. In some embodiments, the fluidic path runs parallel to a plurality of bar magnets. In some embodiments, the fluidic path is bifurcated to direct fluid flow to regions of concentrated magnetic flux.

In certain embodiments, the channel width ranges from 0.01 mm to 100 mm, including any channel width within this range such as 0.01 mm, 0.05 mm, 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, or 100 mm.

In certain embodiments, the channel height ranges from 0.01 mm to 100 mm, including any channel height within this range such as 0.01 mm, 0.05 mm, 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, or 100 mm.

In certain embodiments, the channel length ranges from 1 mm to 1000 mm, including any channel height within this range such as 1 mm, 5 mm, 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, 100 mm, 120 mm, 140 mm, 160 mm, 180 mm, 200 mm, 220 mm, 240 mm, 260 mm, 280 mm, 300 mm, 320 mm, 340 mm, 360 mm, 380 mm, 400 mm, 420 mm, 440 mm, 460 mm, 480 mm, 500 mm, 520 mm, 540 mm, 560 mm, 580 mm, 600 mm, 620 mm, 640 mm, 660 mm, 680 mm, 700 mm, 720 mm, 740 mm, 760 mm, 780 mm, 800 mm, 820 mm, 840 mm, 860 mm, 880 mm, 900 mm, 920 mm, 940 mm, 960 mm, 980 mm, or 1000 mm.

In certain embodiments, the fluid passes through the MSD in one or more passes.

In certain embodiments, a fluidic conduit has a square, rectangular, circular, elliptical, or polygonal cross-sectional shape.

In certain embodiments, a channel further comprises a fluidic element that focuses all or a subset of cells from a fluid sample towards an area of higher magnetic force field. In some embodiments, the fluidic element is a constriction, an expansion, a vane, or a sheath flow device.

In certain embodiments, the MSD further comprises a gutter connected to the fluidic conduit.

In certain embodiments, the MSD may mix the fluid within a fluidic conduit using a staggered herringbone mixer, dean vortices, parallel lamination of fluidic streamlines or other microstructures such as micropillars and steps.

In certain embodiments, the magnetic array with or without a magnetic flux concentrator produces a magnetic flux density ranging from 0.1 tesla (T) to 10 T, including any magnetic flux density within this range such as 0.1 T, 0.15 T, 0.2 T, 0.25 T, 0.3 T, 0.35 T, 0.4 T, 0.45 T, 0.5 T, 0.55 T, 0.6 T, 0.65 T, 0.7 T, 0.75 T. 0.8 T, 0.85 T. 0.9 T, 0.95 T, or 10 T.

In certain embodiments, a magnetic flux concentrator has a relative magnetic permeability ranging from 100 to 100,000, including any relative magnetic permeability in this range such as 100, 200, 300, 400, 500, 600, 700, 800, 900, 1000, 1500, 2000, 2500, 3000, 3500, 4000, 4500, 5000, 5500, 6000, 6500, 7000, 8000, 8500, 9000, 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, or 100,000.

In certain embodiments, a magnetic flux concentrator comprises a fluidic channel comprising a ferrofluid or a self-assembled magnetic colloid.

In certain embodiments, a magnetic flux concentrator radius can be adjusted to tune peak magnitude of the magnetic force on cells in the fluidic conduit.

In certain embodiments, the magnetophoretic force increases exponentially along the length of the fluidic conduit.

In certain embodiments, the MSD further comprises two or more channels, each channel comprising a separate fluidic conduit connected to a different outlet, wherein each channel can be used to separate target cells from non-target cells from a different fluid sample in parallel. In some embodiments, each outlet is connected to a separate collection reservoir or a common collection reservoir. In some embodiments, each channel or a subset of channels shares the same magnetic array and magnetic flux concentrator, or each channel has a different magnetic array and magnetic flux concentrator.

In certain embodiments, the MSD further comprises a plurality of pumps. In some embodiments, the pumps are syringe pumps, diaphragm pumps, peristaltic pumps or piston pumps, or any combination thereof. In some embodiments, the pumps are positive displacement pumps or pressure generating pumps.

In certain embodiments, the MSD further comprises a plurality of sensors that monitor flow rate or pressure, or a combination thereof. In some embodiments, the pumps and sensors are configured to maintain the flow rate through the magnetic separation device in a range from 0.01 mL/hr to 20,000 L/hr.

In certain embodiments, the MSD comprises: a) a Halbach array comprising a plurality of magnets; b) a scaffold comprising a slot to secure the Halbach array and a passage tunnel to position a fluidic tubing; and c) a magnetic flux concentrator comprising a plurality of wires running parallel to fluidic tubing, wherein spacing of the wires relative to the Halbach array and the fluidic tubing is selected to produce a target exponential magnetophoretic gradient profile along the path of the fluidic tubing.

In another aspect, a microfluidic magnetic device for isolation of one or more types of target cells from a fluid sample comprising a heterogenous population of cells is provided, the device comprising: a) one or more inlets to receive the fluid sample; b) one or more mixers connected to the one or more inlets, wherein the one or more mixers mix magnetic nanoparticles (MNPs) and one or more negative selection binding agents with the cells, wherein the one or more negative selection binding agents selectively bind to one or more surface markers on non-target cells, wherein the one or more surface markers are not present on the one or more types of target cells; c) one or more incubation tubings connected to the one or more mixers, wherein the cells are incubated with the MNPs and the one or more negative selection binding agents in the one or more incubation tubings, wherein the non-target cells that are bound to the one or more negative selection binding agents are selectively magnetically labeled with the MNPs; d) an MSD described herein, wherein the one or more channels of the MSD are connected to the one or more incubation tubings, wherein the MSD captures the magnetically labeled non-target cells and outputs the one or more types of target cells; and e) one or more outlets connected to the MSD, wherein the one or more types of target cells are expelled from the one or more outlets.

In certain embodiments, the microfluidic magnetic device further comprises one or more deterministic lateral displacement (DLD) channels or curved microchannels that sort cells based on size, wherein each DLD channel or curved microchannel is positioned between one of the inlets and one of the mixers, wherein an input end of each DLD channel or curved microchannel is connected to one of the inlets and an output end of each DLD channel or microchannel is connected to one of the mixers. In some embodiments, the one or more DLD channels and the one or more mixers are contained on a microfluidic chip comprising a first layer comprising the one or more mixers and a second layer comprising the one or more DLD channels. In some embodiments, the first layer comprising the one or more mixers is above the second layer comprising the one or more DLD channels. In some embodiments, the one or more DLD channels further comprise a coating to reduce cell adhesion to the DLD channels. For example, the coating may comprise a non-ionic surfactant.

In certain embodiments, the microfluidic magnetic device further comprises a means for applying a secondary Dean flow velocity field or a hydrophoretic force to the one or more curved microchannels when fluid passes through the curved microchannels. In some embodiments, the one or more curved microchannels vary in width along the length of the curved microchannels. In some embodiments, the one or more curved microchannels further comprise perpendicular or angled steps.

In certain embodiments, the one or more types of target cells comprise an immune cell, a blood cell, a stem cell, or a cancer cell, or any combination thereof. In some embodiments, the one or more target cells comprise immune cells such as, but not limited to, a basophil, a neutrophil, an eosinophil, a mast cell, a monocyte, a dendritic cell, a macrophage, a T cell, a B cell, or a natural killer cell, or any combination thereof.

In certain embodiments, the fluid sample is a blood sample.

In certain embodiments, a DLD channel or curved microchannel separates white blood cells from red blood cells.

In certain embodiments, a mixer comprises a Herringbone-grooved serpentine channel. In some embodiments, the Herringbone-grooved serpentine channel has dimensions of about 200 μm in width and about 70 μm in height. In some embodiments, the Herringbone-grooved serpentine channel comprises a groove having a height of about 30 μm.

In certain embodiments, a negative selection binding agent is an antibody, antibody mimetic, aptamer, or ligand.

In certain embodiments, the microfluidic magnetic device further comprises a flow-through microfluidic cytometer capable of measuring numbers and the properties of the target cell, wherein the flow-through microfluidic cytometer is fluidically connected to the magnetophoretic separation device.

In certain embodiments, the microfluidic magnetic device further comprises a plurality of pumps. In some embodiments, the pumps are syringe pumps, diaphragm pumps, peristaltic pumps or piston pumps, or any combination thereof. In some embodiments, the pumps are positive displacement pumps or pressure generating pumps.

In certain embodiments, the microfluidic magnetic device further comprises a plurality of sensors that monitor flow rate or pressure, or a combination thereof. In some embodiments, the pumps and sensors are configured to maintain the flow rate through the magnetic separation device in a range from 0.01 mL/hr to 20,000 L/hr.

In another aspect, a method of using a microfluidic magnetic device, described herein, is provided, the method comprising: a) introducing the fluid sample comprising the heterogenous population of cells into the one or more inlets; b) mixing the magnetic nanoparticles (MNPs) and the one or more negative selection binding agents with the cells using the one or more mixers, wherein the one or more negative selection binding agents selectively bind to the one or more surface markers on the non-target cells, wherein the one or more surface markers are not present on the one or more types of target cells; c) incubating the cells with the MNPs and the one or more negative selection binding agents in the one or more incubation tubings, wherein the non-target cells that are bound to the one or more negative selection binding agents are selectively magnetically labeled with the MNPs; d) applying a magnetophoretic gradient to capture the magnetically labeled non-target cells from the fluid sample using the MSD; and e) collecting the one or more types of target cells expelled from the one or more outlets.

In certain embodiments, the microfluidic magnetic device comprises one or more deterministic lateral displacement (DLD) channels or a curved microchannels, wherein the method further comprises flowing the fluid sample into the one or more DLD channels or curved microchannels, wherein the one or more DLD channels or curved microchannels separate cells based on size.

In certain embodiments, the fluid sample is a blood sample.

In certain embodiments, a DLD channel or curved microchannel separates white blood cells from red blood cells.

In certain embodiments, the method further comprises separating white blood cells from red blood cells by density gradient centrifugation, wherein said introducing the fluid sample into the inlet comprises introducing a fluid sample comprising the separated white blood cells into the inlet.

In certain embodiments, the method further comprises sorting cells by using an acoustic radiation force or dielectrophoretic activity in an electric field prior to said mixing the nanoparticles (MNPs) and the negative selection binding agent with the cells.

In another aspect, a microfluidic magnetic device for isolation of a target cell from a blood sample is provided, the device comprising: a) one or more inlets to receive a blood sample; b) one or more deterministic lateral displacement (DLD) channels connected to the inlets, wherein the one or more DLD channels separate white blood cells from red blood cells in the blood sample; c) one or more mixers connected to the one or more DLD channels, wherein the DLD channels mix magnetic nanoparticles (MNPs) and one or more negative selection binding agents with the separated white blood cells, wherein the one or more negative selection binding agents selectively bind to one or more surface markers on non-target cells, wherein the one or more surface markers are not present on the one or more types of target cells; d) one or more incubation tubings connected to the one or more mixers, wherein the white blood cells are incubated with the MNPs and the one or more negative selection binding agents in the one or more incubation tubings, wherein the non-target cells that are bound to the one or more negative selection binding agents are selectively magnetically labeled with the MNPs; e) a magnetophoretic separation device (MSD) connected to the one or more incubation tubings, wherein the MSD applies an exponentially increasing magnetophoretic gradient to capture the magnetically labeled non-target cells; and f) one or more outlets connected to the MSD, wherein the one or more types of target cells are expelled from the one or more outlets.

In certain embodiments, a target cell is an activated basophil or an unactivated basophil.

In certain embodiments, a negative selection binding agent is an antibody, antibody mimetic, aptamer, or ligand.

In certain embodiments, the one or more negative selection binding agents comprise an anti-HLA-DR antibody, an anti-CD2 antibody, an anti-CD3 antibody, an anti-CD14 antibody, an anti-CD15 antibody, an anti-CD16 antibody, an anti-CD19 antibody, an anti-CD20 antibody, an anti-CD24 antibody, an anti-CD34 antibody, an anti-CD36 antibody, an anti-CD45RA antibody, an anti-CD56 antibody, an anti-CD66b antibody, and an anti-glycophorin A antibody, or any combination thereof.

In another aspect, a kit comprising an MSD or a microfluidic magnetic device, described herein, and instructions for isolating one or more types of target cells from a fluid sample are provided.

In certain embodiments, the kit further comprises one or more negative selection binding agents that selectively bind to one or more surface markers on non-target cells, wherein the surface markers are not present on the one or more types of target cells. In some embodiments, a negative selection binding agent is an antibody, antibody mimetic, aptamer, or ligand. In some embodiments, a target cell is an immune cell, a blood cell, a stem cell, or a cancer cell. In some embodiments, a target is an immune cell such as, but not limited to, a basophil, a neutrophil, an eosinophil, a mast cell, a monocyte, a dendritic cell, a macrophage, a T cell, a B cell, or a natural killer cell.

In certain embodiments, the kit further comprises magnetic nanoparticles.

In another aspect, a computer implemented method for controlling a microfluidic magnetic device, described herein, is provided, the computer performing steps comprising: a) injecting the MNPs and the negative selection binding agent into the DLD or curved microchannel through the inlet; b) injecting the fluid sample into the DLD or curved microchannel through the inlet; c) adjusting fluidic flow to a target steady rate; d) controlling amount of time the cells are incubated with the MNPs and the negative selection binding agent in the incubation tubing; and e) controlling flow of the cells through the MSD while applying an exponentially increasing magnetophoretic gradient to capture the magnetically labeled non-target cells from the fluid sample using the MSD.

In another aspect, a non-transitory computer-readable medium is provided comprising program instructions that, when executed by a processor in a computer, causes the processor to perform the computer implemented method for controlling the microfluidic magnetic device.

In another aspect, a system for isolating a target cell from a blood sample is provided, the system comprising: a) a microfluidic magnetic device for isolation of a target cell from a blood sample, the device comprising: (i) an inlet to receive a blood sample; (ii) a deterministic lateral displacement (DLD) channel or curved microchannel connected to the inlet, wherein the DLD or curved microchannel separates white blood cells from red blood cells in the blood sample; (iii) a mixer connected to the DLD or curved microchannel, wherein the DLD or curved microchannel mixes magnetic nanoparticles (MNPs) and a negative selection binding agent with the separated white blood cells, wherein the negative selection binding agent selectively binds to a surface marker on non-target cells, wherein the surface marker is not present on the target cell; (iv) an incubation tubing connected to the mixer, wherein the white blood cells are incubated with the MNPs and the negative selection binding agent in the incubation tubing, wherein the non-target cells that are bound to the negative selection binding agent are selectively magnetically labeled with the MNPs; (v) a magnetophoretic separation device (MSD) connected to the incubation tubing, wherein the MSD applies an exponentially increasing magnetophoretic gradient to capture the magnetically labeled non-target cells; and (vi) an outlet connected to the MSD, wherein the target cell is expelled from the outlet; and b) a processor programmed to control the microfluidic magnetic device according to the computer implemented method.

In another aspect, a computer implemented method for determining positioning of a fluidic conduit and a magnetic flux concentrator (MFC) relative to the plurality of magnets of the magnetophoretic separation device (MSD) is provided, the computer performing steps comprising: modeling magnetic field and magnetophoretic forces at different locations relative to a magnetic array; and identifying a set of spatial coordinates to position the fluidic conduit and the magnetic flux concentrator relative to the plurality of magnets to achieve a target exponential magnetophoretic force profile along the fluidic conduit.

In certain embodiments, the computer performs steps comprising: a) generating a target mathematical profile related to the magnetic force field (∇·B̅)B̅ as a function of the path of the fluidic conduit s, wherein the upper bound of the target value for (∇·B̅)B̅ metric is set according to the highest value of (∇·B̅)B̅ when the distances between the fluidic path and magnetic array and between the fluidic path and the MFC are minimal, e.g., for zₚₐₜₕ=1 mm and d_{MFC}=0 mm; b) identifying different combinations of zₚₐₜₕ and d_{MFC} that could generate a target magnetophoretic force profile along the path of the fluidic conduit by plotting (∇·B̅)B̅ obtained from a parametric sweep on a surface as a function of zₚₐₜₕ and d_{MFC}; c) identifying candidate paths (q) on the (∇·B̅)B̅ surface that would generate a target profile f(s); d) translating the candidate parametric paths into physical path positions; e) performing a high-resolution numerical simulation of the magnetic field in 3-dimensional (3D) free space over the magnetic array to identify a set of (x, y, z) points in the simulation that satisfy the following two criteria: 1) d\vec{B}(x,y,z)/dy≈0, and 2) ∥\vec{B}_{3D,yz}(x,y,z)∥≈∥\vec{B}_{2D}(zₚₐₜₕ)∥; f) displaying sets of 3D candidate paths translated from parameter space that achieve the target magnetophoretic force profile; g) evaluating 3D candidate paths using a cost function to select for a path that minimizes the following functions:

$${α}_{1} = max ( \frac{dz}{dx} ) - 1 , \qquad 1 )$$

which selects for paths with gradual slopes for the fluidic conduit to follow in the xz-plane,

$${α}_{2} = {\int }_{0}^{L} {d}_{MFC} dx - \frac{0.45 L}{2} , \qquad 2 )$$

which selects for MFC paths with gradually varying d_{MFC} from 450 to 0 μm across the total length L of the path to facilitate the 3D printing and the threading of the MFC. 3) Σⱼ₌₁^{N}|∥\vec{B}_{2D,j}∥−∥\vec{B}_{3D,yz,j}∥|, which selects for paths that can best match ∥\vec{B}_{3D,yz}(x, y, z)∥ with ∥\vec{B}_{2D}(zₚₐₜₕ)∥ across all N points in the path; and h) selecting the path e with the minimum cost function

$${min}_{θ} \frac{1}{3} {\sum }_{i = 1}^{3} {[ {α}_{i} ( θ ) ]}^{2}$$

as the final position of the fluidic conduit and the MFC to construct the MSD.

In another aspect, a computer implemented method for determining positioning of a fluidic conduit and a magnetic flux concentrator (MFC) relative to the plurality of magnets of the magnetophoretic separation device (MSD) is provided, the computer performing steps comprising: modeling magnetic field and magnetophoretic forces at different locations relative to the magnetic array; and modeling fluidic forces on the cells and the magnetic nanoparticles present within a channel; and identifying a set of spatial coordinates to position the fluidic conduit and the magnetic flux concentrator relative to the plurality of magnets to achieve a target captured cell density along the fluidic conduit.

In certain embodiments, the computer performs steps comprising: a) generating a target mathematical cell capture profile for capture of magnetically labeled non-target cells; b) performing a high-resolution numerical simulation of the magnetic field in 3-dimensional (3D) free space over the magnet array for a variety of values of the MFC dimensions to identify the magnetic force F=η(B·grad(B)) on the magnetically labeled non-target cells relative to the magnet array, where η is a constant which comprises the number of magnetic particles attached to a cell, the volume of the magnetic particles and the difference in susceptibility between the magnetic particles and the fluid; c) performing a high-resolution numerical simulation of the fluid flow in 3-dimensional (3D) free space within the MSD channel to determine the fluidic force on cells for a given flow rate; d) performing particle tracking simulations of cells within the MSD channel based on the sum of both fluidic force (c) and magnetic force (b); and e) using the particle simulations to predict final cell density on the channel wall and iterating or performing an optimization on the MFC position to achieve the desired cell capture profile in (a). In certain embodiments, performing step b) comprises performing said high-resolution numerical simulation using a variety of values of the dimensions of the gap from the MFC to the channel.

In certain embodiments, a fluidic conduit is provided by fluidic tubing positioned relative to a magnetic array or magnetic flux concentrator.

In another aspect, a non-transitory computer-readable medium is provided comprising program instructions that, when executed by a processor in a computer, causes the processor to perform a computer implemented method described herein.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIGS. 1A-1C. Overview of the basophil isolation device (iBID) process flow. (FIG. 1A) Schematic diagram of the microfluidic circuit for WBC enrichment and basophil isolation by magnetophoretic negative selection. All injections and withdrawals from the system were flow rate controlled. Enriched WBCs were then mixed with isolation reagents in a serpentine Herringbone mixer channel before flowing through an incubation tubing with a length set by the Qₒᵤₜ and the target incubation time. Magnetic nanoparticle (MNP)-tagged non-basophils were then sequestered in the MSD and leaving only enriched basophils at the outlet. (FIG. 1B) Image of the microfluidic chip showing inlets/outlets (left); and microscopy images of different stages of the DLD array showing WBC enrichment and of the mixer showing mixing of two fluorescent streams by 20th mixing unit (M20) (right). (FIG. 1C) Schematic diagram and photograph of the magnetic separation device (MSD). The diagram shows the locations of 4 passes of tubing with steel wires acting as MFC running parallel. The image shows brown-tinted fluid entering and clear fluid exiting, qualitatively demonstrating the depletion of MNP in the MSD.

FIGS. 2A-2C. Simulation data used for the design of the magnetic separation device (MSD). (FIG. 2A) Representative simulation data from the parametric sweep for zₚₐₜₕ and d_{MFC} (distance between cell path and magnetic flux concentrators (MFCs)) at a center-plane A-A. The rainbow colormap shows magnetic flux density gradient and the thermal colormap shows the norm of the magnetic flux density. (FIG. 2B) The resulting magnetic force field norm in the tubing across different values of zₚₐₜₕ and d_{MFC}. The colormap represents the magnetic force field norm B·grad(B) and the arrows indicate the direction of the magnetophoretic force. (FIG. 2C) The variation in dead zone (DZ) position, where the net magnetic force field is zero. The origin of (y_{DZ}, z_{dz}) is at the center of the tubing cross section. Inflections in the plot are due to changes in the finite element mesh when the geometry changes as we sweep over different values of zₚₐₜₕ and d_{MFC}. Discontinuities between passes are due to the edges of the magnets when the tubing path turned around to pass over the Halbach array again.

FIGS. 3A-3D. MSD design pipeline. (FIG. 3A) We started with a target magnetophoretic profile across the tubing length which we quantified by the maximum value of (∇·B̅)B̅ in the cross section of the tubing at a given position s. (FIG. 3B) Using the target profile as a guide, we identified all the paths e that would recapitulate the target profile when ascending the parametric surface which we defined with 2D simulations (see FIG. 2). (FIG. 3C) Candidate parametric paths e were translated to physical 3D candidate paths by finding the point at each position in the tubing path that satisfied the criteria: (1) horizontal flux in the yz-plane, and (2) 3D magnetic field norm in the yz-plane matches the 2D magnetic field norm at a given zₚₐₜₕ. (FIG. 3D) The best path was selected to minimize sharp changes in the tubing path (1), promote gradual changes in d_{MFC} (α₂), and minimize error between the 2D and 3D field norms.

FIGS. 4A-4G. Purity and recovery results. (FIG. 4A) Representative flow cytometer data for an experiment characterizing the standalone MSD operated at 6 mL/hr. The dot plots show the initial CD45⁺ cells from which basophils were identified by CD123⁺/CCR3⁺. Purity was calculated as the proportion of basophils in the CD45⁺ gate. Recovery was calculated by dividing the basophil count in the isolated suspension (right column) to the basophil count in the WBC control sample (left column). Basophil purity (FIG. 4B) and recovery (FIG. 4C) in the standalone MSD characterization (n=32 across all flow rates). Basophil purity (FIG. 4E) and recovery (FIG. 4F) compared across different modes of basophil isolation. The MSD mode was operated at 3 mL/hr (n=5). Approximate flow rates in the manual MSD were 6-8.5 mL/hr (n=6). The i-BID was operated at blood injection rates of 3-5 mL/hr (n=9). We conducted n=7 runs for the EasySep™ magnet and n=6 runs for the direct isolation kit. Box and whisker plots presented are Tukey's box plots (see Methods). *P<0.01, **P<0.0001. Spider plots compare the purity, recovery, and time to isolate for (FIG. 4D) the syringe pump-driven MSD, the manual MSD, and the EasySep™ magnet, and for (FIG. 4G) the i-BID and the Direct Isolation Kit.

FIGS. 5A-5D. The levels of basophil activation (as measured by % CD63+) in the i-BID product when subjected to RPMI (negative BAT control) and anti-IgE (positive BAT control) were similar to the levels of basophil activation in whole blood samples that were processed with the standard BAT protocol.²⁹ Representative flow cytometer data for (FIG. 5A) a healthy donor, and (FIG. 5B) an allergic donor, respectively. The levels of basophil activation for (FIG. 5C) heathy donors (n=3), and (FIG. 5D) allergic patients (n=4), respectively.

FIG. 6. Computer-aided design (CAD) models of two versions of the magnetic separation device (MSD). We used Formlab's Grey Pro™ resin for its rigidity. The 3D-printed scaffold surrounded the magnet housing with ample material so that the magnets would not deform the MSD when repelling each other in a Halbach configuration. Both CAD models were used in this work interchangeably exhibiting no difference in performance. They both produced identical magnetic force field profiles with the same tubing path and relative positions between the MFCs and the magnets. The tolerances on the single-piece version (left) allowed for a snug fit around the magnets. The two-piece version (right) ensured the Halbach array could be securely sandwiched using aluminum bolts and nuts, and it facilitated loading and unloading the magnets. For d_{MFC}<300 μm, to circumvent the minimum feature resolution limit of the Form2 printer that we used, we joined the holes to thread the tubing and MFC.

FIG. 7. The gating process used for evaluating purity and recovery. Enriched WBC from DLD channels was split into 100 μL volumes for testing different flow conditions in the MSD. One volume acted as the control to used to set the CD45 gate and determine the expect number of basophils in the MSD product. Following the convention established by STEMCELL for assessing the purity, basophils purity was determined considering only events that were CD45+. Events outside this gate in the MSD samples were due to bubbles or debris entering the flow cytometer.

FIGS. 8A-8C. Variation in the mean deflection angle across the parameter space and the tubing path. (FIG. 8A) All deflection angles of n vectors in the tubing cross-section domain are averaged and weighed by their deflection magnitude to give a weighted mean angle, ζ. Angles greater than −45° (closer to) 0° indicate a horizontally dominated deflection, i.e., along the global z-axis, that is influenced by the magnetic flux concentrators (MFCs) more than by the Halbach array. (FIG. 8B) Heatmap illustrating the range of horizontally deflected (yellow) to vertically deflected (black) configurations as a function of zₚₐₜₕ and d_{MFC}. (FIG. 8C) ζ approximated along the tubing path. We used the known target maximum magnetic force field value and d_{MFC} at position s to look up zₚₐₜₕ, and we used zₚₐₜₕ and d_{MFC} to look up the corresponding ζ from a smooth cubic interpolation of the heatmap in FIG. 8B. The inflection points are attributed to changes in domain discretization when the values of zₚₐₜₕ and d_{MFC} are varied and the model is re-meshed. The discontinuities between passes are attributed to edge effects at the ends of the magnets where the tubing turns to run across the Halbach array again.

FIG. 9. Schematic of microfluidic magnetic separation device with exponential magnetophoretic gradient for cell isolation from whole blood. An MSD comprising a stack of ring magnets is used in a cylindrical Halbach array as a magnetic flux source. A target magnetic force field profile could be defined by wrapping channels and MFCs around the magnet with some varying distance from the magnet.

FIG. 10. The fraction of captured non-basophils across 30 serpentine gutter units positioned along the length of the MSD is a function of flow rates (1,2, and 3 mL/hr rates are shown) and designed spacing between the MFC and the channel.

FIGS. 11A-11B. Schematic representation of one embodiment of an MFC design with a serpentine channel placed adjacent to a magnet assembly consisting of two opposing magnets sandwiches steel magnetic flux concentrator (FIG. 11A). In parallelized embodiments, neighboring channels share magnet assemblies. A representative COMSOL magnetic field simulation shows how magnetic flux concentrators can elevate magnetic flux gradients in regions removed from the magnets (FIG. 11B).

FIG. 12. A representative example of simulation data that guides the design of channel geometry, operational flow rates, and target magnetic field strength. Simulations approximate captured and non-captured cell trajectories passing through the serpentine gutter channels. This example results assumes a flow rate of 2 mL/hr for 0.40 mm spacing between the magnetic flux concentrator and the channel wall.

FIG. 13. Representative schematic of an embodiment of a parallelized MSD with 6 separate channels that share magnetic flux fields. In this embodiment MFCs are sandwiched by magnets with opposing polarities using fixtures in which the magnets and channels are encased. One embodiment of an MFC design is represented here where circular tabs of varying distance from the magnets concentrate the magnetic field in the vicinity of the fluidic channels.

FIG. 14. Top view of a schematic representation of one MSD embodiment in which the fluidic path wraps around a magnetic assembly comprised of magnets and a magnetic flux concentrator to extend the path length of magnetically tagged cells subject to a magnetic field.

### DETAILED DESCRIPTION OF EMBODIMENTS

Devices, methods, and kits are provided for isolating a target cell from a fluid sample. In particular, a magnetophoretic separation device is provided that applies varying magnetic field strength to flowing magnetically tagged cells. Immunomagnetic negative selection of target cells is used to maintain target cells in their native, unlabeled state. The magnetophoretic separation device is suitable for isolating cells from low volumes of whole blood, which provides an advantage over in-bulk methods that require larger starting volumes of blood. A computer implemented method is also provided for producing target magnetophoretic profiles along the path through which cells travel through a fluidic conduit in the device that is adaptable to a variety of form factors.

Before the present devices, methods, and kits are described, it is to be understood that this invention is not limited to particular devices, methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a target cell” includes a plurality of such target cells and reference to “the channel” includes reference to one or more channels and equivalents thereof, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The term “antibody” encompasses monoclonal antibodies, polyclonal antibodies, as well as hybrid antibodies, altered antibodies, chimeric antibodies, and humanized antibodies. The term antibody includes: hybrid (chimeric) antibody molecules (see, for example, Winter et al. (1991) Nature 349:293-299; and U.S. Pat. No. 4,816,567); bispecific antibodies, bispecific T cell engager antibodies (BITE), trispecific antibodies, and other multispecific antibodies (see, e.g., Fan et al. (2015) J. Hematol. Oncol. 8:130, Krishnamurthy et al. (2018) Pharmacol Ther. 185:122-134), F(ab′) 2 and F (ab) fragments; Fv molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (scFv) (see, e.g., Huston et al. (1988) Proc Natl Acad Sci USA 85:5879-5883); nanobodies or single-domain antibodies (sdAb) (see, e.g., Wang et al. (2016) Int J Nanomedicine 11:3287-3303, Vincke et al. (2012) Methods Mol Biol 911:15-26; dimeric and trimeric antibody fragment constructs; minibodies (see, e.g., Pack et al. (1992) Biochem 31:1579-1584; Cumber et al. (1992) J Immunology 149B: 120-126); humanized antibody molecules (see, e.g., Riechmann et al. (1988) Nature 332:323-327; Verhoeyan et al. (1988) Science 239:1534-1536; and U.K. Patent Publication No. GB 2,276, 169, published 21 Sep. 1994); and, any functional fragments obtained from such molecules, wherein such fragments retain specific-binding properties of the parent antibody molecule.

The terms “specific binding,” “specifically binds,” and the like, refer to non-covalent or covalent preferential binding to a molecule relative to other molecules or moieties in a solution or reaction mixture (e.g., an antibody specifically binds to a particular polypeptide (e.g., cell surface marker) or epitope relative to other available polypeptides). In some embodiments, the affinity of one molecule for another molecule to which it specifically binds is characterized by a K_{D} (dissociation constant) of 10⁻⁵ M or less (e.g., 10⁻⁶ M or less, 10⁻⁷ M or less, 108 M or less, 109 M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). “Affinity” refers to the strength of binding, increased binding affinity being correlated with a lower K_{D}.

A “ligand” or “binding agent” is any molecule that specifically binds to a cellular marker or other target. In certain embodiments, the ligand or binding agent is a molecule that selectively binds to a target analyte of interest (e.g., cellular marker) with high binding affinity. By high binding affinity is meant a binding affinity of at least about 10⁻⁴ M, usually at least about 10⁻⁶ M or higher, e.g., 10⁻⁹ M or higher. The binding agent may be any of a variety of different types of molecules, as long as it exhibits the requisite binding affinity for the target analyte when conjugated to a magnetic nanoparticle. In certain embodiments, the binding agent has medium or even low affinity for its target analyte, e.g., less than about 10⁻⁴ M. As such, the binding agent or ligand may be a small molecule or large molecule. By small molecule is meant a molecule having a size of less than 10,000 daltons, usually ranging in size from about 50 to about 5,000 daltons, and more usually from about 100 to about 1000 daltons in molecular weight. By large molecule is meant a molecule having a size of more than 10,000 daltons in molecular weight.

A small molecule binding agent or ligand may be any molecule, as well as binding portion or fragment thereof, that is capable of binding with the requisite affinity to the target analyte of interest (e.g., cellular marker). Generally, the small molecule is a small organic molecule that is capable of binding to the target analyte of interest. The small molecule will include one or more functional groups necessary for structural interaction with the target analyte, e.g., groups necessary for hydrophobic, hydrophilic, electrostatic or even covalent interactions. Where the target analyte is a protein, the drug moiety will include functional groups necessary for structural interaction with proteins, such as hydrogen bonding, hydrophobic-hydrophobic interactions, electrostatic interactions, etc., and will typically include at least an amine, amide, sulfhydryl, carbonyl, hydroxyl or carboxyl group, preferably at least two of the functional chemical groups. The small molecule will also comprise a region that may be modified and/or participate in conjugation to a fluorophore, without substantially adversely affecting the small molecule's ability to bind to its target analyte.

Small molecule ligands may comprise cyclical carbon or heterocyclic structures and/or aromatic or polyaromatic structures substituted with one or more of the above functional groups. Small molecule ligands may also include organic compounds comprising alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Small molecules may include structures found among biomolecules, including peptides, carbohydrates, fatty acids, vitamins, steroids, purines, pyrimidines, derivatives, structural analogs or combinations thereof.

The small molecule may be derived from a naturally occurring or synthetic compound that may be obtained from a wide variety of sources, including libraries of synthetic or natural compounds. For example, numerous means are available for random and directed synthesis of a wide variety of organic compounds and biomolecules, including the preparation of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known small molecules may be subjected to directed or random chemical modifications, such as acylation, alkylation, esterification, amidification, etc. to produce structural analogs.

As such, the small molecule may be obtained from a library of naturally occurring or synthetic molecules, including a library of compounds produced through combinatorial means, i.e., a compound diversity combinatorial library. When obtained from such libraries, the small molecule employed will have demonstrated some desirable affinity for the protein target in a convenient binding affinity assay. Combinatorial libraries, as well as methods for the production and screening, are known in the art and described in: U.S. Pat. Nos. 5,741,713; 5,734,018; 5,731,423; 5,721,099; 5,708,153; 5,698,673; 5,688,997; 5,688,696; 5,684,711; 5,641,862; 5,639,603; 5,593,853; 5,574,656; 5,571,698; 5,565,324; 5,549,974; 5,545,568; 5,541,061; 5,525,735; 5,463,564; 5,440,016; 5,438,119; 5,223,409, the disclosures of which are herein incorporated by reference.

As pointed out, the ligand or binding agent can also be a large molecule. Of particular interest as large molecule binding agents are antibodies, as well as binding fragments and mimetics thereof. Also suitable for use as binding agents are peptoids and aptamers. The ligand or binding agent may include a domain or moiety that can be attached to a magnetic nanoparticle without substantially abolishing the binding affinity for its target analyte (e.g., cellular marker).

The term “conjugated” refers to the joining by covalent or noncovalent means of two compounds or agents (e.g., binding agent specific for a cellular marker conjugated to a magnetic nanoparticle).

The term “specific binding member” as used herein refers to a member of a specific binding pair (i.e., two molecules, usually two different molecules, where one of the molecules, e.g., a first specific binding member, through non-covalent means specifically binds to the other molecule, e.g., a second specific binding member). Examples of binding pairs include antibody-antigen, IgE-allergen, and protein A-antibody binding pairs, and the like. Suitable specific binding members include agents that specifically bind to surface markers on activated basophils (e.g., an anti-CD63 antibody, an anti-CD203 antibody, or avidin), basophil capture agents that bind to surface markers on basophils (e.g., an anti-CD123 antibody, an anti-CD193 antibody, an anti-CD294 antibody, an anti-CCR3 antibody, an anti-CD192 antibody, and an anti-IgE antibody), and non-basophil capture agents that bind to surface markers on blood cells other than basophils (e.g., an anti-HLA-DR antibody, an anti-CD2 antibody, an anti-CD3 antibody, an anti-CD14 antibody, an anti-CD15 antibody, an anti-CD16 antibody, an anti-CD19 antibody, an anti-CD20 antibody, an anti-CD24 antibody, an anti-CD34 antibody, an anti-CD36 antibody, an anti-CD45RA antibody, an anti-CD56 antibody, an anti-CD66b antibody, and an anti-glycophorin A antibody).

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′) 2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules (2) single chain polypeptides containing only one light chain variable domain, or a fragment thereof that contains the three CDRs of the light chain variable domain, without an associated heavy chain moiety (3) single chain polypeptides containing only one heavy chain variable region, or a fragment thereof containing the three CDRs of the heavy chain variable region, without an associated light chain moiety and (4) nanobodies comprising single Ig domains from non-human species or other specific single-domain binding modules; and multispecific or multivalent structures formed from antibody fragments. In an antibody fragment comprising one or more heavy chains, the heavy chain(s) can contain any constant domain sequence (e.g. CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s).

As used in this invention, the term “epitope” means any antigenic determinant on an antigen to which the paratope of an antibody binds. Epitopic determinants usually consist of chemically active surface groupings of molecules such as amino acids or sugar side chains and usually have specific three-dimensional structural characteristics, as well as specific charge characteristics.

Obtaining and assaying a sample. The term “assaying” is used herein to include the physical steps of manipulating a biological sample to generate data related to the sample. As will be readily understood by one of ordinary skill in the art, a biological sample (e.g., blood sample containing basophils) must be “obtained” prior to assaying the sample (e.g., activation of basophils by an allergen). Thus, the term “assaying” implies that the sample has been obtained. The terms “obtained” or “obtaining” as used herein encompass the act of receiving an extracted or isolated biological sample. For example, a testing facility can “obtain” a biological sample in the mail (or via delivery, etc.) prior to assaying the sample. In some such cases, the biological sample was “extracted” or “isolated” from an individual by another party prior to mailing (i.e., delivery, transfer, etc.), and then “obtained” by the testing facility upon arrival of the sample. Thus, a testing facility can obtain the sample and then assay the sample, thereby producing data related to the sample.

The terms “obtained” or “obtaining” as used herein can also include the physical extraction or isolation of a biological sample from a subject. Accordingly, a biological sample can be isolated from a subject (and thus “obtained”) by the same person or same entity that subsequently assays the sample. When a biological sample is “extracted” or “isolated” from a first party or entity and then transferred (e.g., delivered, mailed, etc.) to a second party, the sample was “obtained” by the first party (and also “isolated” by the first party), and then subsequently “obtained” (but not “isolated”) by the second party. Accordingly, in some embodiments, the step of obtaining does not comprise the step of isolating a biological sample.

In some embodiments, the step of obtaining comprises the step of isolating a biological sample (e.g., a pre-treatment biological sample, a post-treatment biological sample, etc.). Methods and protocols for isolating various biological samples (e.g., a blood sample, a serum sample, a plasma sample, a biopsy sample, an aspirate, etc.) will be known to one of ordinary skill in the art and any convenient method may be used to isolate a biological sample.

The terms “determining”, “measuring”, “evaluating”, “assessing,” “assaying,” and “analyzing” are used interchangeably herein to refer to any form of measurement, and include determining if an element is present or not. These terms include both quantitative and/or qualitative determinations. Assaying may be relative or absolute. For example, “assaying” can be determining whether the basophil activation level is less than or “greater than or equal to” a particular threshold, (the threshold can be pre-determined or can be determined by assaying a control sample). On the other hand, “assaying to determine the basophil activation level” can mean determining a quantitative value (using any convenient metric) that represents the level of basophil activation. An individual is said to be allergic to a given allergen if the number of basophils activated by stimulation with the allergen is greater than the number of basophils activated by a negative control (i.e., control basophil activation test in absence of allergen or in presence of a non-allergenic control substance).

“Providing an analysis” is used herein to refer to the delivery of an oral or written analysis (i.e., a document, a report, etc.). A written analysis can be a printed or electronic document. A suitable analysis (e.g., an oral or written report) provides any or all of the following information: identifying information of the subject (name, age, etc.), a description of what type of biological sample(s) was used and/or how it was used, the technique used to assay the sample, the results of the assay, the assessment as to whether the individual is determined to be susceptible to an allergic reaction to an allergen, a recommendation to continue or alter therapy, a recommended strategy for additional therapy, etc. The report can be in any format including, but not limited to printed information on a suitable medium or substrate (e.g., paper); or electronic format. If in electronic format, the report can be in any computer readable medium, e.g., diskette, compact disk (CD), flash drive, and the like, on which the information has been recorded. In addition, the report may be present as a website address which may be used via the internet to access the information at a remote site.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with an allergy, allergic inflammation, anaphylaxis, etc.) as well as those in which prevention is desired (e.g., those suspected of having an allergy, etc.).

A therapeutic treatment is one in which the subject is inflicted prior to administration and a prophylactic treatment is one in which the subject is not inflicted prior to administration. In some embodiments, the subject has an increased likelihood of becoming inflicted or is suspected of being inflicted prior to treatment. In some embodiments, the subject is suspected of having an increased likelihood of becoming inflicted.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. Preferably, the mammal is human.

The term “allergy” is meant to encompass any allergic reaction resulting in activation of basophils or allergen-induced inflammation, such as caused by any ingested or inhaled allergen, occupational allergen, environmental allergen, drug, or any other substance that triggers a harmful immune reaction resulting in activation of basophils.

### A Magnetophoretic Separation Device for Isolating a Target Cell

Devices, methods, and kits are provided for isolating target cells from a fluid sample. Microfluidics has emerged as a promising technology for the precise separation and isolation of cells. Negative selection magnetic-activated cell sorting (MACS) is a popular, high-fidelity means for isolating target cell populations using antibodies and magnetic particles to remove non-target cells in a magnetic field. Current methods lack a streamlined approach for applying magnetophoretic forces that are compatible with downstream microfluidic process flows. Disclosed herein is a magnetophoretic separation device (MSD), that applies varying magnetic field strength to flowing magnetically tagged cells. Immunomagnetic negative selection of target cells is used to maintain target cells in their native, unlabeled state.

In certain embodiments, the MSD comprises: a) a single magnet or a magnetic array comprising a plurality of magnets; b) a scaffold comprising a slot to secure the single magnet or magnetic array and one or more channels, wherein each channel comprises one or more fluidic conduits connected to one or more outlets; and c) one or more magnetic flux concentrators in proximity to the one or more fluidic conduits, wherein spacing of the one or more magnetic flux concentrators relative to the single magnet or magnetic array and the one or more fluidic conduits is selected to produce a target magnetophoretic gradient profile, wherein magnetophoretic force varies along the length of the one or more fluidic conduits.

The single magnet or magnets in a magnetic array may be permanent magnets or electromagnets. In some embodiments, the magnets are rare earth magnets including, without limitation, neodymium magnets and samarium cobalt magnets. In certain embodiments, the MSD comprises a magnetic array comprising multiple magnets with opposing polarities, multiple magnets with aligned polarities, a grid of magnets with alternating polarities, or self-assembling magnetic colloids. A magnetic array may be constructed with magnets of various shapes and sizes including, without limitation, bar magnets, ring magnets, cylindrical magnets, disc magnets, rod magnets, wedge magnets, arc magnets, spherical magnets, cube magnets, block magnets, or magnets having various polygonal or other shapes. Suitable magnets are commercially available, for example, from Super Magnet Man (Pelham, AL), Amazing Magnets (Round Rock, TX), Stanford Magnets (Lake Forest, CA), K & J Magnetics (Pipersville, PA), and Great Magtech (Xiamen, China).

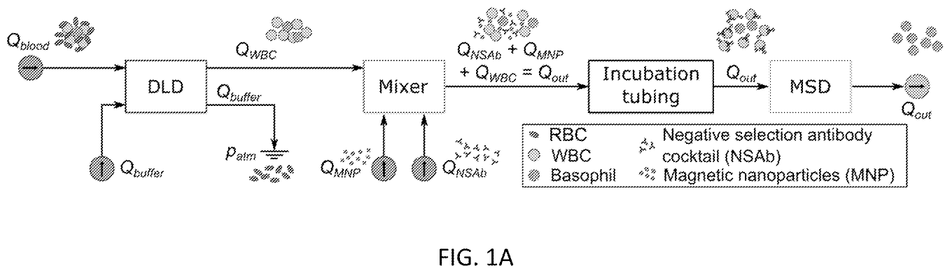

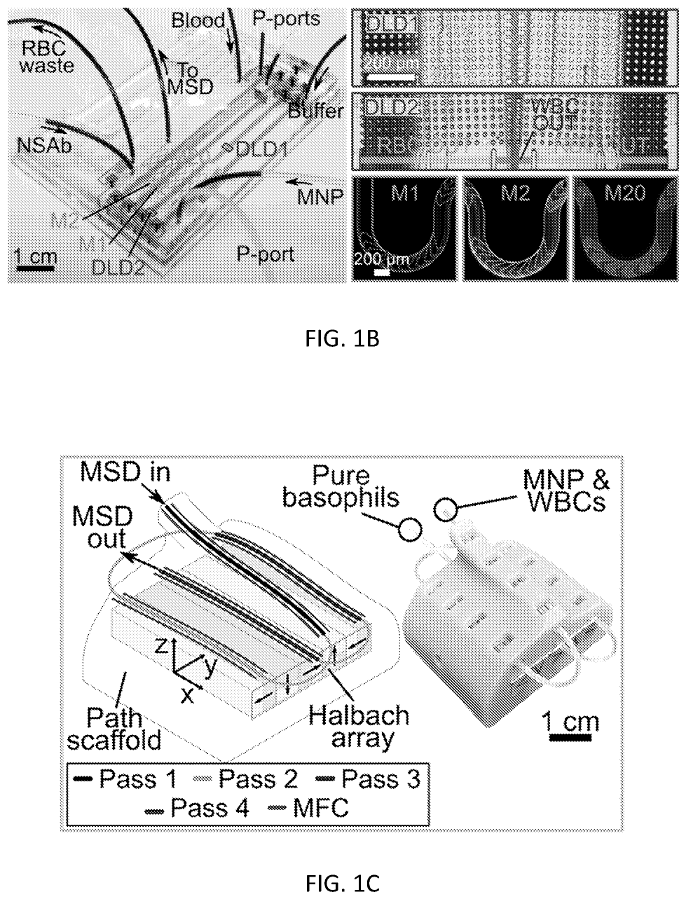

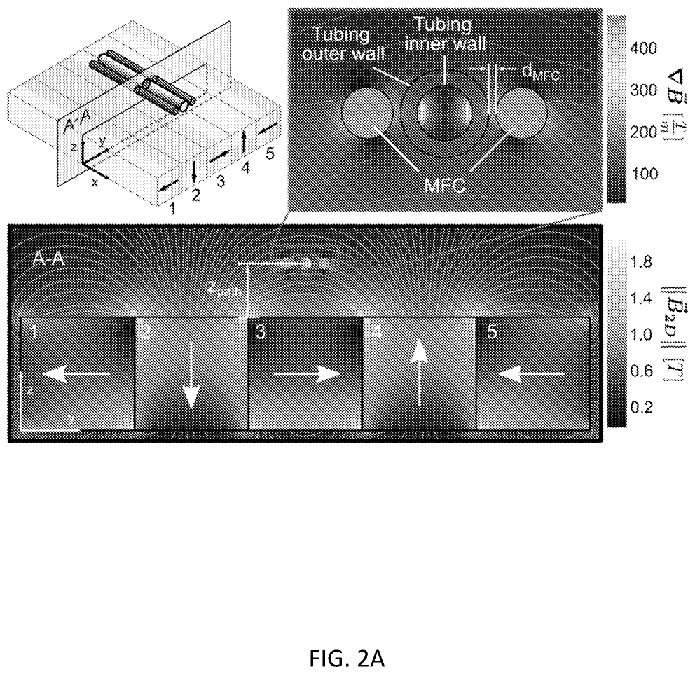

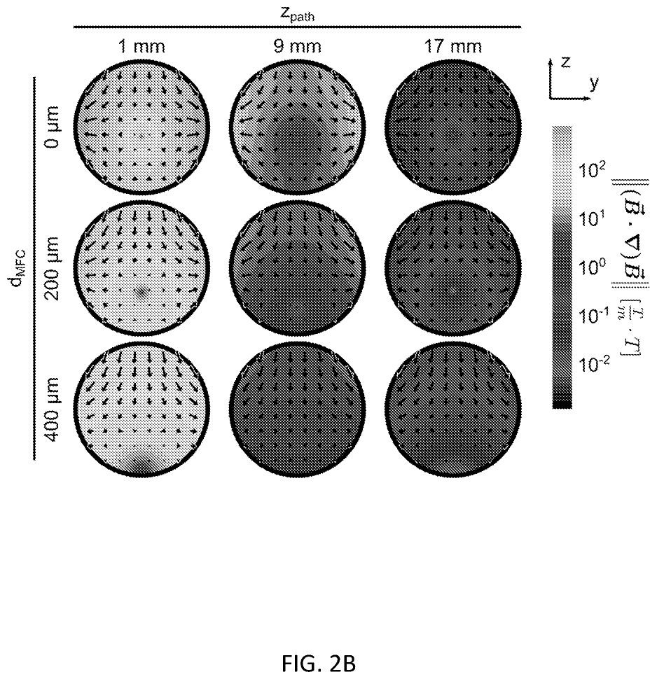

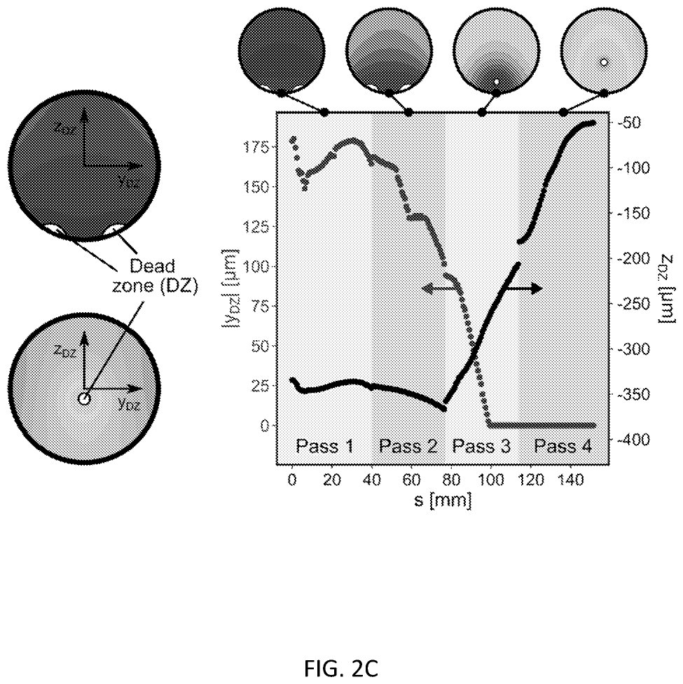

In certain embodiments, the magnetic array is a linear Halbach array. An exemplary magnetophoretic separation device with a linear Halbach array is shown in FIG. 1C. The bar magnets are arranged in a rotating pattern of magnetic orientations (e.g., bar magnets pointing left, down, right, up, left), as shown in FIG. 2A, such that the magnetic field is focused on one side of the array and near zero on the other side of the array. This rotating pattern of bar magnets can be continued with additional bar magnets to produce one-sided magnet flux. Increasing the number of magnets increases the strength of the one-sided magnetic field. In some embodiments, the MSD comprises a linear Halbach array comprising at least 5 bar magnets, at least 10 bar magnets, at least 15 bar magnets, or more. In some embodiments, the Halbach array comprises 5 bar magnets.

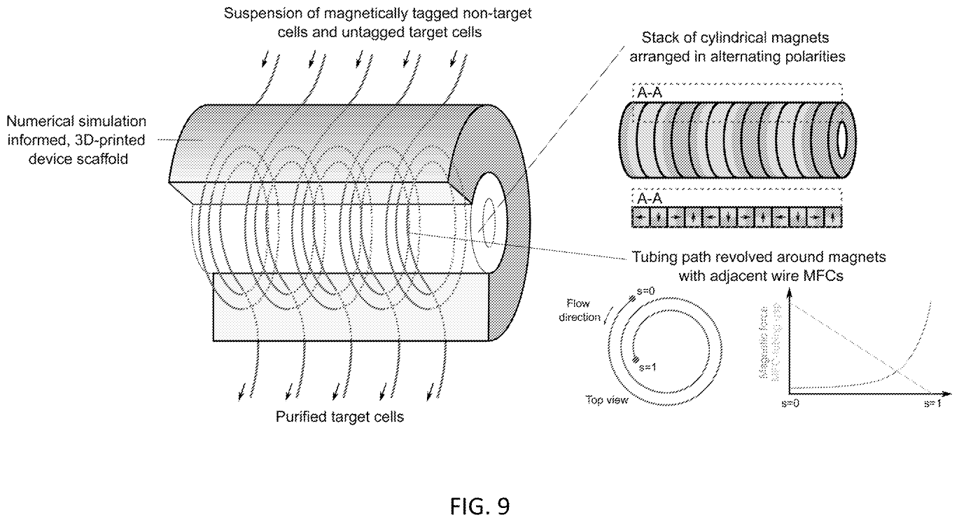

In other embodiments, the magnetic array is a cylindrical Halbach array. An exemplary magnetophoretic separation device with a cylindrical Halbach array is shown in FIG. 9. The cylindrical Halbach array comprises a stack of ring magnets arranged with alternating polarities. The orientations of the ring magnets can be designed to concentrate the magnetic field on the inner diameter of the cylinder formed from the stacked ring magnets. In some embodiments, the cylindrical Halbach array comprises at least 5 cylindrical magnets, at least 10 cylindrical magnets, at least 15 cylindrical magnets, or more. In some embodiments, the Halbach array comprises 12 cylindrical magnets.

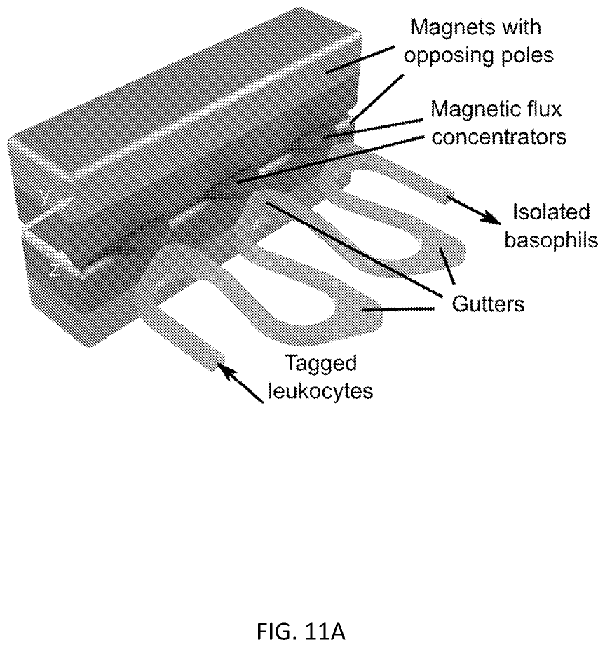

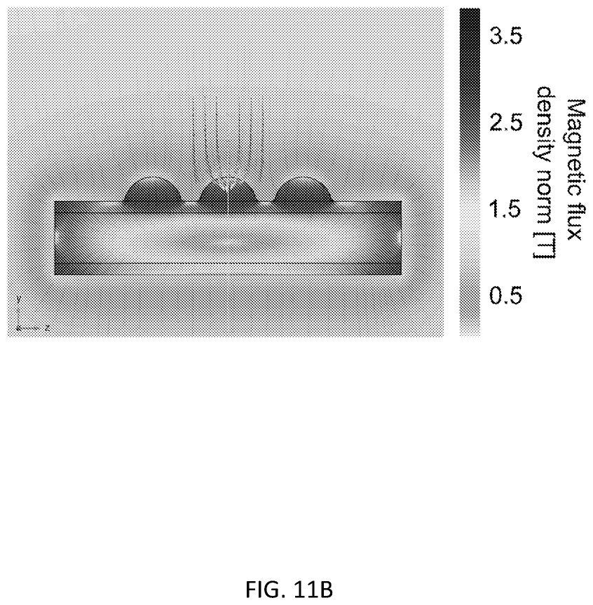

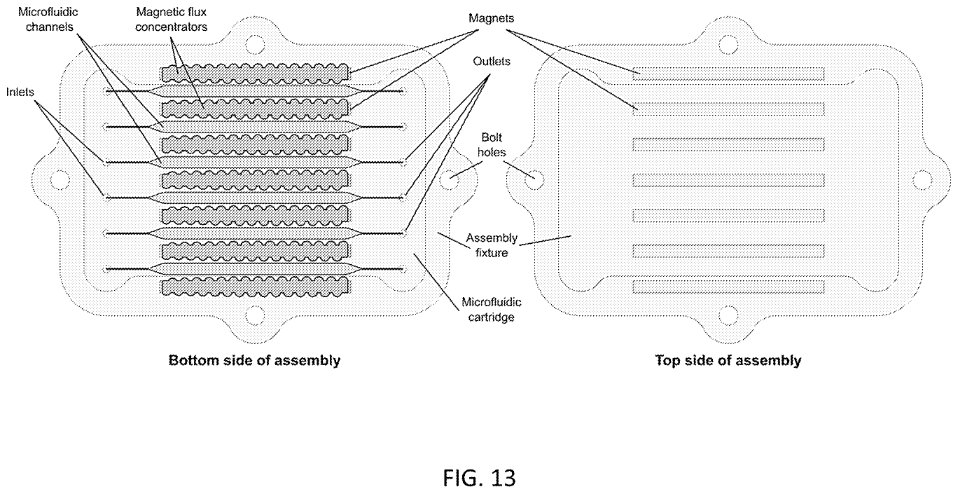

The MSD may have a single channel or multiple channels. In certain embodiments, the channel passes through the MSD in one or more passes. The geometry of channels in the MSD is designed to position the fluid flow and cell path in the vicinity of the magnetic force field and provide space for captured magnetically tagged non-target cells to accumulate. The number of captured cells in a channel can be adjusted by changing the position of the channel relative to the magnets and flux concentrator, or by changing the fluid velocity in the channel. The fluid velocity in the MSD can be adjusted by changing the input flow rate, by changing the cross-sectional area of the channel, or by a diversion of some fluid to waste. Channel geometry may be planar (e.g., embodiment represented in FIGS. 11 and 13), multi-layered, or 3-dimensional (e.g., embodiment represented in FIG. 9).

In certain embodiments, the channel width ranges from 0.01 mm to 100 mm, including any channel width within this range such as 0.01 mm, 0.05 mm, 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, or 100 mm.

In certain embodiments, the channel height ranges from 0.01 mm to 100 mm, including any channel height within this range such as 0.01 mm, 0.05 mm, 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, or 100 mm.

In certain embodiments, the channel length ranges from 1 mm to 1000 mm, including any channel height within this range such as 1 mm, 5 mm, 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, 100 mm, 120 mm, 140 mm, 160 mm, 180 mm, 200 mm, 220 mm, 240 mm, 260 mm, 280 mm, 300 mm, 320 mm, 340 mm, 360 mm, 380 mm, 400 mm, 420 mm, 440 mm, 460 mm, 480 mm, 500 mm, 520 mm, 540 mm, 560 mm, 580 mm, 600 mm, 620 mm, 640 mm, 660 mm, 680 mm, 700 mm, 720 mm, 740 mm, 760 mm, 780 mm, 800 mm, 820 mm, 840 mm, 860 mm, 880 mm, 900 mm, 920 mm, 940 mm, 960 mm, 980 mm, or 1000 mm.

In certain embodiments, the MSD comprises multiple channels, each channel comprising a separate fluidic conduit connected to a different outlet, wherein each channel can be used to separate target cells from non-target cells from a different fluid sample in parallel. Each channel outlet may be connected to a separate collection reservoir or a common collection reservoir. Each channel or a subset of channels may share the same magnetic array and magnetic flux concentrator, or each channel may have a different magnetic array and magnetic flux concentrator.

A schematic representation of an exemplary MSD comprising multiple parallel channels is shown in FIG. 13. In some embodiments, the parallel channels can be pumped using a shared pumping system using common pressure nodes across parallel channels or using a positive displacement pump at the inlet or outlet. In other embodiments, separate pumping systems are used for each individual channel. Flow rate and pressure sensors for control feedback can be applied to each parallel channel or to select channels to measure representative flow rates and pressures that are expected in all parallel channels. The outlets of the parallel channels may remain separate or outlets may flow into a common collection reservoir. In some embodiments, the flow rates in the parallel channels are identical. In other embodiments, the flow rates in the parallel channels differ. In some embodiments, parallel channels share magnet arrays and magnetic flux concentrators with other channels. In other embodiments, each parallel channel has its own magnet array and/or magnetic flux concentrator. In certain embodiments, the MSD is contained in a microfluidic cartridge comprising a first assembly comprising one or more channels and one or more magnetic flux concentrators; and a second assembly comprising the magnetic array (FIG. 13).

In certain embodiments, the fluidic conduit is provided by fluidic tubing positioned adjacent to the magnetic array or magnetic flux concentrator. The fluidic tubing preferably has an inner diameter sufficiently small that magnetic flux from the magnetic flux concentrator reaches the center axis of the fluidic tubing and sufficiently large to retain captured non-target cells without obstructing target cells from flowing through the fluidic tubing. In other embodiments, the fluidic conduit or channel is machined, embossed, rastered, or etched into a material. The fluidic conduit may provide a straight fluid path, a curved fluid path, or a serpentine fluid path. In some embodiments, a subset of channels have fluidic conduits with straight fluid paths (e.g., embodiment represented in FIG. 13), or curved fluid paths with repeating serpentine elements (e.g., embodiment represented in FIG. 11). In some embodiments, the fluid path revolves around a plurality of ring magnets in a Halbach array or runs parallel to a plurality of bar magnets in a Halbach array. In some embodiments, the fluidic conduit provides a bifurcated fluid path to direct fluid flow to regions of concentrated magnetic flux. In some embodiments, the fluid path may make multiple passes adjacent to the same magnet, magnet array or magnetic flux concentrator. The multiple passes can be in the form of the fluid path wrapping repeatedly around a magnet array or magnetic flux concentrator. The fluidic path may comprise serpentine channels, straight channels, or any other geometric configuration running adjacent to the magnet array or magnetic flux concentrator. In some embodiment, the cross-sectional shape of a fluidic conduit is square, rectangular, circular, elliptical, or polygonal.

Fluidic elements in the channels may focus all or a subset of cells towards an area of higher magnetic force field. Such fluidic elements may comprise constrictions, expansions, vanes or sheath flow devices. Focusing the flow of cells may improve the outlet purity or recovery percentage of a target cell type exiting the magnetic separation device. In some embodiments, fluidic elements may be used to mix the fluid either before reaching the MSD, or periodically within the MSD. One function of this mixing is to rearrange the cell streamlines in the channel to ensure all magnetically tagged cells experience a sufficiently large magnetic force field to be captured. This mixing may be achieved in a variety of ways, for example, by including serpentine channels, staggered herringbones, dean vortices, parallel lamination of fluid streamlines, or other microstructures such as micro-pillars or steps.

In certain embodiments, the MSD further comprises one or more gutters where captured magnetically tagged non-target cells and magnetic nanoparticles can accumulate. The use of gutters helps prevent clogging of the fluid path by preventing accumulation and filling of the full fluidic cross section of the channel with non-target cells and magnetic nanoparticles and blocking of the flow path of target cells. In some embodiments, serpentine channels are combined with gutters to place the intended accumulation area close to the region of the highest magnetic force (e.g., embodiment shown in FIG. 11). In some embodiments, the fluidic conduit bifurcates fluid flow in regions of concentrated magnetic flux to direct cells towards high magnetic field gradients.

The MSD fluidic channels with fluidic conduits and fluidic elements may be fabricated by any suitable method known in the art. Exemplary methods include lithography, additive manufacturing, casting, or forging.

In certain embodiments, the MSD comprises at least one magnetic flux concentrator, which serves to redirect and concentrate the magnetic field sufficiently close to fluidic channels to allow magnetically tagged non-target cells to be captured. Magnetic flux concentrators can be fabricated from various magnetic materials with relative magnetic permeability ranging from 100-100,000 with a magnetic saturation point ranging from 0.1-3.0 T. For example, a magnetic flux concentrator can be fabricated from a metal alloy comprising iron, nickel, and cobalt. The metal alloy material can be in the form of sheets (e.g., 10-20,000 μm), wires, extrusions, computer numerical control machining (CNC) machined parts, or metal three dimensional (3-D) printed parts. A magnetic flux concentrator can be shaped to direct magnetic flux density into a channel. The radii of magnetic flux concentrators can be adjusted to change the magnitude of grad (B). This can be used to tune the peak magnitude of the magnetic force on cells in the MSD channels, as well as the decay of the magnetic force field away from the magnetic flux concentrator. In some embodiments, an MFC comprises fluidic channels filled with ferrofluids or self-assembled magnetic colloids.

In certain embodiments, the MSD comprises a magnetic array and magnetic flux concentrator capable of producing a magnetic flux density ranging from 0.1 tesla (T) to 10 T, or at least about 0.1 T, or at least about 1 T, or at least about 2 T, or at least about 4 T, or at least about 5 T, or at least about 8 T, including any magnetic flux density within these ranges such as 0.1 T, 0.15 T, 0.2 T, 0.25 T, 0.3 T, 0.35 T, 0.4 T, 0.45 T, 0.5 T, 0.55 T, 0.6 T, 0.65 T, 0.7 T, 0.75 T. 0.8 T, 0.85 T. 0.9 T, 0.95 T, or 10 T. In certain embodiments, the magnetic flux concentrator has a relative magnetic permeability ranging from 100 to 100,000, or at least about 100, or at least about 1000, or at least about 10,000, or at least about 50,000, including any relative magnetic permeability in this range such as 100, 200, 300, 400, 500, 600, 700, 800, 900, 1000, 1500, 2000, 2500, 3000, 3500, 4000, 4500, 5000, 5500, 6000, 6500, 7000, 8000, 8500, 9000, 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, or 100,000.

In certain embodiments, a magnetic flux concentrator comprises a plurality of ferromagnetic wires, sheets, self-assembling colloids, or other configuration of ferromagnetic material running alongside a fluidic conduit, wherein spacing of the wires, sheets, self-assembling colloids, or other configuration of ferromagnetic material relative to the magnetic array and the fluidic conduit is selected to produce a desired target magnetophoretic gradient profile. In certain embodiments, the magnetic flux concentrator comprises a fluidic channel comprising a ferrofluid or a self-assembled magnetic colloid. In certain embodiments, the magnetic flux concentrator radius can be adjusted to tune peak magnitude of the magnetic force on cells in the fluidic conduit. In certain embodiments, the magnetic array in combination with the magnetic flux concentrator generate a magnetophoretic force that increases exponentially along the length of the fluidic conduit.

In certain embodiments, the MSD further comprises a plurality of pumps. In some embodiments, the pumps are syringe pumps, diaphragm pumps, peristaltic pumps or piston pumps, or any combination thereof. In some embodiments, the pumps are positive displacement pumps or pressure generating pumps. In certain embodiments, the MSD further comprises a plurality of sensors that monitor flow rate or pressure, or a combination thereof. In some embodiments, the pumps and sensors are configured to maintain the flow rate through the magnetic separation device in a range from 0.01 mL/hr to 20,000 L/hr. In certain embodiments, the one or more fluidic conduits in the MSD share magnetic arrays, magnetic flux concentrators, pumps, mixing elements, or flow through cytometers, or any combination thereof.

The magnetophoretic separation device may be included in a microfluidic device. In certain embodiments, the microfluidic magnetic device comprises: a) one or more inlets to receive the fluid sample; b) one or more mixers connected to the one or more inlets, wherein the one or more mixers mix magnetic nanoparticles (MNPs) and one or more negative selection binding agents with the cells, wherein the one or more negative selection binding agents selectively bind to one or more surface markers on non-target cells, wherein the one or more surface markers are not present on the one or more types of target cells; c) one or more incubation tubings connected to the one or more mixers, wherein the cells are incubated with the MNPs and the one or more negative selection binding agents in the one or more incubation tubings, wherein the non-target cells that are bound to the one or more negative selection binding agents are selectively magnetically labeled with the MNPs; d) an MSD described herein, wherein the one or more channels of the MSD are connected to the one or more incubation tubings, wherein the MSD captures the magnetically labeled non-target cells and outputs the one or more types of target cells; and e) one or more outlets connected to the MSD, wherein the one or more types of target cells are expelled from the one or more outlets.

In certain embodiments, the microfluidic magnetic device further comprises at least one deterministic lateral displacement (DLD) channel or a curved microchannel that sorts cells based on size, wherein the DLD channel or the curved microchannel is positioned between an inlet and a mixer, wherein an input end of the DLD channel or the curved microchannel is connected to the inlet and an output end of the DLD channel or the microchannel is connected to the mixer. In some embodiments, one or more DLD channels and mixers are contained on a microfluidic chip comprising a first layer comprising the mixers and a second layer comprising the DLD channels. In some embodiments, the first layer comprising the mixers is above the second layer comprising the DLD channels.

In some embodiments, a DLD channel further comprises a coating to reduce cell adhesion to the DLD channel. For example, the coating may include, without limitation, a non-ionic surfactant such as PLURONIC® F68 (Poloxamer 188), PLURONIC® F108 (poly(ethylene glycol)-block-poly(propylene glycol)-block-poly(ethylene glycol) diacrylate), or PLURONIC® F127 (Poloxamer 407), polyethylene glycol, poly(2-hydroxyethyl methacrylate), poly(2-methacryloyloxyethyl phosphorylcholine), PREVELEX® CC1 anti-cell adhesion coating, bovine serum albumin, or silanes such as phenyltrichlorosilane or dimethyldichlorosilane.

In certain embodiments, the microfluidic magnetic device further comprises a means for applying a secondary Dean flow velocity field or a hydrophoretic force to a curved microchannel when fluid passes through the curved microchannel. In some embodiments, the curved microchannel varies in width along the length of the curved microchannel. In some embodiments, the curved microchannel further comprises perpendicular or angled steps.

In certain embodiments, a mixer comprises a Herringbone-grooved serpentine channel. In some embodiments, the Herringbone-grooved serpentine channel has dimensions of 200 μm in width and 70 μm in height. In some embodiments, the Herringbone-grooved serpentine channel comprises a groove having a height of 30 μm.

A plurality of pumps and sensors may be used to control the flow rates and pressures in the microfluidic magnetic device. A combination of positive displacement pumps or pressure generating pumps may be used. Syringe pumps, diaphragm pumps, peristaltic pumps or piston pumps, or any combination thereof, operating under control feedback from sensors in the system may be used to monitor flow rate and/or pressure. Fluid, blood, buffer, reagents, and waste can be injected or withdrawn simultaneously across single or parallelized channels (e.g., for separate cell isolations) using any combination of pumping methods. The system may comprise a processor programmed to achieve target flow rates and pressures for operation of all microfluidic processes (e.g., enrichment of white blood cells and immunomagnetic negative selection of basophils) of the isolation system in a steady-state manner. In some embodiments, flow rates through the magnetic separation device may range from 0.01 mL/hr to 20,000 L/hr.

In some embodiments, the microfluidic device further comprises a flow-through microfluidic cytometer, for counting the number of target cells. After enrichment of the target cells by negative selection, the target cells may be flowed through a microfluidic cytometer. For a review of microfluidic cytometers and their use in microfluidic devices, see, e.g., Ehrlich et al. (2011) Methods Cell Biol. 102:49-75, Piyasena et al. (2014) Lab Chip 14 (6): 1044-1059, and Gong et al. (2018) “New advances in microfluidic flow cytometry” Electrophoresis [Epub ahead of print]; herein incorporated by reference.

### Target Cells

The microfluidic magnetic device can be used for isolation of a target cell from a fluid sample comprising a heterogenous population of cells. The cells may be in a biological fluid such as, but not limited to, blood, urine, cerebrospinal fluid, lymph fluid, bronchoalveolar lavage fluid or cells from tissue, including, but not limited to, cardiovascular tissue, connective tissue, muscle tissue, nervous tissue, or epithelial tissue. Cells or tissue may be obtained by any convenient method and are preferably obtained as aseptically as possible. For example, blood samples can be collected from a subject by venipuncture, finger prick, or heel prick. Cells or tissue can be obtained by biopsy, e.g., during endoscopy, during surgery, by needle, etc. In some embodiments, the cells or tissue are from a mammalian species such as, but not limited to a human, equine, bovine, porcine, canine, feline, rodent (e.g., mice, rats, hamster), or primate subject. The subject may be of any age, e.g., a fetus, neonate, juvenile, or adult. The cells may be obtained directly from a subject. Alternatively, the cells may be derived from the culture and expansion of a cell obtained from a subject or a cell obtained from a cell line. In some embodiments, the cell is an adult cell. In other embodiments, the cell is a progenitor cell or stem cell, or a differentiated cell derived from a progenitor cell or stem cell.

The fluid sample may or may not be subject to pre-processing, such as dilution, addition of anti-coagulants, stimulants, stains or other reagents, cell lysis, pre-enrichment of cell subtypes, physical or chemical or biochemical dissociation from tissue or cell clusters into single cells, prior to the injection into the microfluidic magnetic device.

Cells may be obtained from any part of the body of a subject, including, without limitation, from the cardiovascular system, including the heart, blood, blood vessels, and lungs; digestive system, including the salivary glands, esophagus, stomach, liver, gallbladder, pancreas, intestines, colon, rectum and anus; endocrine system, including the endocrine glands such as the hypothalamus, pituitary gland, pineal body or pineal gland, thyroid, parathyroids and adrenals (adrenal glands); excretory system, including kidneys, ureters, bladder and urethra involved in fluid balance, electrolyte balance and excretion of urine; lymphatic system, including structures involved in the transfer of lymph between tissues and the blood stream, the lymph and the nodes and vessels that transport it, the immune system, including leukocytes, tonsils, adenoids, thymus and spleen; integumentary system, including skin, hair and nails of mammals, and scales of fish, reptiles, and birds, and feathers of birds; muscular system, including skeletal, smooth and cardiac muscles; nervous system, including the brain, spinal cord, nerves, and glia; reproductive system, including the sex organs, such as ovaries, fallopian tubes, uterus, vulva, vagina, testes, vas deferens, seminal vesicles, prostate and penis; respiratory system, including the organs used for breathing, the pharynx, larynx, trachea, bronchi, lungs and diaphragm; skeletal system, including bones, cartilage, ligaments and tendons.

The target cell may be of any type such as, but not limited to, an exocrine secretory epithelial cell such as a Brunner's gland cell in the duodenum, insulated goblet cell of respiratory and digestive tracts, stomach cell such as foveolar cell (mucus secretion), a chief cell (pepsinogen secretion), parietal cell (hydrochloric acid secretion), and pancreatic acinar cell; a Paneth cell of the small intestine, a type II pneumocyte of lung, a club cell of the lung; barrier cells such as a type I pneumocyte (lung), gall bladder epithelial cell, centroacinar cell (pancreas), intercalated duct cell (pancreas), and intestinal brush border cell (with microvilli); hormone-secreting cells such as an enteroendocrine cell, K cell, L cell, I cell, G cell, enterochromaffin cell, enterochromaffin-like cell, N cell, S cell, D cell, Mo cell, thyroid gland cells, thyroid epithelial cell, parafollicular cell, parathyroid gland cells, parathyroid chief cell, oxyphil cell, pancreatic islets (islets of Langerhans), alpha cell (secretes glucagon), beta cell (secretes insulin and amylin), delta cell (secretes somatostatin), epsilon cell (secretes ghrelin), pp cell (gamma cell), cells derived primarily from ectoderm such as exocrine secretory epithelial cells, salivary gland mucous cell, salivary gland serous cell, von Ebner's gland cell in tongue, mammary gland cell, lacrimal gland cell, ceruminous gland cell in ear, eccrine sweat gland dark cell, eccrine sweat gland clear cell, apocrine sweat gland cell, gland of moll cell in eyelid, sebaceous gland cell, and bowman's gland cell in nose; hormone-secreting cells such as anterior/intermediate pituitary cells, corticotropes, gonadotropes, lactotropes, melanotropes, somatotropes, thyrotropes, magnocellular neurosecretory cells, parvocellular neurosecretory cells, and chromaffin cells (adrenal gland); epithelial cells such as a keratinocyte, epidermal basal cell, melanocyte, trichocyte, medullary hair shaft cell, cortical hair shaft cell, cuticular hair shaft cell, Huxley's layer hair root sheath cell, Henle's layer hair root sheath cell, outer root sheath hair cell, surface epithelial cell of cornea, tongue, mouth, nasal cavity, distal anal canal, distal urethra, and distal vagina, basal cell (stem cell) of cornea, tongue, mouth, nasal cavity, distal anal canal, distal urethra, and distal vagina, intercalated duct cell (salivary glands), striated duct cell (salivary glands), lactiferous duct cell (mammary glands), ameloblast, oral cells such as an odontoblast and cementoblast; nervous system cells such as neurons, sensory transducer cells such as auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor cells of retina in the eye such as photoreceptor rod cells, photoreceptor blue-sensitive cone cells of eye, photoreceptor green-sensitive cone cells of eye, and photoreceptor red-sensitive cone cells of eye; proprioceptive primary sensory neurons, touch-sensitive primary sensory neurons, chemoreceptor glomus cells of carotid body cell, outer hair cells of vestibular system of ear, inner hair cells of vestibular system of ear, taste receptor cells of taste bud, autonomic neuron cells, cholinergic neurons, adrenergic neural cells, peptidergic neural cells, sense organ and peripheral neuron supporting cells, inner pillar cells of organ of Corti, outer pillar cells of organ of Corti, inner phalangeal cells of organ of Corti, outer phalangeal cells of organ of Corti, border cells of organ of Corti, Hensen's cells of organ of Corti, vestibular apparatus supporting cells, taste bud supporting cells, olfactory epithelium supporting cells, olfactory ensheathing cells, schwann cells, satellite glial cells, enteric glial cells, central nervous system neurons and glial cells, interneurons basket cells, cartwheel cells, stellate cells, golgi cells, granule cells, lugaro cells, unipolar brush cells, martinotti cells, chandelier cells, Cajal-Retzius cells, double-bouquet cells, neurogliaform cells, retina horizontal cells, amacrine cells, starburst amacrine cells, spinal interneurons, renshaw cells, principal cells, spindle neurons, fork neurons, pyramidal cells, place cells, grid cells, speed cells, head direction cells, betz cells, stellate cells, boundary cells, bushy cells, Purkinje cells, medium spiny neurons, astrocytes, oligodendrocytes, ependymal cells, tanycytes, pituicytes, lens cells, anterior lens epithelial cell, crystallin-containing lens fiber cell; metabolism and storage cells such as adipocytes: white fat cell, brown fat cell, and liver lipocyte; secretory cells such as cells of the adrenal cortex, cells of the zona glomerulosa produce mineralocorticoids, cells of the zona fasciculata produce glucocorticoids, cells of the zona reticularis produce androgens, theca interna cell of ovarian follicle secreting estrogen, corpus luteum cell of ruptured ovarian follicle secreting progesterone, granulosa lutein cells, theca lutein cells, leydig cell of testes secreting testosterone, seminal vesicle cell, prostate gland cell, bulbourethral gland cell, Bartholin's gland cell, gland of littre cell, uterus endometrium cell, juxtaglomerular cell, macula densa cell of kidney, peripolar cell of kidney, and mesangial cell of kidney; urinary system cells such as parietal epithelial cell, podocyte, proximal tubule brush border cell, loop of henle thin segment cell, kidney distal tubule cell, kidney collecting duct cell, principal cell, intercalated cell, and transitional epithelium (lining urinary bladder); reproductive system cells such as duct cell (of seminal vesicle, prostate gland, etc.), efferent ducts cell epididymal principal cell, and epididymal basal cell; circulatory system cells, endothelial cells, extracellular matrix cells, planum semilunatum epithelial cell of vestibular system of ear, organ of Corti interdental epithelial cell, loose connective tissue fibroblasts, corneal fibroblasts (corneal keratocytes) tendon fibroblasts, bone marrow reticular tissue fibroblasts, other nonepithelial fibroblasts, pericyte, hepatic stellate cell (ito cell), nucleus pulposus cell of intervertebral disc, hyaline cartilage chondrocyte, fibrocartilage chondrocyte, elastic cartilage chondrocyte, osteoblast/osteocyte, osteoprogenitor cell, hyalocyte of vitreous body of eye, stellate cell of perilymphatic space of ear, and pancreatic stellate cell; contractile cells such as skeletal muscle cells, red skeletal muscle cell (slow twitch), white skeletal muscle cell (fast twitch), intermediate skeletal muscle cell, nuclear bag cell of muscle spindle, nuclear chain cell of muscle spindle, myosatellite cell (stem cell), cardiac muscle cell (cardiomyocyte), SA node cell, Purkinje fiber cell, smooth muscle cell (various types) myoepithelial cell of iris myoepithelial cell of exocrine glands; blood and immune system cells such as an erythrocyte (red blood cell) and precursor erythroblasts megakaryocyte (platelet precursor) platelets, a monocyte, connective tissue macrophage (various types), epidermal Langerhans cell, osteoclast (in bone), dendritic cell (in lymphoid tissues), microglial cell (in central nervous system), neutrophil granulocyte and precursors (myeloblast, promyelocyte, myelocyte, metamyelocyte), an eosinophil granulocyte and precursors, basophil granulocyte and precursors, a mast cell, helper T cell, regulatory T cell, cytotoxic T cell, natural killer T cell, B cell, plasma cell, natural killer cell, and hematopoietic stem cells; germ cells such as an oogonium/oocyte, spermatid, spermatocyte, spermatogonium cell, spermatozoon, nurse cell, granulosa cell, sertoli cell, and epithelial reticular cell; and interstitial cells such as interstitial kidney cells.

In some embodiments, the target cell is an immune cell. Immune cells, include, without limitation, basophils, monocytes, T cells (e.g., helper T cells, regulatory T cells, memory T cells, cytotoxic T cells, and gamma delta T cells), B cells (e.g., plasma cells and memory B cells), natural killer (NK) cells, dendritic cells, macrophages, neutrophils, mast cells, and eosinophils. Immune cells may be isolated from a bodily fluid such as blood or lymph fluid. Alternatively, immune cells can be obtained from bone marrow, the lymphatic system, or inflamed tissue in which immune cells have infiltrated.

In some embodiments, the target cell is a stem cell or stem cell-derived cell. Stem cells include, without limitation, hematopoietic stem cells, embryonic stem cells, mesenchymal stem cells, neural stem cells, epidermal stem cells, endothelial stem cells, gastrointestinal stem cells, liver stem cells, cord blood stem cells, amniotic fluid stem cells, skeletal muscle stem cells, smooth muscle stem cells (e.g., cardiac smooth muscle stem cells), pancreatic stem cells, olfactory stem cells, induced pluripotent stem cells; and the like. The target cells may also include cells differentiated from stem cells that can be cultured in vitro and used in a therapeutic regimen, including, but are not limited to, cardiomyocytes, keratinocytes, adipocytes, neurons, osteoblasts, pancreatic islet cells, retinal cells, and the like.

In some embodiments, the target cell is a hematopoietic stem cell (HSC), which is a mesoderm-derived cell that can be isolated from bone marrow, blood, cord blood, fetal liver and yolk sac. HSCs are characterized as CD34⁺ and CD3⁻. HSCs can repopulate the erythroid, neutrophil-macrophage, megakaryocyte and lymphoid hematopoietic cell lineages in vivo. In vitro, HSCs can be induced to undergo at least some self-renewing cell divisions and can be induced to differentiate to the same lineages as is seen in vivo. HSCs can be induced to differentiate into one or more of erythroid cells, megakaryocytes, neutrophils, macrophages, and lymphoid cells.

Neural stem cells (NSCs) are capable of differentiating into neurons, and glia (including oligodendrocytes, and astrocytes). A neural stem cell is a multipotent stem cell which is capable of multiple divisions, and under specific conditions can produce daughter cells which are neural stem cells, or neural progenitor cells that can be neuroblasts or glioblasts, e.g., cells committed to become one or more types of neurons and glial cells respectively. Methods of obtaining NSCs are known in the art.

Mesenchymal stem cells (MSCs), originally derived from the embryonal mesoderm, are multipotent cells that can differentiate to form muscle, bone, cartilage, fat, marrow stroma, and tendon. In particular, MSCs can be differentiated to produce various types of cells such as, but not limited to, cardiomyocytes, chondrocytes, melanocytes, and hepatocyte-like cells. Methods of isolating MSC are known in the art, and any known method can be used to obtain MSCs. MSCs can be isolated, for example, from bone marrow, placenta, amniotic fluid, umbilical cord blood, adipose tissue, adult muscle, corneal stroma, and dental pulp of deciduous (baby) teeth.

An induced pluripotent stem (iPS) cell is a pluripotent stem cell induced from a somatic cell, e.g., a differentiated somatic cell. iPS cells are capable of self-renewal and differentiation into cell fate-committed stem cells, including neural stem cells, as well as various types of mature cells. iPS cells can be generated from somatic cells such as skin fibroblasts and keratinocytes by genetically modifying the somatic cells with one or more expression constructs encoding Oct-3/4 and Sox2. In some embodiments, somatic cells are genetically modified with one or more expression constructs comprising nucleotide sequences encoding Oct-3/4, Sox2, c-myc, and K1f4. In some embodiments, somatic cells are genetically modified with one or more expression constructs comprising nucleotide sequences encoding Oct-4, Sox2, Nanog, and LIN28. Methods of generating iPS are known in the art, and any such method can be used to generate iPS.

In some embodiments, the devices and methods described herein are used to isolate one or more types of target cells out of a culture containing a heterogeneous mixture of undifferentiated stem cells and differentiated and partially differentiated cells derived from stem cells.

In some embodiments, the target cell is a neoplasia, tumor, or cancer cell. The target cell may be derived from neoplasia, tumors, and cancers, including benign, malignant, metastatic and non-metastatic types, and including any stage (I, II, III, IV or V) or grade (G1, G2, G3, etc.) of neoplasia, tumor, or cancer, or a neoplasia, tumor, cancer or metastasis that is progressing, worsening, stabilized or in remission. For example, the target cell may include a cell type from carcinomas, such as squamous cell carcinoma, adenocarcinoma, adenosquamous carcinoma, anaplastic carcinoma, large cell carcinoma, and small cell carcinoma. The target cell may be a cancerous cell from a cancer such as, but are not limited to, head and neck cancer, skin cancer, breast cancer, ovarian cancer, melanoma, pancreatic cancer, peripheral neuroma, glioblastoma, adrenocortical carcinoma, AIDS-related lymphoma, anal cancer, bladder cancer, meningioma, glioma, astrocytoma, cervical cancer, chronic myeloproliferative disorders, colon cancer, endometrial cancer, ependymoma, esophageal cancer, Ewing's sarcoma, extracranial germ cell tumors, extrahepatic bile duct cancer, gallbladder cancer, gastric cancer, gastrointestinal carcinoid tumors, gestational trophoblastic tumors, hairy cell leukemia, Hodgkin lymphoma, non-Hodgkin lymphoma, hypopharyngeal cancer, islet cell carcinoma, Kaposi sarcoma, laryngeal cancer, leukemia, lip cancer, oral cavity cancer, liver cancer, male breast cancer, malignant mesothelioma, medulloblastoma, Merkel cell carcinoma, metastatic squamous neck cell carcinoma, multiple myeloma and other plasma cell neoplasms, mycosis fungoides and the Sezary syndrome, myelodysplastic syndromes, nasopharyngeal cancer, neuroblastoma, non-small cell lung cancer, small cell lung cancer, oropharyngeal cancer, bone cancers, including osteosarcoma and malignant fibrous histiocytoma of bone, paranasal sinus cancer, parathyroid cancer, penile cancer, pheochromocytoma, pituitary tumors, prostate cancer, rectal cancer, renal cell cancer, retinoblastoma, rhabdomyosarcoma, salivary gland cancer, small intestine cancer, soft tissue sarcoma, supratentorial primitive neuroectodermal tumors, pineoblastoma, testicular cancer, thymoma, thymic carcinoma, thyroid cancer, transitional cell cancer of the renal pelvis and ureter, urethral cancer, uterine sarcoma, vaginal cancer, vulvar cancer, and Wilm's tumor and other childhood kidney tumors.

Cells in biological fluids can be directly loaded into a microfluidic magnetic device or MSD. In some cases, a fluid sample may be diluted, for example, to avoid clogging the device. Cells, which are obtained from tissue, can be suspended in a fluid (e.g., media or phosphate buffered saline (PBS)) and dispersed for loading into a microfluidic magnetic device or MSD.

Generally, biological samples are processed immediately or as soon as possible after they are obtained to avoid deterioration of the cells in the samples. It will be understood by one of ordinary skill in the art that in some cases, it is convenient to wait until multiple samples have been obtained prior to processing the samples. Accordingly, in some cases an isolated biological sample is stored until all appropriate samples have been obtained. One of ordinary skill in the art will understand how to appropriately store a variety of different types of biological samples and any convenient method of storage may be used (e.g., refrigeration, freezing) that is appropriate for the particular biological sample.

White Blood Cell Enrichment from Blood

In some embodiments, the microfluidic magnetic device comprises a module for isolation of a target white blood cell from a blood sample. Such a microfluidic magnetic device may comprise a means for separating red blood cells from white blood cells. Separation methods that favor high recovery of white blood cells and low incidence of microchannel fouling/clogging are preferred and may be employed upstream of magnetic separation processes.

In some embodiments, the microfluidic magnetic device comprises at least one deterministic lateral displacement (DLD) channel, wherein the DLD channel separates red blood cells from white blood cells based on size. DLD inlet channels leading to the pillar array can be designed such that a gradual increase in shear stress is applied to lessen the effect of shear induced activation of platelets and white blood cells that is caused by a sudden increase in shear at the onset of the pillar array.

In another embodiment, red blood cells are separated from white blood cells using secondary Dean flow velocity fields that form when fluid passes through a curved microchannel. Due to size differences, white blood cells and red blood cells assume different lateral equilibrium positions in these secondary flow vortices, which enable separation of white blood cells from red blood cells at the channel outlet. Another method for enrichment of white blood cells from whole blood involves applying a hydrophoretic force, which can be generated from periodic pinching and expansion of the fluid flow by periodically varying microchannel width or including perpendicular or angled steps on the channel floor. The focusing of flows deflects cells differently depending on their mass and volume, which allows the cells to be sorted into specific outlets based on their size. For example, slanted channels, which alternate position on the floor and ceiling of the channel, or angled steps, which do not run the entire width of the channel, can be used in the microfluidic magnetic device.

In other embodiment, blood cell types are separated using active methods. For example, an acoustic radiation force can be applied to separate cells based on size, density and compressibility. In another example, cells may be sorted based on their equilibrium position under a non-uniform electric field. Cell types may be separated based on their differing dielectrophoretic activity in an electric field.

Alternatively, red blood cells may be separated from white blood cells before loading the white blood cells into the microfluidic magnetic device using non-microfluidic methods. For example, red blood cells can be separated from white blood cells by bulk separation techniques, such as density gradient centrifugation.

In certain embodiments, the volume of the blood sample loaded into a microfluidic device ranges from at least 5 μl, at least 10 μl, at least 25 μl, at least 50 μl, at least 100 μl, at least 250 μl, or at least 500 μl up to 1 m1, including any volume within this range, such as 5 μl, 10 μl, 20 μl, 30 μl, 40 μl, 50 μl, 60 μl, 70 μl, 80 μl, 90 μl, 100 μl, 120 μl, 140 μl, 160 μl, 180 μl, 200 μl, 225 μl, 250 μl, 300 μl, 350 μl, 400 μl, 450 μl, 500 μl, 550 μl, 600 μl, 700 μl, 750 μl, 800 μl, 850 μl, 900 μl, 950 μl, or 1000 μl of blood.

The microfluidic devices involved in the processing of blood can be designed and operated such that expected shear stresses remain below or equal to physiological shear stresses in blood vessels. This ensures negligible artificial activation of basophils due to shear stresses. In some embodiments, the maximum allowable shear stress under a given channel geometry and flow rate is 40 dyn/cm².

### Binding Agents

Immunomagnetic negative selection of a target cell is carried out with binding agents that selectively bind to surface markers on non-target cells. Examples of binding agents include, without limitation, antibodies, antibody fragments, antibody mimetics, and aptamers as well as small molecules, peptides, peptoids, or ligands that bind selectively to cellular markers. In some embodiments, multiple binding agents are used for negative selection, wherein the different binding agents bind to different markers on non-target cells of the same cell-type or different cell-types.

In certain embodiments, the binding agent comprises an antibody that specifically binds to a marker on a non-target cell. Any type of antibody may be used, including, without limitation, monoclonal antibodies, polyclonal antibodies, as well as hybrid antibodies, altered antibodies, chimeric antibodies, and humanized antibodies. Antibodies may include hybrid (chimeric) antibody molecules (see, for example, Winter et al. (1991) Nature 349:293-299; and U.S. Pat. No. 4,816,567); F(ab′) 2 and F (ab) fragments; Fv molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (scFv) (see, e.g., Huston et al. (1988) Proc Natl Acad Sci USA 85:5879-5883); nanobodies or single-domain antibodies (sdAb) (see, e.g., Wang et al. (2016) Int J Nanomedicine 11:3287-3303, Vincke et al. (2012) Methods Mol Biol 911:15-26; dimeric and trimeric antibody fragment constructs; minibodies (see, e.g., Pack et al. (1992) Biochem 31:1579-1584; Cumber et al. (1992) J Immunology 149B: 120-126); diabodies, tetrabodies, affibodies, camelid antibodies, humanized antibody molecules (see, e.g., Riechmann et al. (1988) Nature 332:323-327; Verhoeyan et al. (1988) Science 239:1534-1536; and U.K. Patent Publication No. GB 2,276, 169, published 21 Sep. 1994); and, any functional fragments obtained from such molecules, wherein such fragments retain specific-binding properties of the parent antibody molecule.

In other embodiments, the binding agent comprises an aptamer that specifically binds to a marker on a non-target cell. Any type of aptamer may be used, including a DNA, RNA, xeno-nucleic acid (XNA), or peptide aptamer that specifically binds to a marker on a non-target cell. Such aptamers can be identified, for example, by screening a combinatorial library. Nucleic acid aptamers (e.g., DNA or RNA aptamers) that bind selectively to a cellular marker can be produced by carrying out repeated rounds of in vitro selection or systematic evolution of ligands by exponential enrichment (SELEX). Peptide aptamers that bind to a marker of interest may be isolated from a combinatorial library and improved by directed mutation or repeated rounds of mutagenesis and selection. For a description of methods of producing aptamers, see, e.g., Aptamers: Tools for Nanotherapy and Molecular Imaging (R. N. Veedu ed., Pan Stanford, 2016), Nucleic Acid and Peptide Aptamers: Methods and Protocols (Methods in Molecular Biology, G. Mayer ed., Humana Press, 2009), Nucleic Acid Aptamers: Selection, Characterization, and Application (Methods in Molecular Biology, G. Mayer ed., Humana Press, 2016), Aptamers Selected by Cell-SELEX for Theranostics (W. Tan, X. Fang eds., Springer, 2015), Cox et al. (2001) Bioorg. Med. Chem. 9 (10): 2525-2531; Cox et al. (2002) Nucleic Acids Res. 30 (20): e108, Kenan et al. (1999) Methods Mol. Biol. 118:217-231; Platella et al. (2016) Biochim. Biophys. Acta November 16 pii: S0304-4165 (16) 30447-0, and Lyu et al. (2016) Theranostics 6 (9): 1440-1452; herein incorporated by reference in their entireties.

In other embodiments, the binding agent comprises an antibody mimetic that selectively binds to a marker on a non-target cell. Any type of antibody mimetic may be used, including, but not limited to, affibody molecules (Nygren (2008) FEBS J. 275 (11): 2668-2676), affilins (Ebersbach et al. (2007) J. Mol. Biol. 372 (1): 172-185), affimers (Johnson et al. (2012) Anal. Chem. 84 (15): 6553-6560), affitins (Krehenbrink et al. (2008) J. Mol. Biol. 383 (5): 1058-1068), alphabodies (Desmet et al. (2014) Nature Communications 5:5237), anticalins (Skerra (2008) FEBS J. 275 (11): 2677-2683), avimers (Silverman et al. (2005) Nat. Biotechnol. 23 (12): 1556-1561), darpins (Stumpp et al. (2008) Drug Discov. Today 13 (15-16): 695-701), fynomers (Grabulovski et al. (2007) J. Biol. Chem. 282 (5): 3196-3204), and monobodies (Koide et al. (2007) Methods Mol. Biol. 352:95-109).

In other embodiments, the binding agent comprises a small molecule ligand. Small molecule ligands encompass numerous chemical classes, e.g., small organic compounds having a molecular weight of less than about 10,000 daltons, less than about 5,000 daltons, or less than about 2,500 daltons. The small molecule will include one or more functional groups necessary for structural interaction with the target analyte, e.g., groups necessary for hydrophobic, hydrophilic, electrostatic or even covalent interactions. Where the target analyte is a protein (e.g., cellular marker), the ligand will include functional groups necessary for structural interaction with proteins, such as hydrogen bonding, hydrophobic-hydrophobic interactions, electrostatic interactions, etc., and will typically include at least an amine, amide, sulfhydryl, carbonyl, hydroxyl or carboxyl group, or preferably at least two of the functional chemical groups. The small molecule may also comprise a region that may be modified and/or participate in conjugation to a fluorophore, without substantially adversely affecting the small molecule's ability to bind to its target analyte.

Small molecule ligands can comprise cyclical carbon or heterocyclic structures and/or aromatic or polyaromatic structures substituted with one or more of the above functional groups. Small molecule ligands may also include organic compounds comprising alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Small molecule ligands are also found among biomolecules including peptides, carbohydrates, fatty acids, steroids, purines, pyrimidines, derivatives, structural analogs, or combinations thereof. The small molecule may be derived from a naturally occurring or synthetic compound that may be obtained from a wide variety of sources, including libraries of synthetic or natural compounds. For example, numerous means are available for random and directed synthesis of a wide variety of organic compounds and biomolecules, including the preparation of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known small molecules may be subjected to directed or random chemical modifications, such as acylation, alkylation, esterification, amidification, etc. to produce structural analogs.

As such, the small molecule may be obtained from a library of naturally occurring or synthetic molecules, including a library of compounds produced through combinatorial means, i.e., a compound diversity combinatorial library. When obtained from such libraries, the small molecule employed will have demonstrated some desirable affinity for the protein target in a convenient binding affinity assay. Combinatorial libraries, as well as methods for the production and screening, are known in the art and described in: U.S. Pat. Nos. 5,741,713; 5,734,018; 5,731,423; 5,721,099; 5,708,153; 5,698,673; 5,688,997; 5,688,696; 5,684,711; 5,641,862; 5,639,603; 5,593,853; 5,574,656; 5,571,698; 5,565,324; 5,549,974; 5,545,568; 5,541,061; 5,525,735; 5,463,564; 5,440,016; 5,438,119; 5,223,409, the disclosures of which are herein incorporated by reference.

Magnetic nanoparticles may be conjugated to binding agents by any suitable method. In some instances, the magnetic nanoparticle and binding agent may be directly linked, e.g., via a single bond, or indirectly linked e.g., through the use of a suitable linker, e.g., a polymer linker, a chemical linker, or one or more linking molecules or moieties. In some instances, attachment of the binding agent to the magnetic nanoparticle may be by way of one or more covalent interactions. In some instances, the magnetic nanoparticle or binding agent may be functionalized, e.g., by addition or creation of a reactive functional group. Functionalized magnetic nanoparticles or binding agents may be modified to contain any convenient reactive functional group for conjugation such as an amine functional group, a carboxylic functional group, a sulfhydryl group, a thiol functional group, and the like.

Any convenient method of bioconjugation may be used including, but not limited to, glutaraldehyde crosslinking, carbodiimide crosslinking, succinimide ester crosslinking, imidoester, crosslinking, maleimide crosslinking, iodoacetamide crosslinking, benzidine crosslinking, periodate crosslinking, isothiocyanate crosslinking, and the like. Such conjugation methods may optionally use a reactive sidechain group of an amino acid residue of the binding agent (e.g., a reactive side-chain group of a Lys, Cys, Ser, Thr, Tyr, His or Arg amino acid residue of the protein, i.e., a polypeptide linking group may be amino-reactive, thiol-reactive, hydroxyl-reactive, imidazolyl-reactive or guanidinyl-reactive). In some cases, a chemoselective reactive functional group may be utilized. Other conjugation reagents that can be used include, but are e.g., not limited to, homobifunctional conjugation reagents (e.g., (bis(2-[succinimidooxycarbonyloxy]ethyl) sulfone, 1,4-Di-(3′-[2′pyridyldithio]-propionamido) butane, disuccinimidyl suberate, disuccinimidyl tartrate, sulfodisuccinimidyl tartrate, dithiobis(succinimidyl propionate), 3,3′-dithiobis(sulfosuccinimidyl propionate), ethylene glycol bis(succinimidyl succinate), and the like), heterobifunctional conjugation reagents (e.g., m-maleimidobenzoyl-N-hydroxysuccinimide ester, m-maleimidobenzoyl-N-hydroxysulfosuccinimide ester, N-γ-maleimidobutyryloxysuccinimide ester, N-γ-maleimidobutyryloxysulfosuccinimide ester, N-(8-maleimidocaproic acid) hydrazide, N-(ε-maleimidocaproyloxy) succinimide ester, N-(8-maleimidocaproyloxy) sulfo succinimide ester, N-(p-maleimidophenyl) isocyanate, N-succinimidyl (4-iodoacetyl)aminobenzoate, succinimidyl 4-(N-maleimidomethyl)cyclohexane-I-carboxylate, succinimidyl 4-(p-maleimidophenyl) butyrate, N-sulfosuccinimidyl(4-iodoacetyl)aminobenzoate, sulfosuccinimidyl 4-(N-maleimidomethyl)cyclohexane-I-carboxylate, sulfo succinimidyl 4-(p-maleimidophenyl) butyrate, I-ethyl-3-(3-dimethylaminopropyl) carbodiimide hydrochloride, I-ethyl-3-(3-dimethylaminopropyl) carbodiimide hydrochloride, maleimide PEG N-hydroxysuccinimide ester, and the like), photoreactive conjugation reagents (e.g., p-azidobenzoyl hydrazide, N-5-azido-2-nitrobenzyloxysuccinimide, p-azidophenyl glyoxal monohydrate, N-(4-[p-azidosalicylamido]butyl)-3′-(2′-pyridyldithio) propionamide, bis(P-[4-azidosalicylamido]-ethyl)disulfide, N-hydroxysuccinimideyl-4-azidosalicyclic acid, N-hydroxysulfosuccinimidyl-4-azidobenzoate, sulfosuccinimidyl 2-(7-azido-4-methylcoumarin-3-acetamide)ethyl-1,3-dithiopropionate, azido phenyl 2-(m-azido-o-nitrobenzamido)-ethyl-1,3′-propionate, sulfosuccinimidyl 6-(4′-azido-2′-nitrophenylamino) hexanoate, sulfosuccinimidyl(4-azidophenyl dithio) propionate, sulfosuccinimidyl-2-(p-azidosalicylamido)ethyl-1,3-dithiopropionate, and the like).

In some instances, attachment of a binding agent to a magnetic nanoparticle is mediated by one or more functional linkers. A functional linker, as used herein, refers to any suitable linker that has one or more functional groups for the attachment of one molecule to another. For example, in some instances the functional linker comprises an amino functional group, a thiol functional group, a hydroxyl functional group, an imidazolyl functional group, a guanidinyl functional group, an alkyne functional group, an azide functional group, or a strained alkyne functional group. Further exemplary functional groups and methods of crosslinking and conjugation are described in, e.g., Hermanson Bioconjugate Techniques (Academic Press, 3rd edition, 2013), herein incorporated by reference in its entirety.

In some embodiments, the target cell is a basophil, which is isolated from a blood sample using one or more binding agents capable of selectively binding to one or more cell surface markers that are present on non-basophils but not on basophils in the blood sample. Such binding agents may comprise an antibody, antibody mimetic, aptamer, peptoid, or ligand specific for a cell surface marker on non-basophils. In some embodiments, the binding agent comprises at least one antibody selected from the group consisting of an anti-HLA-DR antibody, an anti-CD2 antibody, an anti-CD3 antibody, an anti-CD14 antibody, an anti-CD15 antibody, an anti-CD16 antibody, an anti-CD19 antibody, an anti-CD20 antibody, an anti-CD24 antibody, an anti-CD34 antibody, an anti-CD36 antibody, an anti-CD45RA antibody, an anti-CD56 antibody, an anti-CD66b antibody, and an anti-glycophorin A antibody.

### Measuring Basophil Activation

Basophils isolated using a microfluidic magnetic device, described herein, can be used in allergy screening. Clinically, the activation of basophils in the presence of allergens ex vivo correlates with the allergic status of a subject. Blood samples can be collected from a subject, for example, by venipuncture, finger prick, or heel prick. The concentration of basophils in whole blood is typically approximately 10⁴ to 10⁵ cells/ml, which is sufficient for isolation by a microfluidic magnetic device, as described herein, for further analysis. Generally, blood samples are processed immediately or as soon as possible after they are obtained to avoid deterioration of the samples and loss of the capacity of basophils to be activated by stimulation with allergens. In some cases, blood samples are stored at a cold temperature, for example at 4 degrees Celsius, for up to 24 hours. It will be understood by one of ordinary skill in the art that in some cases, it is convenient to wait until multiple samples (e.g., multiple blood samples each stimulated with a different test allergen) have been obtained prior to assaying the samples. Accordingly, in some cases an isolated biological sample (e.g., a blood sample stimulated with a test allergen) is stored until all appropriate samples have been obtained. One of ordinary skill in the art will understand how to appropriately store a variety of different types of biological samples and any convenient method of storage may be used (e.g., refrigeration, freezing) that is appropriate for the particular biological sample.

In certain embodiments, the basophils, activated by a test allergen, are detected by contacting them with at least one detectably labeled binding agent capable of selectively binding to a basophil activation marker. Such binding agents may comprise an antibody, aptamer, or ligand specific for a cell surface marker on the activated basophils. Exemplary basophil activation markers include CD63 and CD203. In some embodiments, the detectably labeled binding agent comprises an antibody specific for a basophil activation marker covalently linked to a detectable label. Exemplary antibodies that may be used in the binding agent to detect basophil activation include an anti-CD203 antibody (detects early basophil activation), an anti-CD63 antibody (detects late basophil activation), and an anti-avidin antibody (used in combination with avidin to detect basophil degranulation). In other embodiments, the detectably labeled binding agent comprises avidin covalently linked to a detectable label for detection of degranulation by activated basophils. Avidin stains negatively charged proteoglycans of the basophil granule matrix that become exposed upon degranulation. After binding of the binding agents to the activated basophils, any unbound binding agent may be removed before detection of the activated basophils, for example by rinsing the basophils.

The binding agents specific for cellular markers of non-target cells (e.g., antibodies, antibody mimetics, aptamers, peptoids, or ligands), which are used in enriching basophils by negative selection and/or labeling basophils activated by an allergen should be used in an amount sufficient to bind to the applicable blood cells. For example, the amount of a binding agent used will typically range from about 0.5 μg to about 50 μg, including any amount within this range, such as 0.5 μg, 1 μg, 2 μg, 3 μg, 4 μg, 5 μg, 6 μg, 7 μg, 8 μg, 9 μg, 10 μg, 15 μg, 20 μg, 25 μg, 30 μg, 35 μg, 40 μg, 45 μg, or 50 μg. The exact amount needed will vary and depend on the particular binding agent selected and its affinity for a particular cellular marker.

In certain embodiments, the microfluidic device comprises a fluorescence detector for detecting activated basophils having fluorescently labeled binding agents bound to them. The fluorescence detector may be located at a position in the microfluidic device such that the fluorescence detector is capable of detecting fluorescence emitted from the captured activated basophils within the second region. Alternatively, activated basophils, after labeling with fluorescently labeled binding agents that bind to their activation markers, may be flowed to another site (i.e., detection region) in the microfluidic site for detection by a fluorescence detector positioned, for example, downstream of the second region in the microfluidic device. For detection, the activated basophils are illuminated with an exterior light source at a fluorescence excitation wavelength of the fluorescent label on the binding agents. Excitation light sources that can be used include arc lamps and lasers, laser diodes and other light emitting diode sources, and both single and multiple photon excitation sources.

In some embodiments, basophil activation is measured using an immunoassay with an antibody specific for a basophil activation marker. Any suitable immunoassay technique known in the art may be used including, without limitation, an enzyme-linked immunosorbent assay (ELISA), a radioimmunoassay (RIA), or an immunofluorescent assay (IFA). For example, basophil activation can be measured using an ELISA with basophil activation markers (e.g., CD203c or CD63) conjugated to, e.g., horseradish peroxidase (HRP), alkaline phosphatase (AP), or other enzyme that enables the production of a detectable signal with a chromogenic substrate. Examples of ELISA enzyme-substrate combinations that can be used include without limitation, HRP with chromogenic substrates such as but not limited to, AmplexRed or QuantaRed, which produce fluorescent products (resorufin) that can be detected, o-phenylenediamine dihydrochloride (OPD), which produces an orange-brown product that can be detected spectrophotometrically, 2,2′-azinobis [3-ethylbenzothiazoline-6-sulfonic acid]-diammonium salt (ABTS) which produces a green product that can be detected spectrophotometrically; and AP with chromogenic substrates such as p-nitrophenyl phosphate (PNPP), which produces a yellow product that can be detected spectrophotometrically, and 5-bromo-4-chloro-3-indolyl phosphate (BCIP), which produces a blue/purple product that can be detected spectrophotometrically. Activation of basophils results in more intense signals for a basophil-specific marker that increases expression during activation. As CD203c is present on non-activated basophils and is upregulated upon activation, the CD203c marker provides a means for a single-marker readout for detecting basophils, both activated and non-activated, within a compartment of white blood cells.

In some embodiments, a coupled enzyme reaction may be used to improve specificity in detecting activated basophils. For example, histamine, a basophil-derived mediator indicative of activation, can be detected using an enzymatic reaction catalyzed by diamine oxidase, which produces H₂O₂ as a byproduct. H₂O₂ produced by this reaction can be detected using a peroxidase with a chromogenic substrate as described above (e.g., detecting oxidation of AmplexRed to resorufin). Since histamine is a mediator released by basophils after stimulation, and CD203c surface markers are upregulated by basophils upon stimulation, a coupled reaction of the histamine assay and CD203c conjugated to peroxidase will detect cells having high expression of CD203c and histamine secretion. For example, cells that express high levels of CD203c and secrete histamine are identified as activated basophils; cells that are low in histamine or low in CD203c are identified as non-basophils or non-activated basophils; cells that are high in histamine and low in CD203c are identified as non-basophils or non-activated basophils; and cells that are low in histamine and high in CD203c are identified as non-basophils or non-activated basophils. This increases specificity by filtering out cells that might have upregulated CD203c which are not activated basophils, while also eliminating cells that secrete histamine and are not activated basophils.

In some embodiments, an optical method is used to detect basophil activation that utilizes silver reduction chemistry. For example, silver reduction chemistry can be used in combination with an ELISA-based method with a secondary antibody that is conjugated to gold nanoparticles. The secondary antibodies bind to primary antibodies used for labeling a specified analyte (e.g., basophil activation marker or secretory product). In a solution of silver nitrate and hydroquinone, gold colloids catalyze the reduction of silver ions to silver atoms to form a silver film, which has an optical density that correlates with the analyte concentration. In this assay, the analyte may be a cell surface marker such as CD203c, or a mediator secreted by basophils or other white blood cells, such as histamine.

In other embodiments, the microfluidic device comprises a sensor capable of detecting changes in electrical or electrochemical current associated with activation of the basophils by an allergen. For example, the sensor may comprise a microelectrode. During degranulation of basophils due to stimulation by an allergen, negatively charged proteoglycans are exposed on the surface of the cell membrane. This alters the surface charge and membrane capacitance of the cell. In some embodiments, basophil activation is measured by an electrical method, such as by measuring impedance to detect the change in surface charge and/or membrane capacitance. Basophils flowing past a neighboring electrode will cause a change in impedance measured between the electrodes. Activated basophils are identified by a different impedance change compared with non-activated basophils.

In some embodiments, basophil activation is monitored over time with a series of measurements. As some embodiments rely on enzymatic reactions, time-varying measurements of signals can be used in monitoring basophil activation. In addition to end-point readouts, i.e., after basophils have been labeled with a detectable label (fluorescent or chemiluminescent label), time dependent measurements indicate rates of enzymatic reactions and improve detection of activated basophils. Furthermore, time series measurements of basophil impedance can also provide correlations to patient-specific rates of allergic reactions.

### Kits

Also provided are kits comprising a magnetophoretic separation device or a microfluidic magnetic device for isolation of a target cell from a fluid sample, as described herein. The kit may also comprise one or more negative selection binding agents (e.g., an antibody, antibody mimetic, peptoid, or aptamer) that selectively binds to one or more surface markers on non-target cells, wherein the surface markers are not present on the target cell. In certain embodiments, the target cell is an immune cell, a blood cell, a stem cell, or a cancer cell. In some embodiments, the target cell is a basophil, a neutrophil, an eosinophil, a mast cell, a monocyte, a dendritic cell, a macrophage, a T cell, a B cell, a natural killer cell, or a stem cell. The subject kits may further comprise media, buffers, magnetic nanoparticles, fluidic tubing, and the like.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

### Systems and Computer Implemented Methods

The present disclosure provides systems and computer implemented methods which find use in practicing the subject methods. In some embodiments, the system may include a processor programmed for optimizing positioning of a fluidic conduit (e.g., fluidic tubing) and a magnetic flux concentrator (MFC) relative to the plurality of magnets of the magnetophoretic separation device (MSD). The processor may be programmed to perform steps of the computer implemented method comprising: modeling magnetic field and magnetophoretic forces at different locations relative to a magnetic array (e.g., Halbach array); and identifying a set of spatial coordinates to position a fluidic conduit (e.g., fluidic tubing) and the magnetic flux concentrator relative to the plurality of magnets to achieve a target exponential magnetophoretic force profile along the fluidic tubing. In some embodiments, the computer implemented method comprises: (a) generating a target mathematical profile of max ∥(∇·B̅)B̅∥ as a function of the path of the fluidic tubing s, wherein the upper bound of the target value for max ∥(∇·B̅)B̅∥ metric is set according to the highest value of max ∥(∇·B̅)B̅∥ for zₚₐₜₕ=1 mm, or any other appropriate zero position of the fluidic path, and d_{MFC}=0 mm, or any other appropriate zero position for the flux concentrator, and where the scalar distance to the fluid path zₚₐₜₕ is defined in any direction relative to the magnet array or flux concentrator (b) identifying different combinations of zₚₐₜₕ and d_{MFC} that could generate a target magnetophoretic force profile along the path of the tubing by plotting max ∥(∇·B̅)B̅∥ obtained from a parametric sweep on a surface as a function of zₚₐₜₕ and d_{MFC}; (c) identifying candidate paths (q) on the max ∥(∇·B̅)B̅∥ surface that would generate a target profile f(s); (d) translating the candidate parametric paths into physical path positions; (e) performing a high-resolution numerical simulation of the magnetic field in 3-dimensional (3D) free space over the magnetic array to identify a set of (x, y, z) points in the simulation that satisfy the following two criteria: 1) d\vec{B}(x,y,z)/dy≈0, and 2) ∥\vec{B}_{3D,yz}(x,y,z)∥≈∥\vec{B}_{2D}(zₚₐₜₕ)∥; (f) displaying sets of 3D candidate paths translated from parameter space that achieve the target magnetophoretic force profile; (g) evaluating 3D candidate paths using a cost function to select for a path that minimizes the following functions:

$${α}_{1} = max ( \frac{dz}{dx} ) - 1 , \qquad 1 )$$

which selects for paths with gradual slopes for the tubing to follow in the xz-plane,

$${α}_{2} = {\int }_{0}^{L} {d}_{MFC} dx - \frac{0.45 L}{2} , \qquad 2 )$$

which selects for MFC paths with gradually varying d_{MFC} from 450 to 0 μm across the total length L of the path to facilitate the 3D printing and the threading of the MFC. 3) Σⱼ₌₁^{N}|∥\vec{B}_{2D,j}∥−∥\vec{B}_{3D,yz,j}∥|, which selects for paths that can best match ∥\vec{B}_{3D,yz}(x, y, z)∥ with ∥\vec{B}_{2D}(zₚₐₜₕ)∥ across all N points in the path; and (h) selecting the path e with the minimum cost function

$${min}_{θ} \frac{1}{3} {\sum }_{i = 1}^{3} {[ {α}_{i} ( θ ) ]}^{2}$$

as the final position of the fluidic tubing and the MFC to construct the MSD.

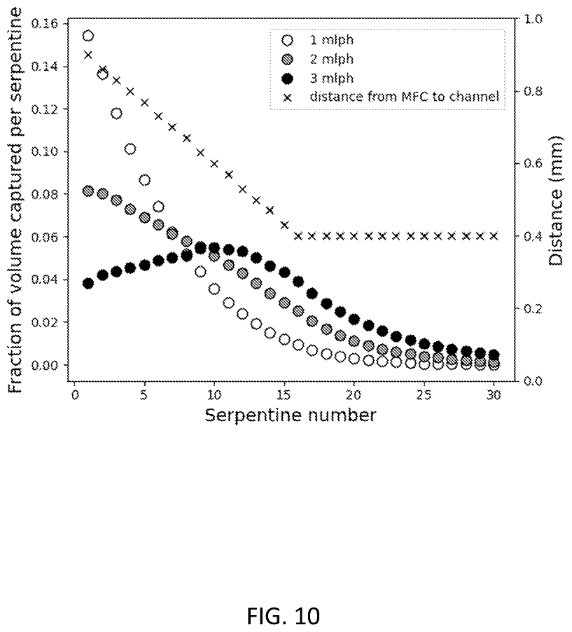

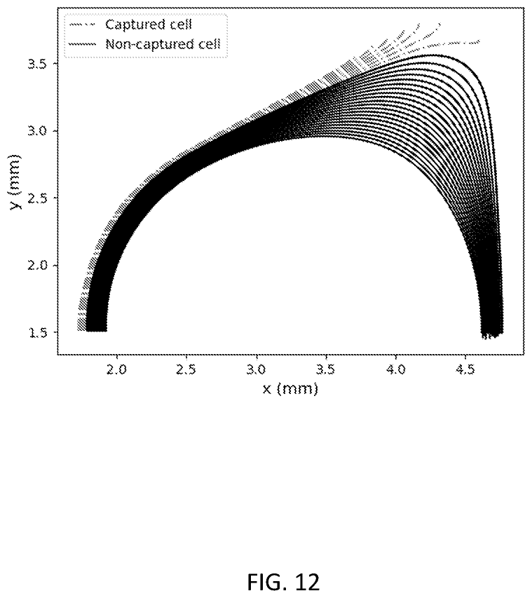

In some embodiments, the system may include: a processor programmed to determining positioning of a fluidic conduit (e.g., fluidic tubing) and a magnetic flux concentrator (MFC) relative to the plurality of magnets of a magnetophoretic separation device (MSD), wherein the computer performs steps comprising: modeling magnetic field and magnetophoretic forces at different locations relative to the magnetic array; and modeling fluidic forces on the cells and the magnetic nanoparticles present within the channel; and identifying a set of spatial coordinates to position the fluidic conduit and the magnetic flux concentrator relative to the plurality of magnets to achieve a target captured cell density along the fluidic conduit. In some embodiments, the processor may be programmed to perform steps of the computer implemented method comprising: a) generating a target mathematical cell capture profile for capture of magnetically labeled non-target cells; b) performing a high-resolution numerical simulation of the magnetic field in 3-dimensional (3D) free space over the magnet array for a variety of values of the MFC dimensions to identify the magnetic force F=η(B·grad(B)) on the magnetically labeled non-target cells relative to the magnet array, where η is a constant which comprises the number of magnetic particles attached to a cell, the volume of the magnetic particles and the difference in susceptibility between the magnetic particles and the fluid (see, e.g., FIG. 11); c) performing a high-resolution numerical simulation of the fluid flow in 3-dimensional (3D) free space within the MSD channel to determine the fluidic force on cells for a given flow rate; d) performing particle tracking simulations of cells within the MSD channel based on the sum of both fluidic force (c) and magnetic force (b) (see, e.g., FIG. 12); and e) using the particle simulations to predict final cell density on the channel wall (see, e.g., FIG. 10) and iterating or performing an optimization on the MFC position to achieve the desired cell capture profile in (a). In certain embodiments, performing step b) comprises performing said high-resolution numerical simulation using a variety of values of the dimensions of the gap from the MFC to the channel.

In some embodiments, the system may include: a processor programmed to control a microfluidic magnetic device, as described herein. The processor may be programmed to perform steps of the computer implemented method comprising: (a) injecting the MNPs and one or more negative selection binding agents into a DLD channel or curved microchannel through an inlet; (b) injecting a fluid sample into the DLD channel or curved microchannel through an inlet; (c) adjusting fluidic flow to a target steady rate; (d) controlling amount of time the cells are incubated with the MNPs and the one or more negative selection binding agents in the incubation tubing; and (e) controlling flow of the cells through the MSD while applying an exponentially increasing magnetophoretic gradient to capture the magnetically labeled non-target cells from the blood sample using the MSD.

The method can be implemented in digital electronic circuitry, or in computer software, firmware, or hardware. The disclosed and other embodiments can be implemented as one or more computer program products, i.e., one or more modules of computer program instructions encoded on a computer readable medium for execution by, or to control the operation of, a data processing apparatus. The computer readable medium can be a machine-readable storage device, a machine-readable storage substrate, a memory device, a composition of matter effecting a machine-readable propagated signal, or any combination thereof.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

In a further aspect, the system for performing the computer implemented method, as described, may include a processor, a storage component (i.e., memory), a display component, and other components typically present in general purpose computers. In some embodiments, the processor is provided by a computer or handheld device (e.g., a cell phone or tablet). The storage component stores information accessible by the processor, including instructions that may be executed by the processor and data that may be retrieved, manipulated or stored by the processor.

The storage component includes instructions. For example, the storage component includes instructions for determining positioning of a fluidic conduit and the magnetic flux concentrator relative to a single magnet of a plurality of magnets of the magnetophoretic separation device, as described herein. The computer processor is coupled to the storage component and configured to execute the instructions stored in the storage component in order to model magnetic field and magnetophoretic forces at different locations relative to the magnetic array; and model fluidic forces on the cells and the magnetic nanoparticles present within the channel; and identify a set of spatial coordinates to position the fluidic conduit and the magnetic flux concentrator relative to the plurality of magnets to achieve a target captured cell density along the fluidic conduit using one or more algorithms, as described herein. The display component displays information regarding the coordinates for positioning of a fluidic conduit (e.g., fluidic tubing) in a channel of the MSD and/or coordinates for the positioning of a magnetic flux concentrator relative to the plurality of magnets of the MSD.

The storage component may be of any type capable of storing information accessible by the processor, such as a hard-drive, memory card, ROM, RAM, DVD, CD-ROM, USB Flash drive, write-capable, and read-only memories. The processor may be any well-known processor, such as processors from Intel Corporation. Alternatively, the processor may be a dedicated controller such as an ASIC.

The instructions may be any set of instructions to be executed directly (such as machine code) or indirectly (such as scripts) by the processor. In that regard, the terms “instructions,” “steps” and “programs” may be used interchangeably herein. The instructions may be stored in object code form for direct processing by the processor, or in any other computer language including scripts or collections of independent source code modules that are interpreted on demand or compiled in advance.

Data may be retrieved, stored or modified by the processor in accordance with the instructions. For instance, although the system is not limited by any particular data structure, the data may be stored in computer registers, in a relational database as a table having a plurality of different fields and records, XML documents, or flat files. The data may also be formatted in any computer-readable format such as, but not limited to, binary values, ASCII or Unicode. Moreover, the data may comprise any information sufficient to identify the relevant information, such as numbers, descriptive text, proprietary codes, pointers, references to data stored in other memories (including other network locations) or information which is used by a function to calculate the relevant data.

In certain embodiments, the processor and storage component may comprise multiple processors and storage components that may or may not be stored within the same physical housing. For example, some of the instructions and data may be stored on removable CD-ROM and others within a read-only computer chip. Some or all of the instructions and data may be stored in a location physically remote from, yet still accessible by, the processor. Similarly, the processor may comprise a collection of processors which may or may not operate in parallel. In some embodiments, a hardware accelerator is used. In some embodiments, imaging data is divided into chunks for processing by a plurality of graphics processing units (GPUs), field-programmable gate arrays (FPGAs), or tensor processing units (TPUs).

In some embodiments, the method can be performed using a cloud computing system. In these embodiments, data files and the programming can be exported to a cloud computer, which runs the program, and returns an output to the user.

Components of systems for carrying out the presently disclosed methods are further described in the examples below.

### Utility

The devices and methods disclosed herein can be used to isolate any cell of interest from a fluid sample. Devices and diagnostic methods for allergy testing are also disclosed herein, which are useful for screening candidate allergens for potential allergic responses and accurately predict the severity of allergic reactions in individuals. Furthermore, the devices and methods can be conveniently used for allergy screening at a hospital, for example, in a doctor's office or an emergency room, or at home. Patients who experience adverse allergic reactions to allergens can be quickly screened to determine the source of the problem such that repeated exposure to allergens can be avoided.

The devices and diagnostic methods described herein are also useful in monitoring the progress of treatment of patients undergoing food immunotherapy. Although a subject may need to completely avoid foods that cause adverse allergic reactions, increased tolerance of a problem food may be achieved by administering slowly small but steadily increasing doses of the problem food until the patient is desensitized to it. Allergy testing can be performed using the devices and methods described herein to determine whether food immunotherapy has been effective in achieving food allergy desensitization.

Candidate allergens that can be tested by the methods described herein include any type of allergen, including ingested allergens, inhaled allergens, occupational allergens, environmental allergens, or any other substance that triggers a harmful immune reaction resulting in activation of basophils. Blood samples may be tested with various candidate allergens to test for various types of allergies such as, but not limited to, a food allergy (e.g., strawberries and other fruits and vegetables, peanuts, soy, and other legumes, walnuts and other treenuts, shellfish and other seafood, milk and other dairy products, wheat and other grains, and eggs), an animal allergy (e.g., cat), a cockroach allergy, a tick allergy, a dust mite allergy, an insect sting allergy (e.g. (bee, wasp, and others), a latex allergy, a medication allergy (e.g., penicillin, carboplatin), mold and fungi allergies (e.g., Alternaria alternata, Aspergillus and others), a pollen allergy (e.g., ragweed, Bermuda grass, Russian thistle, oak, rye, and others), and a metal allergy.

Sets of allergens may be tested including, without limitation, environmental allergens, e.g. pollens; insect allergens, e.g. bee venom, spider venom, etc.; food allergens, e.g. fish, shellfish (shrimp, crab, lobster, oyster, scallops), soy, strawberries, tree nuts (walnut, hazel/filbert, cashew, pistachio, Brazil, pine nut, almond), peanuts, milk, egg protein, etc.; drug allergens, e.g. penicillin, etc. For example, a panel of insect allergens may comprise epitopes from a plurality of insect venoms, e.g. Myrmecia pilosula; Apis mellifera bee venom phospholipase A2 (PLA2) and antigen 5S; phospholipases from the yellow jacket Vespula maculifrons and white-faced hornet Dolichovespula maculate, etc. A panel of pollen allergens may comprise epitopes from a plurality of plants, e.g. birch pollen, ragweed pollen, Parol (the major allergen of Parietaria officinalis) and the cross-reactive allergen Parjl (from Parietaria judaica), and other atmospheric pollens including Olea europaea, Artemisia sp., gramineae, etc.

The compositions and methods of the invention are applicable to a variety of allergens, including food allergens; environmental allergens; animal allergens; etc. Allergens include, e.g. pollens; insect allergens, e.g. bee venom, spider venom, etc.; food allergens, e.g. fish, shellfish (shrimp, crab, lobster, oyster, scallops), soy, strawberries, tree nuts (walnut, hazel/filbert, cashew, pistachio, Brazil, pine nut, almond), peanuts, milk, egg protein, etc.; drug allergens, e.g. penicillin, etc. Insect venoms include, e.g. Myrmecia pilosula; Apis mellifera bee venom phospholipase A2 (PLA2) and antigen 5S; phospholipases from the yellow jacket Vespula maculifrons and white-faced hornet Dolichovespula maculate, etc. Pollen allergens may comprise epitopes from a plurality of plants, e.g. birch pollen, ragweed pollen, Parol (the major allergen of Parietaria officinalis) and the cross-reactive allergen Parjl (from Parietaria judaica), and other atmospheric pollens including Olea europaea, Artemisia sp., gramineae, etc.

Allergens of interest for analysis by the methods of the invention include, without limitation, allergens associated with anaphylaxis, which include food allergens, insect allergens and drug allergens. Allergens known to be associated with anaphylaxis include food allergens: peanuts, tree nuts, fish, shellfish, cow's milk, soy, and eggs; insect allergens, particularly from stinging insects, e.g. honeybees, fire ants, yellow jackets, yellow hornets and paper wasps; drugs: B-lactams; nonsteroidal anti-inflammatory drugs (NSAIDs); biologic modifiers, e.g. cetuximab, infliximab and omalizumab. Suitable panels comprising an epitope array from one or more of these allergens are provided.

Other allergens of interest are those responsible for allergic dermatitis caused by blood sucking arthropods, e.g. Diptera, including mosquitos (Anopheles sp., Aedes sp., Culiseta sp., Culex sp.); flies (Phlebotomus sp., Culicoides sp.) particularly black flies, deer flies and biting midges; ticks (Dermacenter sp., Ornithodoros sp., Otobius sp.); fleas, e.g. the order Siphonaptera, including the genera Xenopsylla, Pulex and Ctenocephalides felis.

Some specific allergens of interest include egg proteins, which can be provided in a panel of egg protein epitopes from one, two, three, four or more different egg proteins; in combination with a panel of food allergens; etc. Egg allergens include, e.g. Ovomucoid (Gal d1), 210 aa protein; Genbank Accession: P01005.1; Ovalbumin (Gal d2), AltName: Egg Albumin, Plakalbumin, 386 aa protein; Genbank Accession: P01012.2; Ovotransferin (Gal d3), AltName: Conalbumin, Serumtransferrin, 705 aa protein; Genbank Accession: P02789.2; Lysozyme C (Gal d4), AltName: 1,4-beta-N-acetylmuramidase C, 147 aa protein; Genbank Accession: P00698.1; Alpha-livetin (Gal d5), 615 aa protein, Genbank Accession: P19121.2; etc.

Some specific allergens of interest include peanut proteins, which can be provided in a panel of peanut protein epitopes from one, two, three, four or more different peanut proteins; in combination with a panel of food allergens; etc. Peanut allergens include, e.g. Ara h1 (614 a.a); Ara h2.0101 or Ara h2.0201/Conglutin-7/2S albumin (172 a.a) k Ara h3 Glycinin Uniprot O82580; Ara h4 Glycinin seed storage protein Uniprot 5712199; Ara h5 profilin Uniprot Q9SQ19); Ara h6 Conglutin homolog uniprot 5923742; Ara h7 Conglutin homolog uniprot 5923742; Ara h8 Ara h8.0101/PR-10 protein uniprot Q6VT83; Ara h8.0201/PR-10 protein uniprot BOYIU5; Ara h9 Ara h LTP isoallergen precursor; etc.

Some specific allergens of interest include cow's milk proteins, which can be provided in a panel of milk protein epitopes from one, two, three, four or more different milk proteins; in combination with a panel of food allergens; etc. Milk allergens include, Bos Casein βeta-A1 (Uniprot 162797/162805/162931/459292); βeta-A3 Uniprot 459292); αS1 Uniprot 162929; αS2; Kappa uniprot 162811, Beta-lactoglobulin; Alpha-lactalbumin, etc.

Some specific allergens of interest include shellfish proteins, which can be provided in a panel of shellfish protein epitopes from one, two, three, four or more different shellfish proteins; in combination with a panel of food allergens; etc. Shellfish allergens include shrimp tropomyosin: Cra c1 accession D7F1J4; Lit v1 accession B4YAH6; Met e1 accession Q25456; Pan b1 accession P86704, Pen a1 accession AAZ76743.1, Pen i1 tropomyosin; shrimp arginine kinase: Cra c2 accession D7F1J5; Lit v2, accession Q004B5; Pen m2, accession E7CGC2; shrimp sarcoplasmic calcium-binding protein: Cra c4, accession D7F1P9; Pen e4; Pen m4 accession E7CGC4; Lit v4 accession C7A639; shrimp myosin light chain: Art fr 5, accession A7L499; Cra c5, accession D7F1Q1; Lit v3, accession B7SNI3, Pen m3, accession E1A683; shrimp troponin C: Pen m6, accession E7CGC5; Cra c6, accession D7F1Q2; triosephosphate isomerase Cra c8, accession D7F1Q0. Crab allergens include tropomyosin: Cha f 1, accession Q9N2R3; Chi 01, accession A2V735; TPM_CHIOP; Can p 1; Eri i 1; Par c 1; Por s 1; Por tr 1; Ran ra 1; Scy o 1, accession A1KYZ3; Scy pa 1; Scy s 1, accession A7L5V2; arginine kinase Chi o2, accession C9EIP1; Scy s 2; Lim p 2 accession P51541; troponin: Chi o 6, accession P86910; Sarcoplasmic calcium-binding protein: Chi o 4, accession P86909. Lobster allergens include tropomyosin: Hom a1 accession 044119.1; Pan s 1 accession O61379.1; arginine kinase: Hom g 2 accession P14208.4; myosin light chain 2: Hom a3 accession EH115965; troponin C: om a6 accession P29291; sarcoplasmic calcium-bindingL Hom a4. Crayfish allergens include tropomyosin: Pro cl i accession ACN87223.1; sarcoplasmic calcium-binding protein: Pon 14 accession P05946; troponin I: Pon 17 accession P05547. Krill allergens include tropomypsin: Eup p1 accession BAF76431.1; Eup s 1 accession dbj|BAF95205.1. Mollusk allergens include tropomyosin: Hel as 1 accession CAB38044; Hal a 1 accession AAP85231.1; Tod p 1 accession Q9BLG0.3; hemocyanin: Meg C accession CAG28309.2; paramyosin: Hal di accession BAJ61596.1; Myt g accession BAA36517.1; Oct v; Tur c; Hal r.

Some specific allergens of interest include soy proteins, which can be provided in a panel of soy protein epitopes from one, two, three, four or more different soy proteins; in combination with a panel of food allergens; etc. Soy allergens include Gly m 5 Glycine Beta-conglycinin accession CAA35691.1; Gly m 5 Glycine Beta-conglycinin accession AAA33947.1; Gly m 5 Glycine Beta-conglycinin accession AAB01374.1; Gly m 5 Glycine Beta-conglycinin accession AAB23463.1; Glycine Gly m 1 accession AAB34755.1; Glycine Gly m 1 accession ABA54898.1; Glycine Gly m 3 accession CAA11755.1; Glycine Gly m 3 accession O65809.1; Glycine Gly m 3 accession ABU97472.1; Glycine Gly m 4 accession P26987.1; Glycine Gly m 8 2s albumin accession AAD09630.1; Glycine Gly m Bd 28K accession BAB21619.1; Glycine Gly m Bd 28K accession ACD36976.1; Glycine Gly m Bd 28K accession ACD36975.1; Glycine Gly m Bd 28K accession ACD36974.1; Glycine Gly m Bd 28K accession ACD36978.1; Glycine Gly m Bd accession P22895.1; Glycine Gly m Bd accession AAB09252.1; Glycine Gly m Bd accession BAA25899.1; Glycine Glycinin G1 accession CAA26723.1; Glycine Glycinin G1 accession CAA33215.1; Glycine Glycinin G2 accession CAA26575.1; Glycine Glycinin G2 accession CAA33216.1; Glycine Glycinin G3 accession CAA33217.1; Glycine Glycinin G4 accession CAA37044.1; Glycine Glycinin G5 accession AAA33964.1; Glycine Glycinin G5 accession AAA33965.1; Glycine Major Gly 50 kDa allergen accession P82947.1; Glycine Trypsin inhibitor accession AAB23464.1; Glycine Trypsin inhibitor accession AAB23482.1; Glycine Trypsin inhibitor accession AAB23483.1; Glycine Trypsin inhibitor accession CAA56343.1; Glycine Glycinin G4 accession CAA60533.1; Glycine Glycinin G5 accession CAA55977.1.

Some specific allergens of interest include tree nut proteins, which can be provided in a panel of tree nut protein epitopes from one, two, three, four or more different tree nut proteins; in combination with a panel of food allergens; etc. Tree nut allergens include Almonds: Pru du 3/Pru du 3.0101 (123 aa) Accession: ACN11576s/GI: 223667948; Pru du 4 Accession: AAL91664/GI: 24473798; Pru du 5/Pru du 5.0101 Accession: ABH03379/GI: 111013714; Pru du 6/Amandin Accession: ADN39440/GI: 307159112; Chain A, Amandin (531 aa)-Accession: 3EHK_A/GI: 258588247; Chain B, Amandin (531 aa)-Accession: 3EHK_B/GI: 258588248; Chain C, Amandin (531 aa)-Accession: 3EHK_C/GI: 258588249; Prunin 2 precursor/Pru du 6.0201, Accession: ADN39441/GI: 307159114; Putative Pru du 6 Accession: AGR27935/GI: 523916668. Walnuts: Jug n1 Accession: AAM54365/GI: 31321942 or AAB41308/GI: 1794252; Jug n2 Vicillin seed storage protein (481 aa)-Accession: AAM54366/GI: 31321944; Jug r1 Albumin Seed Storage, Accession: AAB41308/GI: 1794252; Jug r2 Accession: AAF18269/GI: 6580762; Jug r3; Jug r4 Accession: AAW29810/GI: 56788031. Cashews: Ana o1 accession: AAM73730/GI: 21914823; Ana o2 Accession: AAN76862/GI: 25991543; Ana o3 Accession: AAL916651/GI: 24473800. Chestnuts: Cas s1 Accession: CAD10374/GI: 16555781; Cas s5 Accession: Q42428/GI: 75282355; Chitinase isoform 2 Accession: ADN39439/GI: 307159110; Endochitinase Accession: P29137/GI: 116301; Cas s8; Cas s9 Accession: CAE46905/GI: 46359518. Pecans: Car i1 Accession: AAO32314/GI: 28207731; Car i4 Accession: ABW86978/GI: 158998780; Accession: ABW86979/GI: 158998782. Hazelnuts: Cor a 1 Accession: CAA50327/GI: 22688; Cor a 1.0102 (161 aa)-Accession: CAA50328/GI: 22690; Cor a 1.0103 (161 aa)-Accession: CAA50325/GI: 22684; Cor a 1.0104 (161 aa)-Accession: CAA50326/GI: 22686; Cor a 1.0201 (161 aa)-Accession: CAA96548/GI: 1321731; Cor a 1.0301 (161 aa)-Accession: CAA96549/GI: 1321733; Cor a 1.0401 (161 aa)-Accession: AAD4840/GI: 5726304; Cor a 1.0402 (161 aa)-Accession: AAG40329/GI: 11762102; Cor a 1.0403 (161 aa)-Accession: AAG40330/GI: 11762104; Cor a 1.0404 (161 aa)-Accession: AAG40331/GI: 11762106 Cor a 10 Accession: CAC14168/GI: 10944737; Cor a 11 Accession: AAL86739/GI: 19338630; Cor a 12/oleosin Accession: AAO67349/GI: 49617323; Cor a 13/oleosin Accession: AAO65960/GI: 29170509; a Cor 14/2S albumin Accession: ACO56333/GI: 226437844; Cor a2 Accession: AAK01235/GI: 12659206; Cor a8 Accession: AAK28533/GI: 13507262; Cor a9 Accession: AAL73404/GI: 18479082. Pistachio: Pis v1 Accession: ABG73108/GI: 110349081; Pis v2 Accession: ABG73109/GI: 110349083; Pis v2.0201/11S globulin precursor ABG73110/GI: 110349085, Accession: ABU42022/GI: 156001070; Pis v3; Accession: ABO36677/GI: 133711974; Pis v4 Accession: ABR29644/GI: 149786150; Pis v5 Accession: ACB55490/GI: 171853010.

Some specific allergens of interest include wheat proteins, which can be provided in a panel of wheat protein epitopes from one, two, three, four or more different wheat proteins; in combination with a panel of food allergens; etc. Wheat allergens include Profilin (Tri a 12); Tri a 12.0101 accession P49232; Tri a 12.0102 accession P49233; Tri a 12.0103 accession P49234; Tri a 12.0104 accession B6EF35; Tri a 14.0201 accession D2T2K2; Tri a 15.0101 accession D2TGC3; Tri a 18.0101 accession P10968; Tri a 19.0101; Tri a 21.0101 accession D2T2K3; Tri a 25.0101 accession Q9LDX4; Tri a 26.0101 accession P10388; Tri a 26.0201 accession Q45R38; Tri a 27.0101 accession Q7Y1Z2; Tri a 28.0101 accession Q4WOV7; Tri a 29.0101 accession C7C4X0; Tri a 29.0201 accession D2TGC2; Tri a 30.0101 accession P17314; Tri a 31.0101 accession Q9FS79; Tri a 32.0101 accession Q6W8Q2; Tri a 33.0101 accession Q9ST57; Tri a 34.0101 accession C7C4X1; Tri a 35.0101 accession D2TE72; Tri a 36.0101 accession 335331566; Tri a 37.0101 accession Q9TOP1; Tri a 39.0101 accession J7QW61.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-93 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below.

1. A magnetophoretic separation device (MSD) comprising:
a) a single magnet or a magnetic array comprising a plurality of magnets;
b) a scaffold comprising a slot to secure the single magnet or the magnetic array and one or more channels, wherein each channel comprises one or more fluidic conduits connected to one or more outlets; and
c) one or more magnetic flux concentrators in proximity to the one or more fluidic conduits, wherein spacing of the one or more magnetic flux concentrators relative to the single magnet or the magnetic array and the one or more fluidic conduits is selected to produce a target magnetophoretic gradient profile, wherein magnetophoretic force varies along the length of the one or more fluidic conduits.

2. The MSD of aspect 1, wherein the magnetic array is a Halbach array.

3. The MSD of aspect 2, wherein the Halbach array is a cylindrical Halbach array comprising a plurality of ring magnets or a linear Halbach array comprising a plurality of bar magnets.

4. The MSD of aspect 1, wherein the magnetic array comprises multiple magnets with opposing polarities, multiple magnets with aligned polarities, a grid of magnets with alternating polarities, or self-assembling magnetic colloids.

5. The MSD of any one of aspects 1-4, wherein the magnetic array comprises at least five magnets.

6. The MSD of any one of aspects 1-5, wherein the magnets are permanent magnets or electromagnets.

7. The MSD of aspect 6, wherein the permanent magnets are neodymium magnets.

8. The MSD of any one of aspects 1-7, wherein the one or more fluidic conduits are provided by fluidic tubing positioned adjacent to the magnetic array or the magnetic flux concentrator.

9. The MSD of aspect 8, wherein the fluidic tubing has an inner diameter sufficiently small that magnetic flux from the one or more magnetic flux concentrators reaches the center axis of the fluidic tubing.

10. The MSD of aspect 9, wherein the inner diameter is sufficiently large to retain captured non-target cells without obstructing the one or more types of target cells from flowing through the fluidic tubing.

11. The MSD of any one of aspects 1-7, wherein the one or more fluidic conduits are machined, embossed, rastered, or etched into the one or more channels.

12. The MSD of any one of aspects 1-11, wherein the one or more channels are planar, multi-layered, or three-dimensional.

13. The MSD of any one of aspects 1-12, wherein each fluidic conduit provides a straight fluid path, a curved fluid path, or a serpentine fluid path.

14. The MSD of aspect 13, wherein the fluid path revolves around the plurality of ring magnets.

15. The MSD of any one of aspects 13, wherein the fluidic path runs parallel to the plurality of bar magnets.

16. The MSD of any one of aspects 1-15, wherein the fluidic path is bifurcated to direct fluid flow to regions of concentrated magnetic flux.

17. The MSD of any one of aspects 1-16, wherein the channel width ranges from 0.01 mm to 100 mm.

18. The MSD of any one of aspects 1-17, wherein the channel height ranges from 0.01 mm to 100 mm.

19. The MSD of any one of aspects 1-18, wherein the channel length ranges from 1 mm to 1000 mm.

20. The MSD of any one of aspects 1-19, wherein the one or more fluidic conduits have a square, rectangular, circular, elliptical, or polygonal cross-sectional shape.

21. The MSD of any one of aspects 1-20, wherein the one or more channels further comprise one or more fluidic elements that focus all or a subset of cells from a fluid sample towards an area of higher magnetic force field.

22. The MSD of aspect 21, wherein one or more fluidic elements comprise a constriction, an expansion, a vane, or a sheath flow device, or a combination thereof.

23. The MSD of any one of aspects 1-22, further comprising one or more gutters connected to the one or more fluidic conduits.

24. The MSD of any one of aspects 1-23, wherein the one or more fluidic conduits further comprise a staggered herringbone mixer, dean vortices, parallel lamination of fluid streamlines, micropillars, or steps.

25. The MSD of any one of aspects 1-24, wherein the magnetic flux concentrator produces a magnetic flux density ranging from 0.1 tesla to 10 tesla.

26. The MSD of any one of aspects 1-25, wherein the magnetic flux concentrator has a relative magnetic permeability ranging from 100 to 100,000.

27. The MSD of any one of aspects 1-26, wherein the magnetic flux concentrator comprises a fluidic channel comprising a ferrofluid or a self-assembled magnetic colloid.

28. The MSD of any one of aspects 1-27, wherein the magnetic flux concentrator radius can be adjusted to tune peak magnitude of the magnetic force on cells in the one or more fluidic conduits.

29. The MSD of any one of aspects 1-28, wherein the magnetophoretic force increases exponentially along the length of the one or more fluidic conduits.

30. The MSD of any one of aspects 1-29, wherein the MSD comprises two or more channels, each channel comprising a separate fluidic conduit connected to a different outlet, wherein each channel can be used to separate a different type of target cell from non-target cells from a different fluid sample.

31. The MSD of aspect 30, wherein the two or more channels are parallel to each other.

32. The MSD of aspect 31, wherein the MSD comprises two or more magnetic flux concentrators, wherein the magnetic flux concentrators are parallel to the two or more channels.

33. The MSD of any one of aspects 30-32, wherein each outlet is connected to a separate collection reservoir or a common collection reservoir.

34. The MSD of any one of aspects 30-33, wherein each channel or a subset of channels shares the same magnetic array and magnetic flux concentrator, or each channel has a different magnetic array and magnetic flux concentrator.

35. The MSD of any one of aspects 1-34, further comprising a plurality of pumps.

36. The MSD of aspect 35, wherein the pumps are syringe pumps, diaphragm pumps, peristaltic pumps or piston pumps, or any combination thereof.

37. The MSD of aspect 35, wherein the pumps are positive displacement pumps or pressure generating pumps.

38. The MSD of any one of aspects 1-37, further comprising a plurality of sensors that monitor flow rate or pressure, or a combination thereof.

39. The MSD of aspect 38, wherein the pumps and sensors are configured to maintain the flow rate through the magnetic separation device in a range from 0.01 mL/hr to 20,000 L/hr.

40. The MSD of any one of aspects 1-39, wherein the one or more fluidic conduits share magnetic arrays, magnetic flux concentrators, pumps, mixing elements, or flow through cytometers, or any combination thereof.

41. The MSD of any one of aspects 1-40, wherein each magnetic flux concentrator comprises a plurality of ferromagnetic wires, sheets, self-assembling colloids, or other configuration of ferromagnetic material running alongside one or more of the fluidic conduits, wherein spacing of the wires, sheets, self-assembling colloids, or other configuration of ferromagnetic material relative to the magnetic array and the one or more of the fluidic conduits is selected to produce the target magnetophoretic gradient profile.

42. The MSD of any one of aspects 1-41, wherein the MSD is contained in a microfluidic cartridge comprising a first assembly comprising the one or more channels and the one or more magnetic flux concentrators; and a second assembly comprising the magnetic array.

43. A microfluidic magnetic device for isolation of one or more types of target cells from a fluid sample comprising a heterogenous population of cells, the device comprising:
a) one or more inlets to receive the fluid sample;
b) one or more mixers connected to the one or more inlets, wherein the one or more mixers mix magnetic nanoparticles (MNPs) and one or more negative selection binding agents with the cells, wherein the one or more negative selection binding agents selectively bind to one or more surface markers on non-target cells, wherein the one or more surface markers are not present on the one or more types of target cells;
c) one or more incubation tubings connected to the one or more mixers, wherein the cells are incubated with the MNPs and the one or more negative selection binding agents in the one or more incubation tubings, wherein the non-target cells that are bound to the one or more negative selection binding agents are selectively magnetically labeled with the MNPs;
d) the MSD of any one of aspects 1-39, wherein the one or more channels of the MSD are connected to the one or more incubation tubings, wherein the MSD captures the magnetically labeled non-target cells and outputs the one or more types of target cells; and
e) one or more outlets connected to the MSD, wherein the one or more types of target cells are expelled from the one or more outlets.

44. The microfluidic magnetic device of aspect 43, further comprising one or more deterministic lateral displacement (DLD) channels or curved microchannels that sort cells based on size, wherein each DLD channel or curved microchannel is positioned between one of the inlets and one of the mixers, wherein an input end of each DLD channel or curved microchannel is connected to one of the inlets and an output end of each DLD channel or microchannel is connected to one of the mixers.

45. The microfluidic magnetic device of aspect 44, wherein the one or more DLD channels and one or more mixers are contained on a microfluidic chip comprising a first layer comprising the mixers and a second layer comprising the DLD channels.

46. The microfluidic magnetic device of aspect 45, wherein the first layer comprising the one or more mixers is above the second layer comprising the one or more DLD channels.

47. The microfluidic magnetic device of any one of aspects 43-46, wherein the one or more DLD channels further comprise a coating to reduce cell adhesion to the DLD channels.

48. The microfluidic magnetic device of aspect 47, wherein the coating comprises a non-ionic surfactant.

49. The microfluidic magnetic device of any one of aspects 43-48, further comprising a means for applying a secondary Dean flow velocity field or a hydrophoretic force to the one or more curved microchannels when fluid passes through the curved microchannels.

50. The microfluidic magnetic device of any one of aspects 44-49, wherein the one or more curved microchannels vary in width along the length of the curved microchannels.

51. The microfluidic magnetic device of any one of aspects 44-50, wherein the one or more curved microchannels further comprise perpendicular or angled steps.

52. The microfluidic magnetic device of any one of aspects 44-51, wherein the one or more types of target cells comprise an immune cell, a blood cell, a stem cell, or a cancer cell, or any combination thereof.

53. The microfluidic magnetic device of aspect 52, wherein the immune cell is a basophil, a neutrophil, an eosinophil, a mast cell, a monocyte, a dendritic cell, a macrophage, a T cell, a B cell, or a natural killer cell.

54. The microfluidic magnetic device of aspect 53, wherein the basophil is an activated basophil or an unactivated basophil.

55. The microfluidic magnetic device of any one of aspects 43-54, wherein the fluid sample is a blood sample.

56. The microfluidic magnetic device of aspect 55, wherein the one or more DLD channels or curved microchannels separate white blood cells from red blood cells.

57. The microfluidic magnetic device of any one of aspects 43-56, wherein the one or more mixers comprise one or more Herringbone-grooved serpentine channels.

58. The microfluidic magnetic device of aspect 57, wherein each Herringbone-grooved serpentine channel has dimensions of about 200 μm in width and about 70 μm in height.

59. The microfluidic magnetic device of aspect 57 or 58, wherein each Herringbone-grooved serpentine channel comprises a groove having a height of about 30 μm.

60. The microfluidic magnetic device of any one of aspects 43-59, wherein the one or more negative selection binding agents are an antibody, an antibody mimetic, a peptoid, an aptamer, or a ligand.

61. The microfluidic magnetic device of any one of aspects 43-60, wherein the microfluidic magnetic device further comprises a flow-through microfluidic cytometer capable of measuring numbers and properties of the one or more types of target cells, wherein the flow-through microfluidic cytometer is fluidically connected to the magnetophoretic separation device.

62. The microfluidic magnetic device of any one of aspects 43-61, further comprising a plurality of pumps.

63. The microfluidic magnetic device of aspect 62, wherein the pumps are syringe pumps, diaphragm pumps, peristaltic pumps or piston pumps, or any combination thereof.

64. The microfluidic magnetic device of aspect 62, wherein the pumps are positive displacement pumps or pressure generating pumps.

65. The microfluidic magnetic device of any one of aspects 43-64, further comprising a plurality of sensors that monitor flow rate or pressure, or a combination thereof.

66. The microfluidic magnetic device of aspect 65, wherein the pumps and sensors are configured to maintain the flow rate through the magnetic separation device in a range from 0.01 mL/hr to 20,000 L/hr.

67. The microfluidic magnetic device of any one of aspects 43-66, where the microfluidic magnetic device comprises a plurality of the MSDs to allow processing of parallel samples.

68. A method of using the microfluidic magnetic device of any one of aspects 43-67, the method comprising:
a) introducing the fluid sample comprising the heterogenous population of cells into the one or more inlets;
b) mixing the magnetic nanoparticles (MNPs) and the one or more negative selection binding agents with the cells using the one or more mixers, wherein the one or more negative selection binding agents selectively bind to the one or more surface markers on the non-target cells, wherein the one or more surface markers are not present on the one or more types of target cells;
c) incubating the cells with the MNPs and the one or more negative selection binding agents in the one or more incubation tubings, wherein the non-target cells that are bound to the one or more negative selection binding agents are selectively magnetically labeled with the MNPs;
d) applying a magnetophoretic gradient to capture the magnetically labeled non-target cells from the fluid sample using the MSD; and
e) collecting the one or more types of target cells expelled from the one or more outlets.

69. The method of aspect 68, wherein the microfluidic magnetic device comprises one or more deterministic lateral displacement (DLD) channels or a curved microchannels, wherein the method further comprises flowing the fluid sample into the one or more DLD channels or curved microchannels, wherein the one or more DLD channels or curved microchannels separate cells based on size.

70. The method of aspect 68 or 69, wherein the fluid sample is a blood sample.

71. The method of aspect 70, wherein the one or more DLD channels or curved microchannels separate white blood cells from red blood cells.

72. The method of aspect 70, further comprising separating white blood cells from red blood cells by density gradient centrifugation, wherein said introducing the fluid sample into the one or more inlets comprises introducing a fluid sample comprising the separated white blood cells into the one or more inlets.

73. The method of aspect 70, further comprising sorting cells by using an acoustic radiation force or dielectrophoretic activity in an electric field prior to said mixing the nanoparticles (MNPs) and the one or more negative selection binding agents with the cells.

74. A microfluidic magnetic device for isolation of one or more types of target cells from a blood sample, the device comprising:
a) one or more inlets to receive a blood sample;
b) one or more deterministic lateral displacement (DLD) channels connected to the inlets, wherein the one or more DLD channels separate white blood cells from red blood cells in the blood sample;
c) one or more mixers connected to the one or more DLD channels, wherein the DLD channels mix magnetic nanoparticles (MNPs) and one or more negative selection binding agents with the separated white blood cells, wherein the one or more negative selection binding agents selectively bind to one or more surface markers on non-target cells, wherein the one or more surface markers are not present on the one or more types of target cells;
d) one or more incubation tubings connected to the one or more mixers, wherein the white blood cells are incubated with the MNPs and the one or more negative selection binding agents in the one or more incubation tubings, wherein the non-target cells that are bound to the one or more negative selection binding agents are selectively magnetically labeled with the MNPs;
e) a magnetophoretic separation device (MSD) connected to the one or more incubation tubings, wherein the MSD applies an exponentially increasing magnetophoretic gradient to capture the magnetically labeled non-target cells; and
f) one or more outlets connected to the MSD, wherein the one or more types of target cells are expelled from the one or more outlets.

75. The microfluidic magnetic device of aspect 74, wherein the one or more types of target cells comprise an activated basophil or an unactivated basophil.

76. The microfluidic magnetic device of aspect 74 or 75, wherein the one or more negative selection binding agents are antibodies, antibody mimetics, aptamers, peptoids, or ligands.

77. The microfluidic magnetic device of aspect 76, wherein the antibodies comprise an anti-HLA-DR antibody, an anti-CD2 antibody, an anti-CD3 antibody, an anti-CD14 antibody, an anti-CD15 antibody, an anti-CD16 antibody, an anti-CD19 antibody, an anti-CD20 antibody, an anti-CD24 antibody, an anti-CD34 antibody, an anti-CD36 antibody, an anti-CD45RA antibody, an anti-CD56 antibody, an anti-CD66b antibody, or an anti-glycophorin A antibody, or any combination thereof.

78. A kit comprising the MSD of any one of aspects 1-42 or the microfluidic magnetic device of any one of aspects 43-67 and 74-77 and instructions for isolating one or more types of target cells from a fluid sample.

79. The kit of aspect 78, further comprising one or more negative selection binding agents that selectively bind to one or more surface markers on non-target cells, wherein the surface marker is not present on the one or more types of target cells.

80. The kit of aspect 79, wherein the one or more negative selection binding agents are antibodies, antibody mimetics, aptamers, peptoids, or ligands.

81. The kit of any one of aspects 78-80, further comprising magnetic nanoparticles.

82. The kit of any one of aspects 78-81, wherein the one or more types of target cells comprise a basophil, a neutrophil, an eosinophil, a mast cell, a monocyte, a dendritic cell, a macrophage, a T cell, a B cell, a natural killer cell, or a stem cell, or any combination thereof.

83. The kit of aspect 82, wherein the basophil is an activated basophil or an unactivated basophil.

84. A computer implemented method for controlling the microfluidic magnetic device of any one of aspects 43-67, the computer performing steps comprising:
a) injecting the MNPs and the one or more negative selection binding agents into the one or more DLD channels or curved microchannels through the one or more inlets;
b) injecting the fluid sample into the one or more DLD channels or curved microchannels through the one or more inlets;
c) adjusting fluidic flow to a target steady rate;
d) controlling amount of time the cells are incubated with the MNPs and the one or more negative selection binding agents in the one or more incubation tubings; and
e) controlling flow of the cells through the MSD while applying an increasing magnetophoretic gradient to capture the magnetically labeled non-target cells from the fluid sample using the MSD.

85. A non-transitory computer-readable medium comprising program instructions that, when executed by a processor in a computer, causes the processor to perform the method of aspect 84.

86. A system for isolating one or more types of target cells from a blood sample, the system comprising:
a) a microfluidic magnetic device for isolation of the one or more target cells from the blood sample, the device comprising:
   (i) one or more inlets to receive a blood sample;
   (ii) one or more deterministic lateral displacement (DLD) channels or curved microchannels connected to the one or more inlets, wherein the one or more DLD channels or curved microchannels separate white blood cells from red blood cells in the blood sample;
   (iii) one or more mixers connected to the one or more DLD channels or curved microchannels, wherein the one or more mixers mix magnetic nanoparticles (MNPs) and one or more negative selection binding agents with the separated white blood cells, wherein the one or more negative selection binding agents selectively bind to one or more surface markers on non-target cells, wherein the one or more surface markers are not present on the one or more types of target cells;
   (iv) one or more incubation tubings connected to the one or more mixers, wherein the white blood cells are incubated with the MNPs and the one or more negative selection binding agents in the one or more incubation tubings, wherein the non-target cells that are bound to the one or more negative selection binding agents are selectively magnetically labeled with the MNPs;
   (v) a magnetophoretic separation device (MSD) connected to the one or more incubation tubings, wherein the MSD applies an exponentially increasing magnetophoretic gradient to capture the magnetically labeled non-target cells; and
   (vi) one or more outlets connected to the MSD, wherein the one or more types of target cells are expelled from the one or more outlets; and
b) a processor programmed to control the microfluidic magnetic device according to the computer implemented method of aspect 84.

87. A computer implemented method for determining positioning of one or more fluidic conduits and one or more magnetic flux concentrators (MFC) relative to the plurality of magnets of the magnetophoretic separation device (MSD) of any one of aspects 1-42, the computer performing steps comprising:
modeling magnetic field and magnetophoretic forces at different locations relative to a magnetic array; and
identifying one or more sets of spatial coordinates to position one or more fluidic conduits and one or more magnetic flux concentrators relative to the plurality of magnets to achieve a target exponential magnetophoretic force profile along the fluidic conduit.

88. The computer implemented method of aspect 87, wherein the computer performs steps comprising:
a) generating a target mathematical profile of max ∥(∇·B̅)B̅∥, or another metric for a magnetic force field, as a function of the path of the fluidic conduit smax ∥(∇·B̅)B̅∥ [symbol] metric is set according to the highest value omax ∥(∇·B̅)B̅∥ [symbol] ;
b) identifying different combinations of zₚₐₜₕ and d_{MFC} that could generate a target magnetophoretic force profile along the path of the fluidic conduit by plotting max ∥(∇·B̅)B̅∥ obtained from a parametric sweep on a surface as a function of zₚₐₜₕ and d_{MFC}, and where zₚₐₜₕ can be defined in any direction relative to the magnetic array, magnetic flux concentrator, or any other logical definition;
c) identifying candidate paths (q) on the max ∥(∇·B̅)B̅∥ surface that would generate a target profile f(s);
d) translating the candidate parametric paths into physical path positions;
e) performing a high-resolution numerical simulation of the magnetic field in 3-dimensional (3D) free space over the magnetic array to identify a set of (x, y, z) points in the simulation that satisfy the following two criteria: 1) d\vec{B}(x, y, z)/dy≈0, and 2) ∥B_{3D,yz}(x, y, z)∥≈∥\vec{B}_{2D}(zₚₐₜₕ)∥;
f) displaying sets of 3D candidate paths translated from parameter space that achieve the target magnetophoretic force profile;
g) evaluating 3D candidate paths using a cost function to select for a path that minimizes the following functions:

$${α}_{1} = max ( \frac{dz}{dx} ) - 1 , \qquad 1 )$$

which selects for paths with gradual slopes for the fluidic conduit to follow in the xz-plane,

$${α}_{2} = {\int }_{0}^{L} {d}_{MFC} dx - \frac{0.45 L}{2} , \qquad 2 )$$

which selects for MFC paths with gradually varying d_{MFC} from 450 to 0 μm across the total length L of the path to facilitate the 3D printing and the threading of the MFC. 3) Σⱼ₌₁^{N}|∥\vec{B}_{2D,j}∥−∥\vec{B}_{3D,yz,j}∥|, which selects for paths that can best match ∥\vec{B}_{3D,yz}(x, y, z)∥ with ∥\vec{B}_{2D}(zₚₐₜₕ)∥ across all N points in the path; and
h) selecting the path e with the minimum cost function

$${min}_{θ} \frac{1}{3} {\sum }_{i = 1}^{3} {[ {α}_{i} ( θ ) ]}^{2}$$

as the final position of the fluidic conduit and the MFC to construct the MSD.

89. A computer implemented method for determining positioning of a fluidic conduit and a magnetic flux concentrator (MFC) relative to the plurality of magnets of the magnetophoretic separation device (MSD) of any one of aspects 1-42, the computer performing steps comprising:
modeling magnetic field and magnetophoretic forces at different locations relative to the magnetic array; and
modeling fluidic forces on the cells and the magnetic nanoparticles present within the channel; and
identifying a set of spatial coordinates to position the fluidic conduit and the magnetic flux concentrator relative to the plurality of magnets to achieve a target captured cell density along the fluidic conduit.

90. The computer implemented method of aspect 89, wherein the computer performs steps comprising:
a) generating a target mathematical cell capture profile for capture of magnetically labeled non-target cells;
b) performing a high-resolution numerical simulation of the magnetic field in 3-dimensional (3D) free space over the magnet array for a variety of values of the MFC dimensions to identify the magnetic force F=η(B·grad(B)) on the magnetically labeled non-target cells relative to the magnet array, where η is a constant which comprises the number of magnetic particles attached to a cell, the volume of the magnetic particles and the difference in susceptibility between the magnetic particles and the fluid;
c) performing a high-resolution numerical simulation of the fluid flow in 3-dimensional (3D) free space within the MSD channel to determine the fluidic force on cells for a given flow rate;
d) performing particle tracking simulations of cells within the MSD channel based on the sum of both fluidic force (c) and magnetic force (b); and
e) using the particle simulations to predict final cell density on the channel wall and iterating or performing an optimization on the MFC position to achieve the desired cell capture profile in (a)

91. The computer implemented method of aspect 90, wherein said performing step b) comprises performing said high-resolution numerical simulation using a variety of values of the dimensions of the gap from the MFC to the channel.

92. The computer implemented method of any one of aspects 87-91, wherein the fluidic conduit is provided by fluidic tubing positioned adjacent to the magnetic array or the magnetic flux concentrator.

93. A non-transitory computer-readable medium comprising program instructions that, when executed by a processor in a computer, causes the processor to perform the method of any one of aspects 87-92.

It will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

### Example 1

A Microfluidic Magnetic Separation Device with an Exponential Magnetophoretic Gradient for Cell Isolation from Whole Blood

### 1. Introduction

Microfluidics has emerged as a promising technology for the rapid and precise separation and isolation of cells, in part due to its ability to control the application of hydrodynamic, magnetic, and other forces precisely to control the path of cells in the microchannel. It is also possible to integrate and automate different steps from a conventional assay to minimize the need for manual manipulation. Extensive work exist for separating WBC from RBCs^{44,45} and capturing specific cell types (e.g., T cells⁴⁶ or circulating tumor cells (CTCs)⁴⁷⁻⁴⁹) in microfluidic systems. However, few have reported on basophil isolation. Although some approaches such as those using acoustophoresis were capable of segmenting monocyte, granulocyte, and lymphocyte populations with >83% purity for each subpopulation, they were unable to further distinguish granulocyte subtypes (i.e., basophils, neutrophils, eosinophils).⁵⁰

Since basophils share similar size, density, and other physical characteristics with other granulocytes,⁵¹ immunoselection is the most reliable way of isolating basophils. Microfluidic magnetic-activated cell sorting (MACS) is commonly used for on-chip immunoselection.⁵² The primary advantage of MACS is the high specificity with which cell types can be targeted using negatively or positively selecting antibodies. On-chip MACS also allows the implementation of spatially uniform or varying magnetic fields to sort magnetically-tagged cells.^{52,53} This capability enables the deflection⁵⁴ or capture⁵⁵ of magnetically-tagged cells with varying levels of magnetic susceptibility in regions of different magnetic strengths. For example, in the on-chip MACS platform for the immunomagnetic negative selection of CTCs, two regions with different magnitudes of magnetic flux were used to first deflect highly magnetic cells (>10 magnetic beads per cell) and then deflect lowly magnetic cells (>1 bead per cell) to waste outlets. While this design demonstrated high purity and recovery of CTCs from a large volume of sample, density gradient centrifugation was needed to enrich WBCs from whole blood before feeding into the MACS device.⁵⁴ This method also required sequential manual, off-chip magnetic labeling (i.e., the product from one MACS run was re-labeled at a higher magnetic bead concentration and re-run through the device), and was highly dependent on precise alignment of the channels with the external magnetic field. To our knowledge, no work has demonstrated on-chip MACS isolation of basophils from whole blood with a purity and recovery that is comparable to immunomagnetic negative selection approaches in bulk. Furthermore, no work has demonstrated a model-driven MACS design pipeline to produce the desired magnetic flux for efficient cell isolation.

In this work, we describe the design and development of an integrated microfluidic basophil isolation device (i-BID) to perform the immunomagnetic negative selection of basophils from whole blood. We choose to perform negative selection instead of positive selection to maintain basophils in their native, unlabeled state. The device consists of deterministic lateral displacement (DLD) channels to enrich WBCs from whole blood, a mixer to mix magnetic nanoparticles (MNP) and negative selection antibody (NSAb) with enriched WBCs, and a magnetophoretic separation device (MSD) to deplete non-basophils (FIG. 1A). The key innovation lies in the design of the MSD that applies an exponentially increasing magnetophoretic gradient to non-basophils to capture them gradually on the inner walls of polyethylene tubing as the cells flow through. This approach contrasts current methods that have a single region with constant magnetic force or discrete regions of differing magnetic forces.^{52,54,55} A gradual initial increase in magnetophoretic force prevents an abrupt, excessive buildup of captured non-basophils and unbound MNP which could lead to clogging and non-specific capture of basophils. The steep increase in magnetophoretic force at the end of the MSD serves to capture non-basophils with low numbers of MNP and any unbound MNP. The MSD uses steel wires as magnetic flux concentrators (MFCs) and a 3D printed scaffold to sweep the path of the tubing across an array of magnets, which consists of five magnets arranged in a Halbach configuration. The spacing of the MFC wires and the tubing relative to the Halbach array is guided by a target exponential magnetophoretic gradient profile and numerical simulations of the resultant magnetic fields across the tubing and the MFC. Between each run, a new tubing is inserted into the MSD without the need for fabricating new microchannels or re-aligning the flow path with the magnets. Our design thus presents significant advantage over existing microfluidic MACS devices which require microfabrication of ferromagnetic structures and their careful alignment with the microchannel.^{52,53} With the i-BID, we achieve a mean purity of >93.5% (range 85.5-97.0%) and a mean recovery of >96.5% (range 91.3-100%; ˜1600 basophils) from 100 μL of blood, which is too small a volume to process using bulk isolation methods. The entire process takes ˜10 min from the loading of whole blood to the retrieval of isolated basophils, which remain viable and inactivated.

### 2. Experimental Design

### Blood Sample Preparation

Blood was collected via finger prick into heparin tube from 5 healthy donors. For all experiments, blood samples were kept on ice for at most 3 hours prior to injection into the microfluidic device.

For experiments characterizing the fully integrated i-BID, we mixed 100-200 μL of unwashed heparinized whole blood with 1-2 μL of 500 mM EDTA (final EDTA concentration ˜5 mM) prior to injection into the i-BID. Heparin served to inhibit free thrombin to reduce platelet aggregation, and EDTA served to chelate calcium to mitigate the formation of clots in the DLD channels.⁵⁶

For a subset of experiments characterizing the operating conditions of the standalone MSD, we enriched WBCs from 500-600 μL of blood at a time in DLD channels. Because this subset of experiments was not aimed at rapid, fully integrated basophil isolation with the i-BID, we washed the blood samples to dilute the clotting factors (e.g., fibrinogen and thrombin) and mitigate clogging in the DLD channels. We had observed that injecting whole blood directly led to considerable build-up of cells at the DLD entrance. The wash was performed by diluting blood ˜3000× in 5 mM EDTA in PBS (Ca/Mg), centrifuging the suspension (500 G, 5 min), and aspirating the supernatant for a final volume and hematocrit approximately equivalent to that of the starting whole blood sample.

For all experiments, we added stains to the blood samples to identify basophils in flow cytometry to characterize their purity and recovery after the microfluidic operation. Per 100 μL of blood, we added 3 μL of anti-CD123-APC (clone 7G3, BD Bioscience), 4 μL of anti-CD45-PE (clone HI30, BioLegend), and 4 μL of anti-CD193-PerCP/Cy5.5 (clone 5E8, BioLegend), and incubated the sample for ˜18 minutes on ice. Following the stain incubation and prior to injection into the microchannels, we added 22 μL of DLD running buffer (2% FBS, 1 mM EDTA in PBS) for each 100 μL of blood to obtain a final sample volume of 133 μL (blood concentration of ˜75% v/v). This step was performed to reduce the chance of clogging.

### Fabrication and Design of the Deterministic Lateral Displacement (DLD) Device and the Mixer

Both the DLD and the mixer were fabricated in poly(dimethylsiloxane) (PDMS) using soft lithography. The master molds were fabricated in SU8-2050 using transparency masks (CAD/Art Services, Inc.) and a Quintel Q-4000 mask aligner. The DLD/mixer microfluidic chip comprised of two layers of PDMS. The bottom layer contained the DLD channels, and the top layer contained the mixer channel. The two-layer design was necessary for parallelizing the DLD channels as described below.

For the DLD, we adapted the design from Feng et al.⁵⁷ To improve the throughput and to reduce the rate of clogging at the entrance of the DLD arrays, we used four parallel DLD channels. Each DLD channel had a height of 50 μm, a pillar diameter of 22 μm, a gap of 13 μm, a row shift fraction of 1/30, and an approximate critical diameter of ˜3.6 μm (see FIG. 1B). 57-59 We coated the DLD channels with Pluronic F68 (3% w/v, Alfa Aesar J66087 Poloxamer 188) to reduce the incidence of cell adhesion and the likelihood of clogging.⁶⁰ Pluronic F68 was left in the channels for at least one hour, after which it was thoroughly flushed with the DLD running buffer (2% FBS, 1 mM EDTA in PBS) prior to use.

After the DLD, MNP and NSAb were introduced and mixed with the WBCs from the DLD using a mixer consisting of a Herringbone-grooved serpentine channel.⁶¹ The MNP and NSAb were obtained from a basophil isolation kit supplied by STEMCELL (catalog #17969). The NSAb mixture negatively selected for basophils with a mixture of antibodies targeting non-basophil surface antigens (e.g., CD2, CD3, CD14, CD15, CD16, CD19, CD24, CD34, CD36, CD45RA, CD56, glycophorin A).⁶² The mixer channels were 200 μm wide and 70 μm tall with groove heights of 30 μm. In our experiments, the flow running through the device was between 3-9 mL/hr, corresponding to a Reynolds number between ˜5 to 20, well within the mixer design's operating conditions.⁶¹ MNP and NSAb reagents were injected at a rate necessary to maintain a target MNP/NSAb: WBC ratio of 50 μL:1 mL. Their injection rate was thus a function of the expected flow rate of the enriched WBC suspension exiting the DLD channels. For example, to achieve this target ratio for a WBC suspension exiting the DLD stage at 3 mL/hr, we injected MNP/NSAb at 0.15 mL/hr. After use, we flushed the channels with 10% bleach. If all debris was visually removed, the channels were re-coated with F68 and reused.

### Fabrication of the Magnetic Separation Device (MSD)

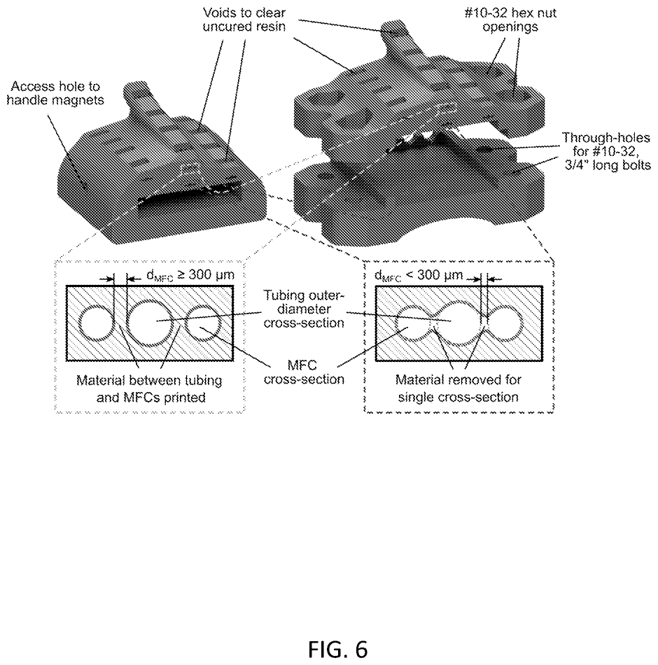

The Halbach array consisted of five N52 bar magnets (1.5″ by 0.75″ by 0.75″, Super Magnet Man, catalog #Rect1526). We used a stereolithography 3D printer (Form2, Formlabs) to print the MSD scaffold in Grey Pro resin with a layer resolution of 50 μm. The scaffold consisted of a slot to secure the Halbach array, passage tunnels to position a fluidic tubing (medical grade polyethylene tubing, 762 μm inner diameter, Scientific Commodities, catalog #BB31695) and steel wire MFC (low carbon 1008 steel, 0.029″ dia., McMaster, catalog #8870K15), and openings along the passages to facilitate the removal of uncured resin. We chose a tubing with an inner diameter of 762 μm because its cross-sectional area and the corresponding internal surface area were sufficiently large to retain captured non-basophils and unbound MNP without obstructing the flow of basophils, while it was sufficiently small so that the magnetic flux from the MFC could reach the center axis of the tubing. As described in the section 3.2, the design called for gap distances between the tubing and the MFC (d_{MFC}) smaller than the minimum feature size of the From2 printer. At d_{MFC}<300 μm, we combined the tubing and MFC passage cross sections to avoid imperfections from attempting to print below the Form2 resolution (FIG. 6).

Flow Control of the Integrated Basophil Isolation Device (i-BID)

In I all experiments, we loaded the blood cells and the basophil isolation reagents in medical grade polyethylene tubing, which were connected to syringes filled with DLD running buffer. To prevent the cell suspensions and reagents from back flowing into the syringes, we used air plugs (5-10 mm long) to separate them from the DLD running buffer which filled the rest of the tubing and syringes. While the air plugs could introduce a source of compressibility into the i-BID, their effects were not observed during steady-state operation.

Blood flow was actuated with a 1 m1 syringe (HSW Norm-ject) and MNP/NSAb flows were each actuated with 250 μL glass syringes (SGE Analytical Science) to prevent syringe deformation under pressure. DLD buffer injected into the DLD/mixer and isolated basophils withdrawn from the MSD were actuated with 10 mL syringes (BD Biosciences).

We wrote a custom control program in Python to operate the syringe pumps (Cheymx Inc.). The control code facilitated loading the blood sample, ramping up the flow rate to a constant steady state, and stepping through a series of user-defined operation modes (i.e., injecting blood, flushing the DLD channels, and passing the cells through the MSD). A slight latency in writing and running commands to the pumps across USB serial ports was expected and could lead to a positive pressure feedback loop. To avoid this lag, we implemented threading in Python to instantiate multiple processes in parallel and update all pumps simultaneously.

Prior to injecting blood, we initiated the injection of MNP/NSAb and the withdrawal from the DLD/mixer outlet while gradually stepping up the DLD buffer flow until steady state was reached. We fixed the DLD buffer flow rate (Q_{buffer}) at 5× the flow rate at which the blood sample was injected (Q_{blood}) into the DLD. The flow rates are chosen to keep the average velocities the same in all channels feeding into and out of the DLD arrays. We fixed the flow rate of the mixed WBC-MNP/NSAb suspension (Qₒᵤₜ) by withdrawing from the outlet of the MSD at a rate equal to the sum of the injection rates of MNP, NSAb, and the WBC outlet flow rate (see FIG. 1A). The RBC waste outlet tubing was submerged in the fluid of the waste collection container to avoid pressure fluctuations due to dripping and to maintain a constant pressure at the waste outlet. After the entire volume of blood was injected into the DLD/mixer channels and right before the air plug entered the channel, we advanced the Python control program to the next step to halt blood injection, flushed the DLD channels with DLD running buffer, and ramped down the injection of MNP/NSAb as the WBC-MNP/NSAb suspension continued through MSD.

### Characterization of the Performance of the Standalone MSD

In a subset of experiments to evaluate the effectiveness of the MSD in isolating basophils as a function of flow rate, we enriched WBCs using the same DLD channels that comprised the i-BID. Basophil recovery from the DLD was >95% for all blood injection flow rates tested (2-8 mL/hr). Aliquots of the DLD product (100 μL, with ˜2-3×10⁵ WBCs) were mixed with 5 μL of NSAb and 5 μL of MNP in a 5 mL round-bottom tube with a gentle shake. After 5 min of incubation at room temperature, we injected the mixture into the standalone MSD with a syringe pump in withdrawal mode from 3-18 mL/hr.

For a subset of experiments, we tested the operation of the standalone MSD by injecting the enriched WBCs manually from a syringe. Briefly, we asked 5 participants to withdraw the WBC-MNP/NSAb suspension from the outlet of the MSD by hand using a 1 m1 syringe. Participants were instructed to steadily withdraw the sample through the MSD in 2-3 minutes resulting in approximate flow rates of 6-8 mL/hr.

Comparison with Commercial Magnetic Column

We compared the performance of the MSD, i-BID, and manual MSD to that of the STEMCELL EasySep magnetic column (catalog #18000). We followed the same process detailed above for enriching WBCs from blood and adding MNP/NSAb to 100 μL WBC suspension. After 5 min of incubation with MNP/NSAb, we added 3 mL of DLD running buffer and placed the tube in the magnetic column. We allowed 5 minutes for the removal of magnetically tagged cells following the EasySep protocol. After that, with the tube remaining in the magnetic column, the suspension containing purified basophils was poured into a new 5 mL tube. The cell suspension was then centrifuged at 500 G for 5 min to remove the excess buffer and prepare the cells for flow cytometry.

### Characterization of the Purity and Recovery of Basophils

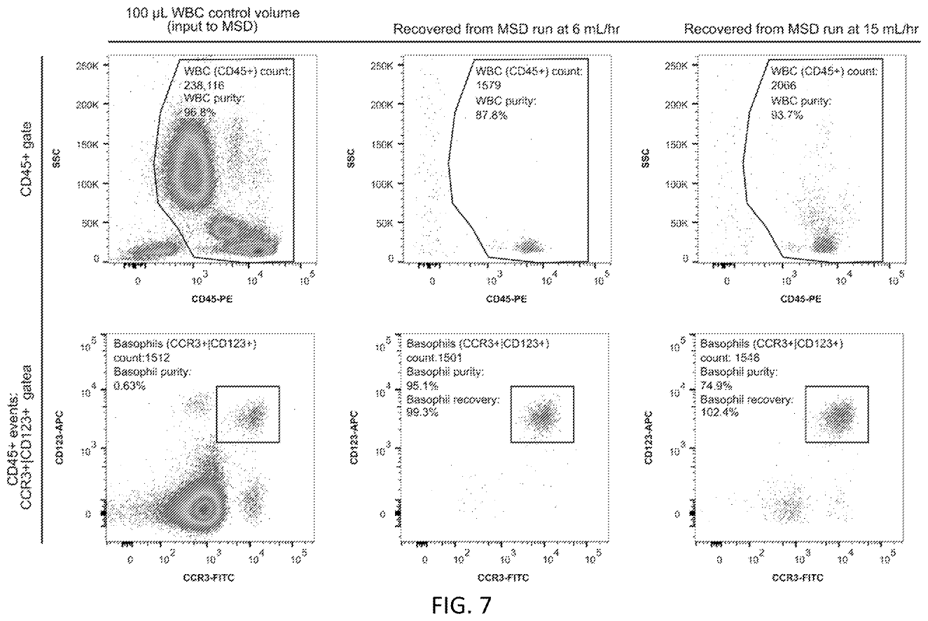

We quantified the purity and recovery of basophils from the product of our device using flow cytometry (BD FACScan, Cytek Biosciences). Purity was quantified by performing flow cytometry on the MSD product to determine the percentage of events that were SSC^{low}/CD45⁺/CD123⁺/CCR3⁺ (FIG. 7). We followed the convention established by the STEMCELL basophil isolation kit protocol in which purity is determined as the percent of basophils within cells expressing the leukocyte common antigen (CD45).⁴⁰

Recovery was quantified by dividing the count of basophils in the MSD product by the count of basophils in the control described as follows. In our recovery quantification, we assumed a homogenous distribution of basophils in WBC suspensions and in blood. For experiments characterizing the performance of the fully integrated i-BID, recovery was approximated by dividing the count of basophils produced by the i-BID by the count of basophils in a control sample of blood with volume equivalent to that injected into the i-BID. This control was stained for CD123, CCR3, and CD45, and WBCs were enriched by lysing RBCs. For experiments characterizing the operating conditions of the standalone MSD, enriched WBC suspensions obtained by DLD were split into 5-6 aliquots each with a volume of 100 μL. One of these aliquots was set aside as the control to determine the expected number of basophils in 100 μL of the WBC suspension.

In all experiments, we also collected the RBC waste from the DLD channel, lysed the RBCs, and injected any remaining WBCs through the flow cytometer to assess the recovery of basophils from the DLD.

To quantify the purity and recovery of basophils using the commercial magnetic column, we followed the same procedure as above.

### Characterization of Basophil Activation Status

We performed experiments to examine whether the isolated basophils exhibit any undesired activation in our on-chip isolation process, and whether they could still undergo activation in response to a stimulus. For these experiments, the surface marker stains were not added until after isolated basophils were recovered and subjected to stimulus. Basophils, when recovered, were suspended in DLD running which contained EDTA. Because EDTA chelates calcium ions and basophil activation is a calcium and magnesium dependent process,²⁹ we resuspended the basophils in RPMI with 1 uM of CaCl_{2)(aq)} and MgCl_{2(aq)} prior to subjecting them to any stimulus. Following this step, 100 μL of basophil suspensions were mixed with 100 μL of RPMI or anti-IgE (2 μg/mL) dissolved in 100 μL of RPMI. After incubation for 30 minutes at 37° C. in 5% CO₂, the activation was halted by adding 1 mL of cold 2.5 mM EDTA in PBS (Ca/Mg) to each sample. The samples were centrifuged at 500 G for 5 min at 4° C. The supernatant was aspirated, and the pellet was resuspended by vortex mixing. We then added 2 μL per stain per sample for identifying basophils (i.e., anti-CD123-APC and anti-HLA-DR-PE (clone L243, BD Biosciences) and for identifying activated basophils (i.e., anti-CD63-FITC (clone H5C6, BD Biosciences)). The cells were incubated with the stains for 20 min on ice followed by a wash with 3 mL of a stain buffer (2 mM EDTA and 0.5% BSA in PBS). Cells were centrifuged at 500 G for 5 min at 4° C. The supernatant was aspirated, and the pellet was resuspended in stain buffer for flow cytometry.

For measuring the activation status of basophils from the control sample of whole blood, we used the same procedure for measuring activation in purified basophil and included an RBC lysis step after the stain. We lysed RBCs by adding 4 mL of 1×RBC lysis buffer (Biolegend, catalog #420301) to the resuspended pellet. We incubated the lysis buffer with the cells for 15-20 minutes in the dark at room temperature, and centrifuged the lysed suspension at 800 G for 10 min at 4° C. The supernatant was aspirated, and the cells were resuspended in 3 mL of stain buffer before another centrifugation at 500 G for 5 min at 4° C. to collect WBCs for flow cytometry.

### 3. Results and Discussions

### 3.1 Overall Process Flow of the Integrated Basophil Isolation Device

FIG. 1A shows the process flow of our integrated basophil isolation device (i-BID). It consisted of four stages: 1) DLD channels to enrich WBCs from whole blood, 2) serpentine channels with Herringbone grooves to mix MNP/NSAb with enriched WBCs, 3) a section of tubing whose length determined the incubation time for tagging non-basophils with MNP, 4) the MSD for removing non-basophils from the suspension. Here, we selected a DLD channel to separate WBC from RBC because of the high purity and recovery of WBC and the compatibility of its operating flow rates with downstream mixing of MNP/NSAb and basophil isolation in the MSD. We verified that the recovery of basophils was >95% from the DLD for blood injection rates tested (2-8 mL/hr). Following the DLD, the enriched WBCs were mixed with MNP and NSAb in a mixer consisting of a serpentine channel with Herringbone grooves.⁶¹ Dean and helical flows induced by the channel's serpentine bends and Herringbone grooves, respectively, promoted efficient mixing of MNP and NSAb with WBCs and increased the incidence of binding events between cells and MNP (see insets in FIG. 1B). We showed qualitatively that separate streamlines were well mixed before the exit of the mixer. In transit to the MSD, the MNP/NSAb and WBC were incubated in the incubation tubing for 1 to 3 min, which translated to a tubing length of 9 to 27 cm, respectively, when operating at a blood injection flow rate of 3 mL/hr. The incubation tubing was fed to the inlet of the MSD to trap magnetically tagged non-basophils and excess MNP (FIG. 1C). The outlet of the MSD contained enriched basophils, which were collected to assess purity, recovery, and activation status.

### 3.2 Design and Operation of the MSD

The concentration of basophils in peripheral blood is approximately 1% of WBCs which represents ˜10 cells/μL.² In contrast to the isolation of CTCs which are present at much lower concentrations (less than a few cells per mL), basophil isolation requires a smaller volume of blood than CTC isolation does. This difference allowed us to design a MACS device based on capture mode instead of deflection mode because the magnetically tagged non-basophils and unbound MNP could be sequestered adequately on the inner walls of a microtubing. The capture mode also offered a simple means to integrate basophil isolation with downstream on-chip processes. There is no need to match the fluidic resistance of the product and the waste outlets, which would be necessary in devices operating in deflection mode.

FIG. 1C shows a schematic diagram and a photograph of the MSD consisting of a 3D-printed scaffold housing a Halbach array, a polyethylene tubing threaded over the Halbach array in four passes, and steel wires acting as magnetic flux concentrators (MFC) positioned parallel to the tubing on its left and right. The Halbach array comprised 5 magnets arranged in a Halbach configuration. We chose to use a Halbach array because the resulting magnetic field was stronger, more far-reaching, and symmetric about the center plane (parallel to the xz-plane) of the array than other possible configurations of the 5 magnets. We chose the positions of the tubing and the MFC in order to achieve an exponentially increasing magnetophoretic force along the length of the tubing, i.e., the path of the cells, to capture magnetically tagged cells and unbound MNP from the flow gradually. The gradual initial increase in the magnetophoretic force prevented abrupt accumulation of magnetically tagged cells and MNP which could lead to clogging and undesired trapping of basophils. The steep increase of magnetic flux gradient at the end of the MSD helped to remove non-basophils with low magnetic susceptibility and ensured that any unbound MNP and magnetic debris did not escape the MSD and contaminate the product of purified basophils. In principle, other magnetophoretic force profiles that are slowly increasing should also work. Here we selected an exponential profile since it was compatible with the Halbach array consisting of 5 magnets and 4 passes across the magnetic field, but the design pipeline can accommodate other target profiles.

Four passes were chosen to maximize the length of tubing that could span the 5 magnets while avoiding physical overlap between adjacent passes. Based on the polarity of the magnets in the Halbach array, we chose to have the tubing sweep along the long axes of the magnets because it resulted in a more uniform magnetic force along the tubing, and therefore facilitated achieving the target exponential profile, than to have the tubing traverse across all magnets which would result in abrupt changes in the magnetic force. Since the tubing was sandwiched by the MFC laterally, positioning the tubing over magnets 1, 3, and 5 (see FIG. 2A), where d\vec{B}/dy≈0, allowed the magnetic flux to be concentrated inside the tubing by the MFC. We chose to have passes 1 and 4 traverse over magnet 3 because the far-reaching magnetic field over magnet 3 could accommodate low magnetic forces in pass 1 and high magnetic forces in pass 4. Despite the adjacency of passes 1 and 4, no interference occurred in the magnetic field due to the proximity of the MFC in passes 1 and 4. To prevent tubing intersection over magnet 3, it followed that passes 2 and 3 must span magnets 1 and 5, respectively.

We used numerical simulation in COMSOL (AC/DC module) to model the magnetic field and magnetophoretic forces at different locations relative to the Halbach array. The goal of the simulations was to identify a set of spatial coordinates to position the tubing and the MFC relative to the magnets to achieve the target exponential magnetophoretic force profile along the path of the tubing. These spatial coordinates were then used to inform a 3D printed model of the MSD scaffold to hold the tubing, the MFCs, and the magnets. To save computation cost, our approach started with a 2D simulation of the magnetic fields in the yz plane (FIG. 2A) when varying the relative distance between the tubing and the magnets (zₚₐₜₕ), and between the tubing at the MFC (d_{MFC}). After that, we identified combinations of zₚₐₜₕ and d_{MFC} that could give an exponential magnetophoretic force profile, and then translated these distances to actual 3D coordinates for the tubing and the MFC.

The governing equation for the force that a magnetic field exhibits on a magnetic particle is given in Eq. 1,^{52,53}

$${\vec{F}}_{m} \frac{V △χ}{{μ}_{0}} ( ∇ \cdot \vec{B} ) \vec{B} \qquad ( Eq . 1 )$$

where V is the volume of the particle, Ax is the difference in magnetic susceptibilities between the particle and the fluid, μ₀ is the magnetic permeability in a vacuum (4 π×10⁻⁷ Tm/A), \vec{B} is the magnetic flux density, and ∇·\vec{B} is the gradient of the magnetic flux density. We designed the MSD around the (∇·B̅)B̅ term, also referred to in this discussion as the magnetophoretic gradient, since the magnetophoretic force scales directly with this term and VΔχ/μ₀ is constant for a given size and material of the particles.

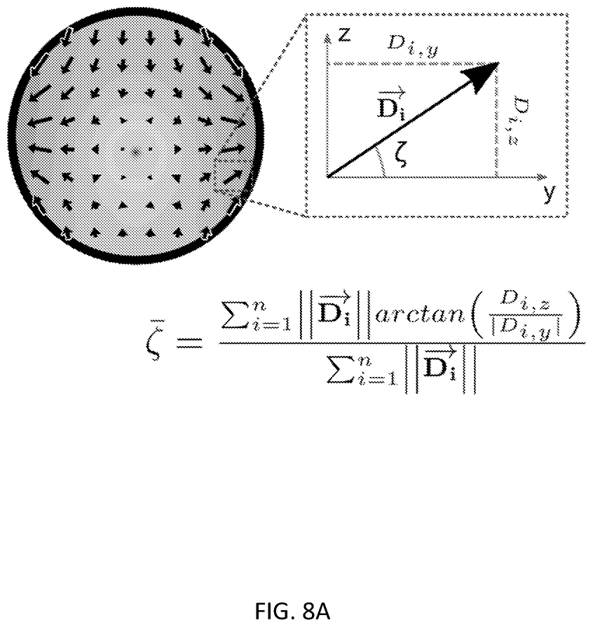

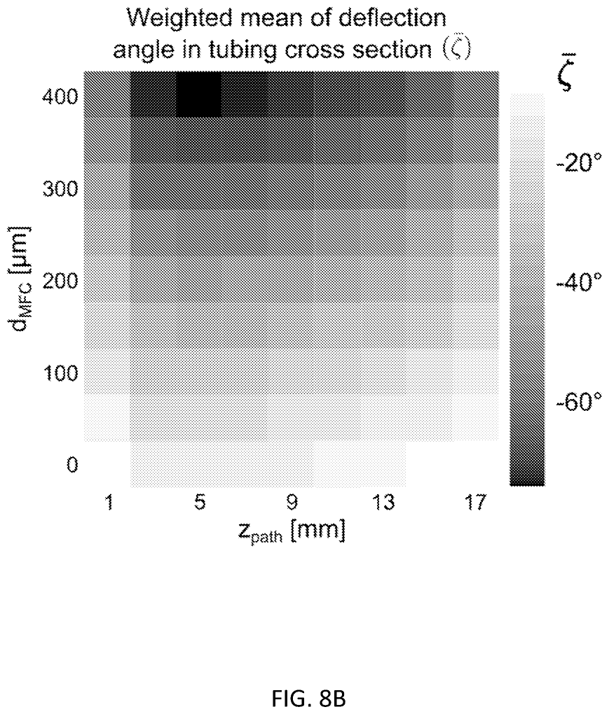

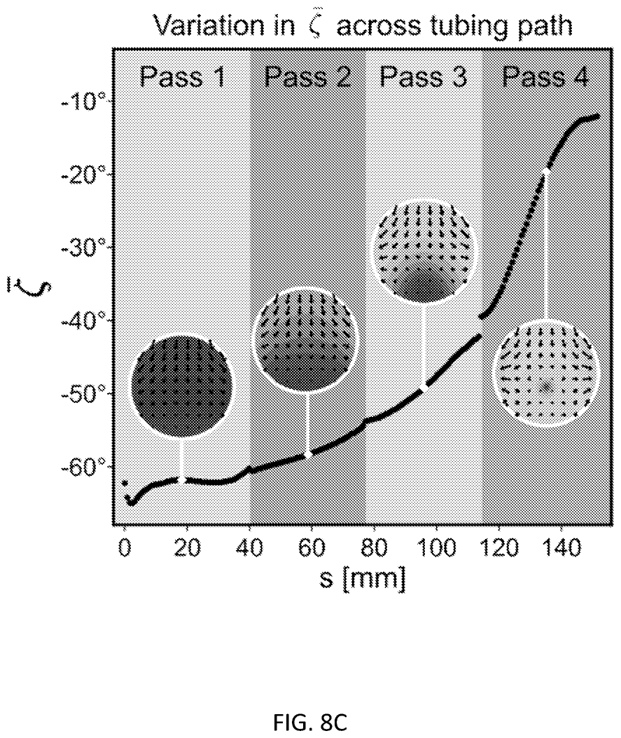

To vary (∇·B̅)B̅, we varied zₚₐₜₕ and d_{MFC}. We performed a parametric sweep to model the magnetic flux concentration by the MFC as a function of zₚₐₜₕ and d_{MFC} in 2D at the midplane of the Halbach array above magnet 3 where d\vec{B}/dy≈0 (section A-A in FIG. 2A). FIG. 2B shows the distribution of the magnitude and the direction of the magnetophoretic gradient inside the tubing for a few combinations of zₚₐₜₕ and d_{MFC}. As expected, the smallest zₚₐₜₕ (=1 mm) and d_{MFC} (=0 mm) yielded the highest maximum magnitude of the magnetophoretic gradient within the cross section of the tubing (i.e., max ∥(∇·B̅)B̅∥˜900 T²/m), corresponding to the strongest magnetophoretic force. When d_{MFC} was small, max ∥(∇·B̅)B̅∥) occurred close to the left and the right rims of the tubing. This distribution was expected since the MFC were positioned to the left and the right of the tubing (FIG. 8). When d_{MFC} was large, the strongest magnetophoretic force occurred close to the bottom of the tubing, since the effect of the MFC was weakened compared to the Halbach array. For most combinations of zₚₐₜₕ and d_{MFC}, both lateral and vertical-pointing forces were present, thereby allowing the MNP-tagged cells to be captured across a greater area of the inner wall of the tubing compared with other MACS devices with strictly lateral or vertical-pointing magnetic forces. In addition, the “dead zone” with a zero net magnetic force did not maintain a constant position along the tubing, and therefore, the tagged cells in all streamlines within the tubing would experience a force to be captured.

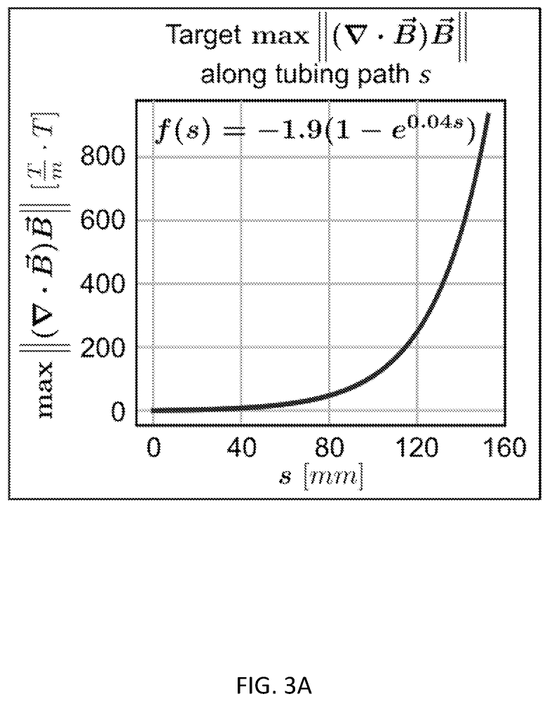

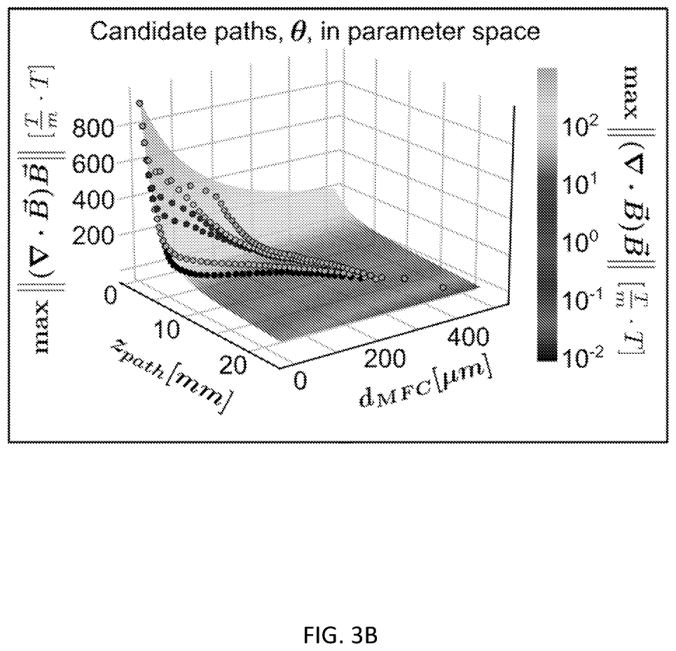

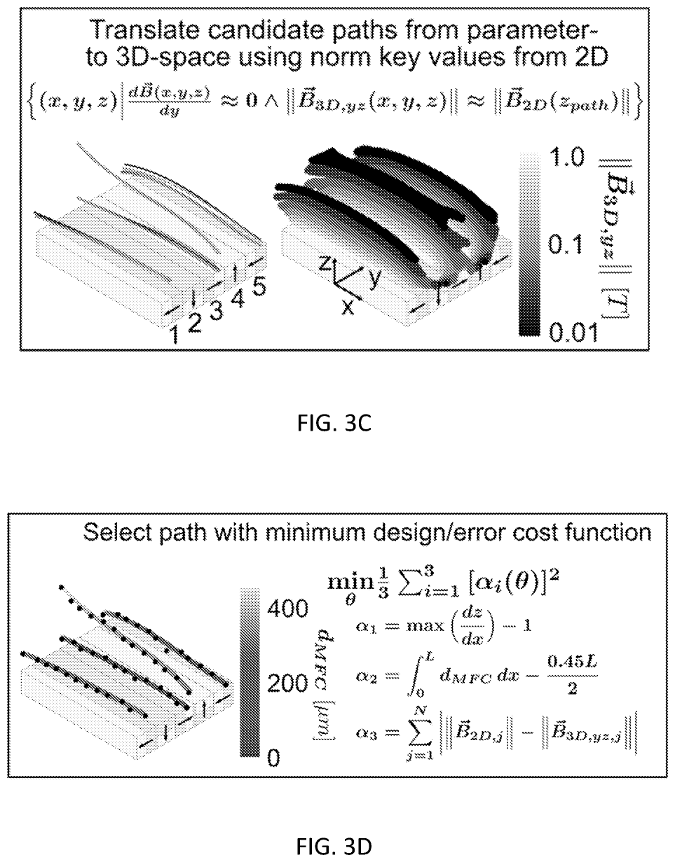

FIG. 3 shows our process to identify the spatial coordinates (x, y, z) for the entire length of the tubing and the MFC in order to achieve an exponential magnetophoretic force profile. Our process consisted of four steps.

In step 1, we generated a target mathematical profile of max ∥(∇·B̅)B̅∥ as a function of the path of the tubing s (FIG. 3A). The upper bound of the target value for max ∥(∇·B̅)B̅∥ metric in FIG. 3A was set at 900 T2/m, equal to the highest value of max ∥(∇·B̅)B̅∥ for zₚₐₜₕ=1 mm and d_{MFC}=0 mm. We selected an exponential function ƒ(s)=−1.9 (1−e^{0.04s}) as a test target profile. Other similar functions are also compatible with our design. The path length s spanned the length of 4 passes of the tubing across the long axes of the magnets (152.4 mm), ignoring the portions of the tubing that looped back outside the MSD since it was exposed to weak magnetic flux.

In step 2, we identified different combinations of zₚₐₜₕ and d_{MFC} that could generate an exponential magnetophoretic force profile along the path of the tubing. To do so, we plotted max ∥(∇·B̅)B̅∥ obtained from the parametric sweep (as described in FIG. 2B) on a surface as a function of zₚₐₜₕ and d_{MFC} (FIG. 3B). We then identified candidate paths (q) on the max ∥(∇·B̅)B̅∥ surface that would generate the exponential profile f(s). Each candidate path consisted of approximately N=150 points on the max ∥(∇·B̅)B̅∥ surface. Each point corresponded to one set of zₚₐₜₕ and d_{MFC} values.

In step 3, we translated the candidate parametric paths into physical paths in 3D (FIG. 3C). The values of d_{MFC} were already defined from the candidate parametric paths in step 2, but zₚₐₜₕ had to be translated into physical positions in 3D. We performed a high-resolution numerical simulation of the magnetic field in free space in 3D over the Halbach array, and identified the set of (x, y, z) points in the 3D simulation that satisfied the following two criteria: 1) d\vec{B}(x, y, z)/dy≈0, and 2) ∥\vec{B}_{3D,yz}(x,y,z)∥≈∥\vec{B}_{2D}(zₚₐₜₕ)∥. The first criterion was necessary to ensure the magnetic flux lines acting on the MFC were horizontal (i.e., the only possible y-locations of the tubing were above magnets 1, 3, or 5). The second criterion matched the norm of the magnetic flux in the yz-plane ∥\vec{B}_{3D,yz}(x, y, z)∥ from the 3D simulation to the norm of the magnetic flux ∥\vec{B}_{2D}(zₚₐₜₕ)∥ from the 2D simulation in FIG. 2. Satisfying these two criteria allowed the matching of the 3D flux concentrated by the MFC to what was simulated in 2D, and thus the value of max ∥(∇·B̅)B̅∥ in the tubing cross-section. The output of step 3 was six sets of 3D candidate paths translated from parameter space that recapitulated our target magnetophoretic force profile. We had limited our number of potential paths to six, but many potential routes through parameter space, and thus through physical space, could be conceived.

In step 4, the six sets of 3D candidate paths were evaluated using a cost function (FIG. 3D) to select for the path that minimized the following functions:

$${α}_{1} = max ( \frac{dz}{dx} ) - 1 , \qquad 1 )$$

which selects for paths with gradual slopes for the tubing to follow in the xz-plane. This function avoids paths with sharp bends which could lead to difficulties in threading the MFC wires through the 3D printed part.

$${α}_{2} = {\int }_{0}^{L} {d}_{MFC} dx - \frac{0.45 L}{2} , \qquad ( 2 )$$

which selects for MFC paths with gradually varying d_{MFC} from 450 to 0 μm across the total length L of the path to facilitate the 3D printing and the threading of the MFC. 3) Σⱼ₌₁^{N}|∥\vec{B}_{2D,j}∥−∥\vec{B}_{3D,yz,j}∥|, which selects for paths that can best match ∥\vec{B}_{3D,yz}(x, y, z)∥ with ∥\vec{B}_{2D}(zₚₐₜₕ)∥ across all N points in the path. The path e with the minimum cost function

$${min}_{θ} \frac{1}{3} {\sum }_{i = 1}^{3} {[ {α}_{i} ( θ ) ]}^{2}$$

was selected as the final position of the tubing and the MFC to construct the MSD.
3.3 Standalone MSD and the i-BID Achieve High Purity and Recovery of Basophil Isolation

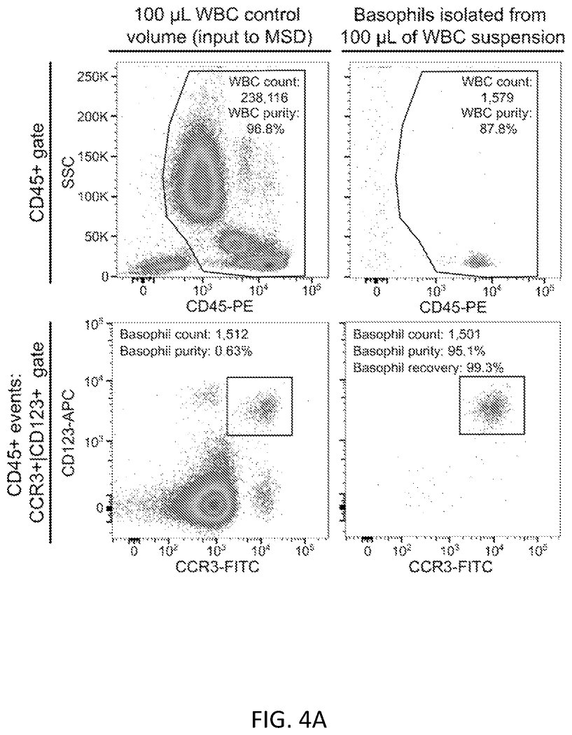

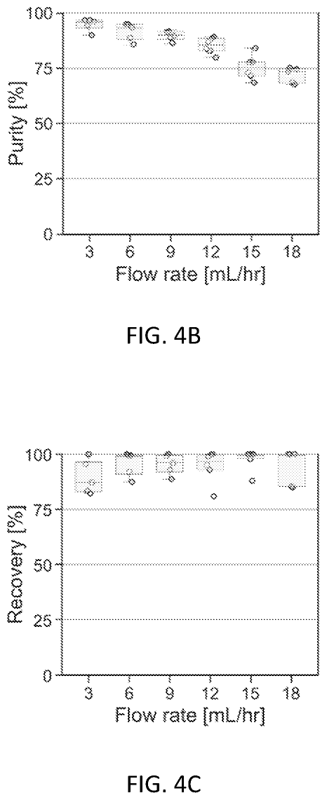

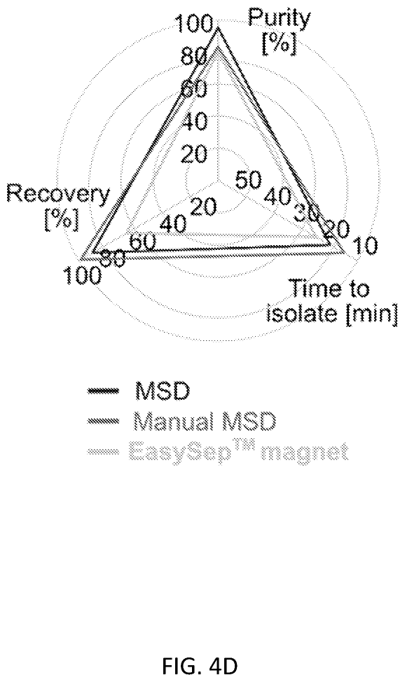

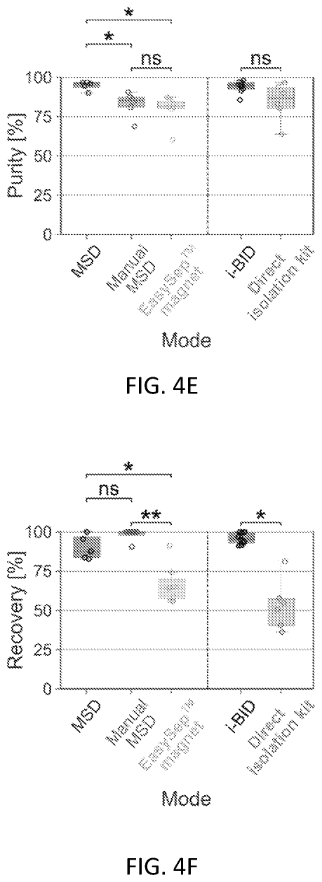

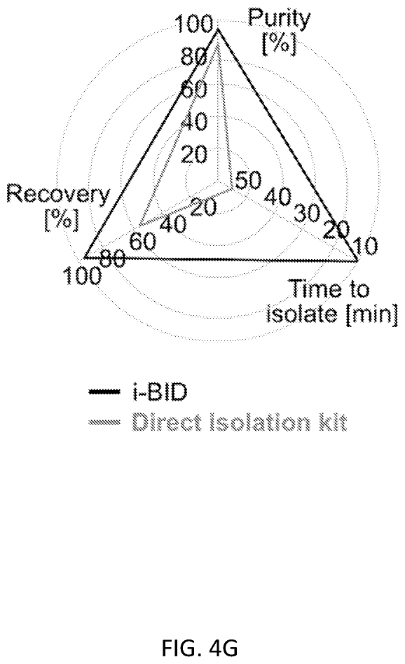

To identify the range of flow rates for effective basophil isolation, we first characterized the standalone MSD using enriched WBCs tagged with MNP off-chip. FIG. 4A shows a representative dot plot of our flow cytometry data. FIG. 4B shows that the purity of isolated basophils was above ˜70% for all flow rates tested, with a maximum mean purity of 94.8% (range 90.0-96.8%, n=5) at 3 mL/hr. From 3 to 12 mL/hr, the purity decreased only gently and remained above 80%. The decrease in purity at high flow rates was expected, likely due to the increased inertial forces from the convective flow overcoming magnetophoretic capture forces.

FIG. 4C shows that the recovery of basophils was above ˜80% for all flow rates tested, with all flow rates exhibiting at least one instance of 100% recovery. Across all flow rates, the mean recovery was 94.6% (range 81.0-100%, n=5). Slightly lower median recovery was observed at 3 mL/hr. This lower recovery was attributed to non-specific capture of basophils caused by magnetic debris build-up on the tubing walls which was exacerbated when the flow was slow with low inertial forces.

Given the high purity and recovery of our standalone MSD and the relative insensitivity to the injection flow rates, we went on to test if we could perform basophil isolation without using any pumps by manually injecting the enriched WBC-MNP/NSAb suspensions through the MSD. This operation allows for a direct comparison of the performance of our MSD with the commercial EasySep magnetic column in bulk MACS assays and could enable the broader use of our MSD in labs that do not have ready accessibility to syringe pumps and microfluidics. We instructed the participants to aim for a steady withdrawal of 100 mL of WBC-MNP/NSAb suspension through the MSD in 2-3 min, corresponding to an average flow rate of 6-8 mL/hr. Using this manual operation of the MSD, we achieved 85.7% purity (range 80.9-90.5%, n=4) and 100.0% recovery (range 106.6-131.7%, n=4) across all participants. Using the EasySep magnetic column, we achieved a purity of 79.2% (range 59.8-87.1%, n=7) and a recovery of 66.9% (range 54.7-90.7%, n=7) (FIG. 4D). These results show the manual MSD had better average purity and markedly higher recovery than EasySep. Importantly, the range of purity and recovery achieved by the manual MSD was much narrower than the magnetic column. The manual MSD thus allows a more consistent and predictable performance than the magnetic column.

Finally, we tested the performance of the fully integrated i-BID. Compared with the standalone MSD, the integrated i-BID allowed for precise control over the incubation time given its integrated mixing and flow-through design. Although the EasySep protocol suggested an incubation time of 5 min, we incubated for 3 min instead because it gave a basophil purity of >90%, which already exceeded the purity of bulk basophil isolation using EasySep (FIG. 4D). We chose to operate the i-BID at flow rates of 3-5 mL/hr due to because the highest purity was obtained at these flow rates in the standalone MSD, and the probability of clogging at the DLD inlet increased at increasing flow rates. FIG. 4D,E show that the mean purity of the BID was 93.5% (range 85.5-97.0%, n=6), and the mean recovery was 96.8% (range 91.3-100.0%, n=6). These results represent >13% improvement in purity and >29% improvement in recovery compared with the magnetic column.

In our i-BID, the entire process from loading whole blood to obtaining isolated basophils was completed in 8-10 min, which was >4.5-fold shorter than bulk MACS assay which took ˜45 min due to multiple centrifugation steps required to remove RBCs. Furthermore, sedimentation methods for removing RBCs and enriching WBCs, commonly used in bulk MACS assays, inherently led to ˜50% loss of basophils in RBC-rich sediment. These sedimentation methods also limit what can reasonably be used as a starting blood volume because of the difficulty in extracting low-volume, WBC-rich buffy coats after sedimentation. The integration of DLD in the i-BID workflow thus enables basophil isolation from small blood volumes with high levels of recovery.

The i-BID does not Alter the Activation Status of Basophils

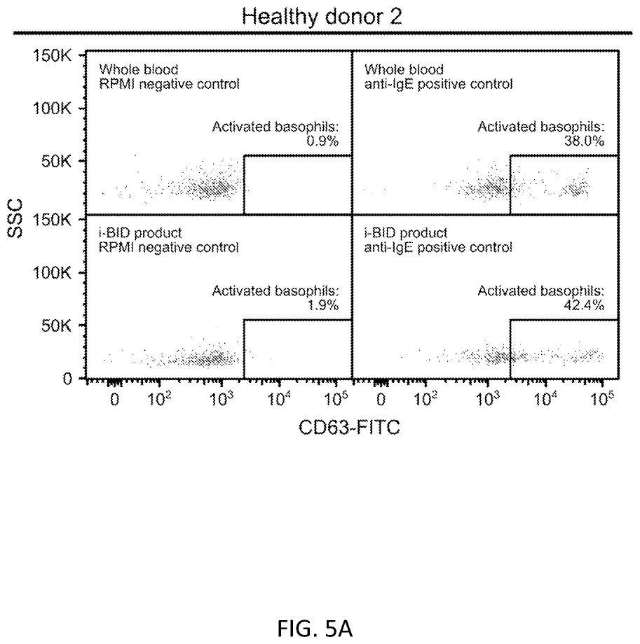

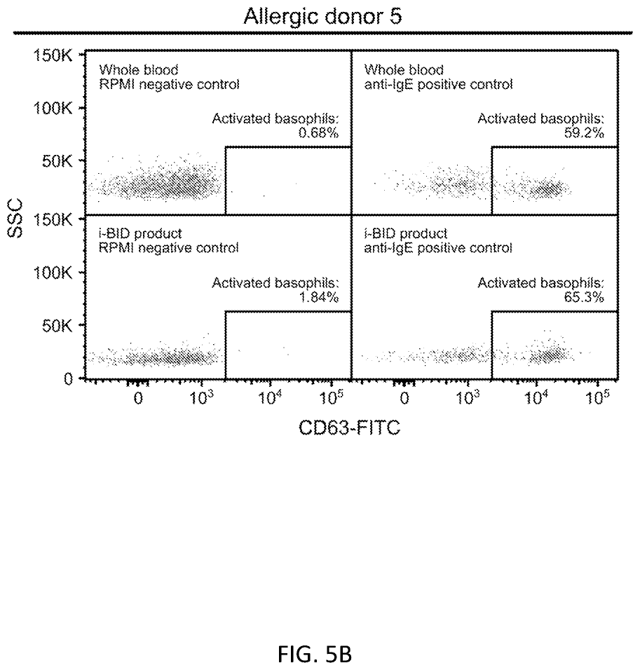

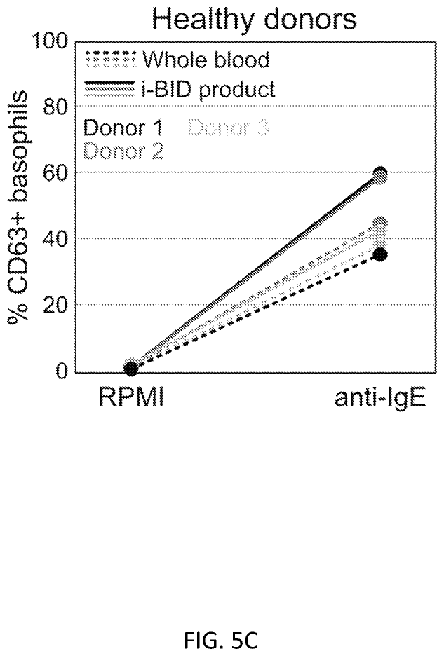

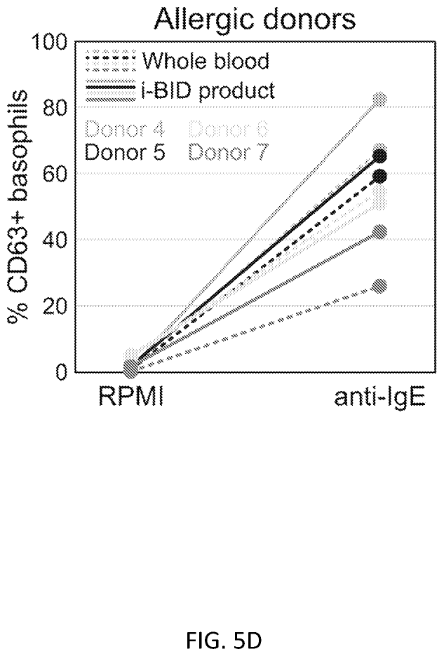

For our method to be useful for downstream basophil assays, it is critical that the i-BID does not induce any unwanted activation of basophils and that they can still be activated by known stimulants (e.g., anti-IgE). FIG. 5 compares the activation status of basophils isolated from our i-BID vs. basophils from whole blood, incubated with RPMI and anti-IgE, respectively. For the whole blood sample, we followed standard protocol for measuring basophil activation (see Methods for details).

FIG. 5A shows that basophils isolated with the i-BID did not exhibit any significant level of activation when incubated with RPMI. Compared with whole blood, the slight increase in the percent of activated basophils in the i-BID sample was likely due to the fact that there were >100 times more cells in the whole blood sample (containing all RBCs and WBCs) than in the i-BID sample (containing only basophils) when the activation stains were added. Therefore, the amount of stain per cell was much higher in the i-BID sample than in the whole blood sample. Despite this slight increase in baseline activation, the percent of activated basophils in the i-BID sample was less than 5%, well below the typical level of baseline activation published in the literature. 63.64 FIG. 5B shows that basophils retained their ability to become activated in response to anti-IgE. Basophils isolated from the i-BID displayed strong activation signals (>40% activated basophils), similar in magnitude to basophils from whole blood (38%).

### BIBLIOGRAPHY

1. Galli, S. J., Gordon, J. R. & Wershil, B. K. Cytokine production by mast cells and basophils. Curr. Opin. Immunol. 3, 865-872 (1991).
2. Karasuyama, H., Miyake, K., Yoshikawa, S. & Yamanishi, Y. Multifaceted roles of basophils in health and disease. J. Allergy Clin. Immunol. 142, 370-380 (2018).
3. Steiner, M., Huber, S., Harrer, A. & Himly, M. The Evolution of Human Basophil Biology from Neglect towards Understanding of Their Immune Functions. Biomed Res. Int. 2016, 8232830 (2016).
4. Miyake, K. & Karasuyama, H. Emerging roles of basophils in allergic inflammation. Allergol Int 66, 382-391 (2017).
5. Chirumbolo, S. State-of-the-art review about basophil research in immunology and allergy: is the time right to treat these cells with the respect they deserve? Blood Transfus. 10, 148-164 (2012).
6. Miyake, K., Shibata, S., Yoshikawa, S. & Karasuyama, H. Basophils and their effector molecules in allergic disorders. Allergy (2020). doi:10.1111/all.14662
7. Borriello, F., Iannone, R. & Marone, G. Histamine Release from Mast Cells and Basophils. Handb Exp Pharmacol 241, 121-139 (2017).
8. Yamanishi, Y. et al. Skin-infiltrating basophils promote atopic dermatitis-like inflammation via IL-4 production in mice. Allergy 75, 2613-2622 (2020).
9. Siracusa, M. C., Kim, B. S., Spergel, J. M. & Artis, D. Basophils and allergic inflammation. J. Allergy Clin. Immunol. 132, 789-801; quiz 788 (2013).
10. Korošec, P., Gibbs, B. F., Rijavec, M., Custovic, A. & Turner, P. J. Important and specific role for basophils in acute allergic reactions. Clin. Exp. Allergy 48, 502-512 (2018).
11. Lin, Y.-H. et al. Selective suppression of oral allergen-induced anaphylaxis by Allergin-1 on basophils in mice. Int. Immunol. 32, 213-219 (2020).
12. Korosec, P. et al. Basophils, high-affinity IgE receptors, and CCL2 in human anaphylaxis. J. Allergy Clin. Immunol. 140, 750-758.e15 (2017).
13. Yamaga, S. et al. Decreased intracellular histamine concentration and basophil activation in anaphylaxis. Allergol Int 69, 78-83 (2020).
14. Piliponsky, A. M. et al. Basophil-derived tumor necrosis factor can enhance survival in a sepsis model in mice. Nat. Immunol. 20, 129-140 (2019).
15. Marone, G. et al. Is there a role for basophils in cancer? Front. Immunol. 11, 2103 (2020).
16. Rodriguez, L. et al. Systems-Level Immunomonitoring from Acute to Recovery Phase of Severe COVID-19. Cell Rep. Med. 1, 100078 (2020).
17. Cohen, M. et al. Lung Single-Cell Signaling Interaction Map Reveals Basophil Role in Macrophage Imprinting. Cell 175, 1031-1044.e18 (2018).
18. Yamanishi, Y., Miyake, K., Iki, M., Tsutsui, H. & Karasuyama, H. Recent advances in understanding basophil-mediated Th2 immune responses. Immunol. Rev. 278, 237-245 (2017).
19. Sokol, C. L. et al. Basophils function as antigen-presenting cells for an allergen-induced T helper type 2 response. Nat. Immunol. 10, 713-720 (2009).
20. Nakashima, C. et al. Basophils regulate the recruitment of eosinophils in a murine model of irritant contact dermatitis. J. Allergy Clin. Immunol. 134, 100-107 (2014).
21. Cheng, L. E. et al. IgE-activated basophils regulate eosinophil tissue entry by modulating endothelial function. J. Exp. Med. 212, 513-524 (2015).
22. Duan, L. et al. Basophil activation test shows high accuracy in the diagnosis of peanut and tree nut allergy: The Markers of Nut Allergy Study. Allergy (2020).
23. Hoffmann, H. J. et al. The clinical utility of basophil activation testing in diagnosis and monitoring of allergic disease. Allergy 70, 1393-1405 (2015).
24. Hemmings, O., Kwok, M., Mckendry, R. & Santos, A. F. Basophil activation test: old and new applications in allergy. Curr. Allergy Asthma Rep. 18, 77 (2018).
25. Özdemir, S. K. et al. Reliability of basophil activation test using CD203c expression in diagnosis of pollen allergy. Am J Rhinol Allergy 25, e225-31 (2011).
26. Steiner, M., Harrer, A. & Himly, M. Basophil Reactivity as Biomarker in Immediate Drug Hypersensitivity Reactions-Potential and Limitations. Front. Pharmacol. 7, 171 (2016).
27. Lotzsch, B., Dölle, S., Vieths, S. & Worm, M. Exploratory analysis of CD63 and CD203c expression in basophils from hazelnut sensitized and allergic individuals. Clin. Transl. Allergy 6, 45 (2016).
28. MacGlashan, D. Expression of CD203c and CD63 in human basophils: relationship to differential regulation of piecemeal and anaphylactic degranulation processes. Clin. Exp. Allergy 40, 1365-1377 (2010).
29. Mukai, K. et al. Assessing basophil activation by using flow cytometry and mass cytometry in blood stored 24 hours before analysis. J. Allergy Clin. Immunol. 139, 889-899.e11 (2017).
30. Hamey, F. K. et al. Single-cell molecular profiling provides a high-resolution map of basophil and mast cell development. Allergy 76, 1731-1742 (2021).
31. Sasaki, H., Kurotaki, D. & Tamura, T. Regulation of basophil and mast cell development by transcription factors. Allergol Int 65, 127-134 (2016).
32. Winter, N. A. et al. Sputum mast cell/basophil gene expression relates to inflammatory and clinical features of severe asthma. J. Allergy Clin. Immunol. (2021).
33. Chhiba, K. D., Hsu, C.-L., Berdnikovs, S. & Bryce, P. J. Transcriptional Heterogeneity of Mast Cells and Basophils upon Activation. J. Immunol. 198, 4868-4878 (2017).
34. Hwang, B., Lee, J. H. & Bang, D. Single-cell RNA sequencing technologies and bioinformatics pipelines. Exp Mol Med 50, 1-14 (2018).
35. Amamoto, R. et al. Probe-Seq enables transcriptional profiling of specific cell types from heterogeneous tissue by RNA-based isolation. Elife 8, (2019).
36. Nguyen, Q. H., Pervolarakis, N., Nee, K. & Kessenbrock, K. Experimental Considerations for Single-Cell RNA Sequencing Approaches. Front. Cell Dev. Biol. 6, 108 (2018).
37. Shiono, H., Matsui, T., Okada, T. & Ito, Y. Single-step enrichment of basophils from human peripheral blood by a novel method using a Percoll density gradient. J Sep Sci 39, 3062-3071 (2016).
38. Degenhart, H. J., van der Maarel, J. W., Jongkind, H., Raatgeep, H. C. & Neijens, H. J. Properties of human basophils isolated by fluorescence-activated cell sorting. J. Allergy Clin. Immunol. 82, 455-461 (1988).
39. Gibbs, B. F. & Falcone, F. H. Basophils and mast cells: methods and protocols. 2163, 35-48 (Springer US, 2020).
40. EasySep™ Human Basophil Isolation Kit|STEMCELL Technologies. at (stemcell.com/easysep-human-basophil-isolation-kit.html)
41. Gibbs, B. F., Papenfuss, K. & Falcone, F. H. A rapid two-step procedure for the purification of human peripheral blood basophils to near homogeneity. Clin. Exp. Allergy 38, 480-485 (2008).
42. MacGlashan, D. Expression profiling of human basophils: modulation by cytokines and secretagogues. PLOS One 10, e0126435 (2015).
43. Tsang, S., Hayashi, M., Zheng, X., Campbell, A. & Schellenberg, R. R. Simplified purification of human basophils. J. Immunol. Methods 233, 13-20 (2000).
44. Hou, H. W. et al. Microfluidic devices for blood fractionation. Micromachines (Basel) 2, 319-343 (2011).
45. Pratt, E. D., Huang, C., Hawkins, B. G., Gleghorn, J. P. & Kirby, B. J. Rare Cell Capture in Microfluidic Devices. Chem Eng Sci 66, 1508-1522 (2011).
46. Cheng, X. et al. A microfluidic device for practical label-free CD4 (+) T cell counting of HIV-infected subjects. Lab Chip 7, 170-178 (2007).
47. Lang, J. M., Casavant, B. P. & Beebe, D. J. Circulating tumor cells: getting more from less. Sci. Transl. Med. 4, 141ps13 (2012).
48. Nagrath, S. et al. Isolation of rare circulating tumour cells in cancer patients by microchip technology. Nature 450, 1235-1239 (2007).
49. Pezzi, H. M. et al. Versatile exclusion-based sample preparation platform for integrated rare cell isolation and analyte extraction. Lab Chip 18, 3446-3458 (2018).
50. Augustsson, P., Karlsen, J. T., Su, H.-W., Bruus, H. & Voldman, J. Iso-acoustic focusing of cells for size-insensitive acousto-mechanical phenotyping. Nat. Commun. 7, 11556 (2016).
51. Sethu, P., Sin, A. & Toner, M. Microfluidic diffusive filter for apheresis (leukapheresis). Lab Chip 6, 83-89 (2006).
52. Hejazian, M., Li, W. & Nguyen, N.-T. Lab on a chip for continuous-flow magnetic cell separation. Lab Chip 15, 959-970 (2015).
53. Pamme, N. Magnetism and microfluidics. Lab Chip 6, 24-38 (2006).
54. Mishra, A. et al. Ultrahigh-throughput magnetic sorting of large blood volumes for epitope-agnostic isolation of circulating tumor cells. Proc. Natl. Acad. Sci. USA 117, 16839-16847 (2020).
55. Sun, C. et al. Paramagnetic Structures within a Microfluidic Channel for Enhanced Immunomagnetic Isolation and Surface Patterning of Cells. Sci. Rep. 6, 29407 (2016).
56. D'Silva, J., Austin, R. H. & Sturm, J. C. Inhibition of clot formation in deterministic lateral displacement arrays for processing large volumes of blood for rare cell capture. Lab Chip 15, 2240-2247 (2015).
57. Feng, S. L., Skelley, A. M., Anwer, A. G., Liu, G. & Inglis, D. W. Maximizing particle concentration in deterministic lateral displacement arrays. Biomicrofluidics 11, 024121 (2017).
58. McGrath, J., Jimenez, M. & Bridle, H. Deterministic lateral displacement for particle separation: a review. Lab Chip 14, 4139-4158 (2014).
59. Davis, J. A. Microfluidic Separation of Blood Components through Deterministic Lateral Displacement′ (Princeton University, 2008) at (scholar.princeton.edu/sites/default/files/sturm/files/davis-thesis.pdf)
60. Boxshall, K. et al. Simple surface treatments to modify protein adsorption and cell attachment properties within a poly(dimethylsiloxane) micro-bioreactor. Surf. Interface Anal. 38, 198-201 (2006).
61. Rhoades, T., Kothapalli, C. R. & Fodor, P. S. Mixing optimization in grooved serpentine microchannels. Micromachines (Basel) 11, (2020).
62. Procedure: Human Basophil Enrichment Kit. at (cdn.stemcell.com/media/files/pis/29109-PIS_1_0_0.pdf)
63. Sturm, E. M. et al. CD203c-based basophil activation test in allergy diagnosis: characteristics and differences to CD63 upregulation. Cytometry B Clin Cytom 78, 308-318 (2010).
64. Mukai, K. et al. A new fluorescent-avidin-based method for quantifying basophil activation in whole blood. J. Allergy Clin. Immunol. 140, 1202-1206.e3 (2017).

### Example 2

Alternative Means for Enrichment of White Blood Cells from Whole Blood

Alternative methods of on-chip WBC enrichment from whole blood, apart from deterministic lateral displacement may be employed. An example of one such method involves the use of secondary Dean flow velocity fields that form when fluid passes through a curved microchannel. Due to size differences, WBCs and RBCs assume different lateral equilibrium positions in these secondary flow vortices, which enables separation of WBCs from RBCs at the channel outlet.

Another method for WBC enrichment from whole blood involves applying a hydrophoretic force generated from periodic pinching and expansion of the fluid flow by periodically varying microchannel width or by perpendicular or angled steps on the channel floor. The generated focusing flows deflect cells differently depending on mass and volume, allowing the cells to be sorted into specific outlets based on their size. One example is using slanted channels which alternate position on the floor and ceiling of the channel. Another example involves angled steps which do not run the entire width of the channel. d

Blood cell types may also be separated using active methods. For example, acoustic radiation force may be used to separate cells based on size, density and compressibility. Alternatively, cells may be sorted based on their equilibrium position under a non-uniform electric field. Cell types are separated based on differing dielectrophoretic activity in the electric field.

RBCs may also be separated from WBCs using non-microfluidic methods. One example is through the use of bulk separation, such as density gradient centrifugation.

Methods for WBC enrichment may involve the addition of cell media, e.g., 1×PBS, to dilute whole blood or the removal of media following WBC separation to concentrate cells for downstream magnetic isolation of target cells.

Separation methods that favor high recovery of WBCs and low incidence of microchannel fouling/clogging may be employed upstream of the magnetic separation processes. DLD inlet channels leading to the pillar array can be designed such that a gradual increase in shear stress is applied to lessen the effect of shear induced activation of platelets and WBCs that is caused by a sudden increase in shear at the onset of the pillar array.

### Example 3

### Control of Flow Rate and Pressure

A plurality of pumps and sensors are used to control the flow rates and pressures in the magnetophoretic separation device. These pumps may be a combination of positive displacement pumps or pressure generating pumps. A combination of pumps is used, which may include syringe pumps, diaphragm pumps, peristaltic pumps or piston pumps, or a combination thereof, operating under control feedback from sensors in the system that monitor flow rate and/or pressure. Blood, buffer, reagents, and waste can be injected or withdrawn simultaneously across single or parallelized channels of separate isolations using any combination of pumping methods. The system is capable of maintaining target flow rates and pressures that are required to operate all microfluidic processes (i.e., WBC enrichment and immunomagnetic negative selection of basophils) of the isolation system in a steady-state manner. Flow rates through the magnetic separation device may range from 0.01 mL/hr-2000 mL/hr. Whole blood and buffer injection flow rates into the WBC enrichment stage will be set based on the target MSD flow rate and the geometry of the WBC and RBC outlets of the WBC enrichment channels, e.g., DLD channels.

### Example 4

### Geometry of Channels in the MSD

The geometry of the channels in the MSD is set to position the fluid flow and cell path in the vicinity of the magnetic force field and to provide space for captured magnetically-tagged cells to accumulate. The number of captured cells can be adjusted by changing the position of the channel relative to the magnets and flux concentrator (if present), or by changing the fluid velocity in the channel. The fluid velocity in the MSD can be adjusted by changing the input flow rate, by changing the cross-sectional area of the channel, or by a diversion of some fluid to waste. Channel geometry may be planar (e.g., embodiment represented in FIG. 11 and FIG. 13), multi-layered, or 3-dimensional (e.g., embodiment represented in FIG. 9). Some embodiments have a subset of straight fluid paths (e.g., embodiment represented in FIG. 13), curved fluid paths and repeating serpentine elements (e.g., embodiment represented in FIG. 11). In certain embodiments, the fluid path can be made up of tubing. In certain embodiments, the fluid path can be machined, embossed, rastered, or etched into solid material. In certain embodiments, the fluidic channel can be made through lithography, additive manufacturing, casting or forging. MSD channel width and height can vary from 0.01-100 mm. MSD channel length can vary from 1-1000 mm. The MSD cross sectional shape may be square, rectangular, circular, elliptical, or polygonal.

Fluidic elements in the channels may focus all or a subset of cells towards an area of higher magnetic force field. This may be done using constrictions, expansions, vanes or sheath flow. This flow focusing of cells may improve the outlet purity or recovery percentage of a target cell type exiting the magnetic separation device.

Some versions may use fluidic elements to mix the fluid either before the MSD, or periodically within the MSD. One function of this mixing is to rearrange the cell streamlines in the channel to ensure all magnetically tagged cells experience a sufficiently large magnetic force field to be captured. This mixing may be done in a variety of ways including serpentine channels, staggered herringbones, dean vortices, and parallel lamination.

Certain embodiments of the MSD may include fluidic features which act as gutters where captured magnetically tagged cells and magnetic nanoparticles can accumulate. These gutters can prevent clogging of the entire fluid path by preventing accumulation from filling the full fluidic cross section and blocking the flow path of target cells. Some embodiments can combine serpentine channels and gutters to place the intended accumulation area near to the region of the highest magnetic force (e.g., FIG. 11). Another type of embodiment bifurcates fluid flow in regions of concentrated magnetic flux to direct cells closer to high magnetic gradients.

### Example 5

### Design of Magnetic Force Field in Vicinity of Fluidic Channels

Magnetic flux can be generated by permanent magnets or by arrangements of electromagnets. Target magnetic flux density can range from 0.1-10 T and can be applied to capture magnetically tagged non-target cells from a flowing suspension in conjunction with magnetic flux concentrators (MFCs) or on their own. There is a plurality of configurations in which permanent magnets can be arranged to increase the overall magnetic flux in the vicinity of the fluidic channels. These configurations include, but not limited to: forms of Halbach arrays, multiple magnets with opposing polarities, multiple magnets with aligned polarities, grid/array of magnets with alternating polarities, and self-assembling magnetic colloids.

MFCs serve to redirect and concentrate the magnetic field to be sufficiently close to fluidic channels that tagged cells can be captured. They can be fabricated from various magnetic materials with relative magnetic permeability ranging from 100-100,000 and magnetic saturation point ranging from 0.1-3.0 T. MFCs can be fabricated from 10-20,000 μm sheets of metal alloy composed of iron, nickel, and cobalt. MFCs materials can come in the form of sheets, wires, extrusions, CNC machined parts, or metal 3-D printed parts. MFCs can be shaped to direct magnetic flux density into the channel. The radii of MFCs can be adjusted to change the magnitude of grad(B). This can be used to tune the peak magnitude of the magnetic force on cells in the MSD channels, as well as the decay of the magnetic force field away from the MFC. Some embodiments of MFCs are comprised of fluidic channels filled with ferrofluids or self-assembled magnetic colloids.

### Example 6

### Parallelization of the MSD

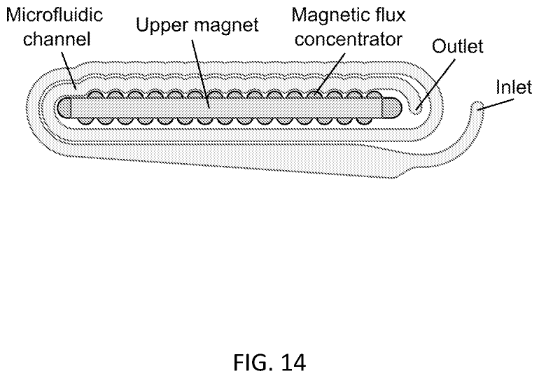

Some embodiments of the MSD may feature multiple channels (i.e., more than 2 channels) in parallel, with each channel independently separating target cells from a different sample of blood. A schematic representation of one embodiment of a parallelized MSD is shown in FIG. 13. In some embodiments, parallel channels can be pumped using a shared pumping system using common pressure nodes across parallel channels or using a positive displacement pump at the inlet or outlet. Other embodiments may use separate pumping systems for each individual channel. Flow rate and pressure sensors for control feedback can be applied to each parallel channel or to select channels to measure representative flow rates and pressures that are expected in all parallel channels. The outlets of the parallel channels may remain separate or they may flow into a common collection reservoir. In some versions, the flow rates in the parallel channels may be identical. In other versions, the flow rates in the parallel channels may differ. In some versions, parallel channels can share magnet arrays and magnetic flux concentrators with other channels. In other embodiments, each parallel channel has its own magnet array and/or MFC. In some embodiments, the fluid path may make multiple passes adjacent to the same magnet, magnet array or magnetic flux concentrator. The multiple passes can be in the form of the fluid path wrapping repeatedly around a magnet array or magnetic flux concentrator, by serpentines in the fluid path running adjacent to the magnet array or magnetic flux concentrator, or any other geometric configuration. FIG. 14 shows a schematic representation of an MSD embodiment that wraps the magnet and a magnetic flux concentrator for multiple passes through the magnetic field.

What is claimed is:

1. A magnetophoretic separation device (MSD) comprising:
a) a single magnet or a magnetic array comprising a plurality of magnets;
b) a scaffold comprising a slot to secure the single magnet or the magnetic array and one or more channels, wherein each channel comprises one or more fluidic conduits connected to one or more outlets; and
c) one or more magnetic flux concentrators in proximity to the one or more fluidic conduits, wherein spacing of the one or more magnetic flux concentrators relative to the single magnet or the magnetic array and the one or more fluidic conduits is selected to produce a target magnetophoretic gradient profile, wherein magnetophoretic force varies along the length of the one or more fluidic conduits.

2. The MSD of claim 1, wherein the magnetic array is a Halbach array.

3. The MSD of claim 2, wherein the Halbach array is a cylindrical Halbach array comprising a plurality of ring magnets or a linear Halbach array comprising a plurality of bar magnets.

4. The MSD of claim 1, wherein the magnetic array comprises multiple magnets with opposing polarities, multiple magnets with aligned polarities, a grid of magnets with alternating polarities, or self-assembling magnetic colloids.

5. The MSD of any one of claims 1-4, wherein the magnetic array comprises at least five magnets.

6. The MSD of any one of claims 1-5, wherein the magnets are permanent magnets or electromagnets.

7. The MSD of claim 6, wherein the permanent magnets are neodymium magnets.

8. The MSD of any one of claims 1-7, wherein the one or more fluidic conduits are provided by fluidic tubing positioned adjacent to the magnetic array or the magnetic flux concentrator.

9. The MSD of claim 8, wherein the fluidic tubing has an inner diameter sufficiently small that magnetic flux from the one or more magnetic flux concentrators reaches the center axis of the fluidic tubing.

10. The MSD of claim 9, wherein the inner diameter is sufficiently large to retain captured non-target cells without obstructing the one or more types of target cells from flowing through the fluidic tubing.

11. The MSD of any one of claims 1-7, wherein the one or more fluidic conduits are machined, embossed, rastered, or etched into the one or more channels.

12. The MSD of any one of claims 1-11, wherein the one or more channels are planar, multi-layered, or three-dimensional.

13. The MSD of any one of claims 1-12, wherein each fluidic conduit provides a straight fluid path, a curved fluid path, or a serpentine fluid path.

14. The MSD of claim 13, wherein the fluid path revolves around the plurality of ring magnets.

15. The MSD of any one of claim 13, wherein the fluidic path runs parallel to the plurality of bar magnets.

16. The MSD of any one of claims 1-15, wherein the fluidic path is bifurcated to direct fluid flow to regions of concentrated magnetic flux.

17. The MSD of any one of claims 1-16, wherein the channel width ranges from 0.01 mm to 100 mm.

18. The MSD of any one of claims 1-17, wherein the channel height ranges from 0.01 mm to 100 mm.

19. The MSD of any one of claims 1-18, wherein the channel length ranges from 1 mm to 1000 mm.

20. The MSD of any one of claims 1-19, wherein the one or more fluidic conduits have a square, rectangular, circular, elliptical, or polygonal cross-sectional shape.

21. The MSD of any one of claims 1-20, wherein the one or more channels further comprise one or more fluidic elements that focus all or a subset of cells from a fluid sample towards an area of higher magnetic force field.

22. The MSD of claim 21, wherein one or more fluidic elements comprise a constriction, an expansion, a vane, or a sheath flow device, or a combination thereof.

23. The MSD of any one of claims 1-22, further comprising one or more gutters connected to the one or more fluidic conduits.

24. The MSD of any one of claims 1-23, wherein the one or more fluidic conduits further comprise a staggered herringbone mixer, dean vortices, parallel lamination of fluid streamlines, micropillars, or steps.

25. The MSD of any one of claims 1-24, wherein the magnetic flux concentrator produces a magnetic flux density ranging from 0.1 tesla to 10 tesla.

26. The MSD of any one of claims 1-25, wherein the magnetic flux concentrator has a relative magnetic permeability ranging from 100 to 100,000.

27. The MSD of any one of claims 1-26, wherein the magnetic flux concentrator comprises a fluidic channel comprising a ferrofluid or a self-assembled magnetic colloid.

28. The MSD of any one of claims 1-27, wherein the magnetic flux concentrator radius can be adjusted to tune peak magnitude of the magnetic force on cells in the one or more fluidic conduits.

29. The MSD of any one of claims 1-28, wherein the magnetophoretic force increases exponentially along the length of the one or more fluidic conduits.

30. The MSD of any one of claims 1-29, wherein the MSD comprises two or more channels, each channel comprising a separate fluidic conduit connected to a different outlet, wherein each channel can be used to separate a different type of target cell from non-target cells from a different fluid sample.

31. The MSD of claim 30, wherein the two or more channels are parallel to each other.

32. The MSD of claim 31, wherein the MSD comprises two or more magnetic flux concentrators, wherein the magnetic flux concentrators are parallel to the two or more channels.

33. The MSD of any one of claims 30-32, wherein each outlet is connected to a separate collection reservoir or a common collection reservoir.

34. The MSD of any one of claims 30-33, wherein each channel or a subset of channels shares the same magnetic array and magnetic flux concentrator, or each channel has a different magnetic array and magnetic flux concentrator.

35. The MSD of any one of claims 1-34, further comprising a plurality of pumps.

36. The MSD of claim 35, wherein the pumps are syringe pumps, diaphragm pumps, peristaltic pumps or piston pumps, or any combination thereof.

37. The MSD of claim 35, wherein the pumps are positive displacement pumps or pressure generating pumps.

38. The MSD of any one of claims 1-37, further comprising a plurality of sensors that monitor flow rate or pressure, or a combination thereof.

39. The MSD of claim 38, wherein the pumps and sensors are configured to maintain the flow rate through the magnetic separation device in a range from 0.01 mL/hr to 20,000 L/hr.

40. The MSD of any one of claims 1-39, wherein the one or more fluidic conduits share magnetic arrays, magnetic flux concentrators, pumps, mixing elements, or flow through cytometers, or any combination thereof.

41. The MSD of any one of claims 1-40, wherein each magnetic flux concentrator comprises a plurality of ferromagnetic wires, sheets, self-assembling colloids, or other configuration of ferromagnetic material running alongside one or more of the fluidic conduits, wherein spacing of the wires, sheets, self-assembling colloids, or other configuration of ferromagnetic material relative to the magnetic array and the one or more of the fluidic conduits is selected to produce the target magnetophoretic gradient profile.

42. The MSD of any one of claims 1-41, wherein the MSD is contained in a microfluidic cartridge comprising a first assembly comprising the one or more channels and the one or more magnetic flux concentrators; and a second assembly comprising the magnetic array.

43. A microfluidic magnetic device for isolation of one or more types of target cells from a fluid sample comprising a heterogenous population of cells, the device comprising:
a) one or more inlets to receive the fluid sample;
b) one or more mixers connected to the one or more inlets, wherein the one or more mixers mix magnetic nanoparticles (MNPs) and one or more negative selection binding agents with the cells, wherein the one or more negative selection binding agents selectively bind to one or more surface markers on non-target cells, wherein the one or more surface markers are not present on the one or more types of target cells;
c) one or more incubation tubings connected to the one or more mixers, wherein the cells are incubated with the MNPs and the one or more negative selection binding agents in the one or more incubation tubings, wherein the non-target cells that are bound to the one or more negative selection binding agents are selectively magnetically labeled with the MNPs;
d) the MSD of any one of claims 1-39, wherein the one or more channels of the MSD are connected to the one or more incubation tubings, wherein the MSD captures the magnetically labeled non-target cells and outputs the one or more types of target cells; and
e) one or more outlets connected to the MSD, wherein the one or more types of target cells are expelled from the one or more outlets.

44. The microfluidic magnetic device of claim 43, further comprising one or more deterministic lateral displacement (DLD) channels or curved microchannels that sort cells based on size, wherein each DLD channel or curved microchannel is positioned between one of the inlets and one of the mixers, wherein an input end of each DLD channel or curved microchannel is connected to one of the inlets and an output end of each DLD channel or microchannel is connected to one of the mixers.

45. The microfluidic magnetic device of claim 44, wherein the one or more DLD channels and one or more mixers are contained on a microfluidic chip comprising a first layer comprising the mixers and a second layer comprising the DLD channels.

46. The microfluidic magnetic device of claim 45, wherein the first layer comprising the one or more mixers is above the second layer comprising the one or more DLD channels.

47. The microfluidic magnetic device of any one of claims 43-46, wherein the one or more DLD channels further comprise a coating to reduce cell adhesion to the DLD channels.

48. The microfluidic magnetic device of claim 47, wherein the coating comprises a non-ionic surfactant.

49. The microfluidic magnetic device of any one of claims 43-48, further comprising a means for applying a secondary Dean flow velocity field or a hydrophoretic force to the one or more curved microchannels when fluid passes through the curved microchannels.

50. The microfluidic magnetic device of any one of claims 44-49, wherein the one or more curved microchannels vary in width along the length of the curved microchannels.

51. The microfluidic magnetic device of any one of claims 44-50, wherein the one or more curved microchannels further comprise perpendicular or angled steps.

52. The microfluidic magnetic device of any one of claims 44-51, wherein the one or more types of target cells comprise an immune cell, a blood cell, a stem cell, or a cancer cell, or any combination thereof.

53. The microfluidic magnetic device of claim 52, wherein the immune cell is a basophil, a neutrophil, an eosinophil, a mast cell, a monocyte, a dendritic cell, a macrophage, a T cell, a B cell, or a natural killer cell.

54. The microfluidic magnetic device of claim 53, wherein the basophil is an activated basophil or an unactivated basophil.

55. The microfluidic magnetic device of any one of claims 43-54, wherein the fluid sample is a blood sample.

56. The microfluidic magnetic device of claim 55, wherein the one or more DLD channels or curved microchannels separate white blood cells from red blood cells.

57. The microfluidic magnetic device of any one of claims 43-56, wherein the one or more mixers comprise one or more Herringbone-grooved serpentine channels.

58. The microfluidic magnetic device of claim 57, wherein each Herringbone-grooved serpentine channel has dimensions of about 200 μm in width and about 70 μm in height.

59. The microfluidic magnetic device of claim 57 or 58, wherein each Herringbone-grooved serpentine channel comprises a groove having a height of about 30 μm.

60. The microfluidic magnetic device of any one of claims 43-59, wherein the one or more negative selection binding agents are an antibody, an antibody mimetic, a peptoid, an aptamer, or a ligand.

61. The microfluidic magnetic device of any one of claims 43-60, wherein the microfluidic magnetic device further comprises a flow-through microfluidic cytometer capable of measuring numbers and properties of the one or more types of target cells, wherein the flow-through microfluidic cytometer is fluidically connected to the magnetophoretic separation device.

62. The microfluidic magnetic device of any one of claims 43-61, further comprising a plurality of pumps.

63. The microfluidic magnetic device of claim 62, wherein the pumps are syringe pumps, diaphragm pumps, peristaltic pumps or piston pumps, or any combination thereof.

64. The microfluidic magnetic device of claim 62, wherein the pumps are positive displacement pumps or pressure generating pumps.

65. The microfluidic magnetic device of any one of claims 43-64, further comprising a plurality of sensors that monitor flow rate or pressure, or a combination thereof.

66. The microfluidic magnetic device of claim 65, wherein the pumps and sensors are configured to maintain the flow rate through the magnetic separation device in a range from 0.01 mL/hr to 20,000 L/hr.

67. The microfluidic magnetic device of any one of claims 43-66, where the microfluidic magnetic device comprises a plurality of the MSDs to allow processing of parallel samples.

68. A method of using the microfluidic magnetic device of any one of claims 43-67, the method comprising:
a) introducing the fluid sample comprising the heterogenous population of cells into the one or more inlets;
b) mixing the magnetic nanoparticles (MNPs) and the one or more negative selection binding agents with the cells using the one or more mixers, wherein the one or more negative selection binding agents selectively bind to the one or more surface markers on the non-target cells, wherein the one or more surface markers are not present on the one or more types of target cells;
c) incubating the cells with the MNPs and the one or more negative selection binding agents in the one or more incubation tubings, wherein the non-target cells that are bound to the one or more negative selection binding agents are selectively magnetically labeled with the MNPs;
d) applying a magnetophoretic gradient to capture the magnetically labeled non-target cells from the fluid sample using the MSD; and
e) collecting the one or more types of target cells expelled from the one or more outlets.

69. The method of claim 68, wherein the microfluidic magnetic device comprises one or more deterministic lateral displacement (DLD) channels or a curved microchannels, wherein the method further comprises flowing the fluid sample into the one or more DLD channels or curved microchannels, wherein the one or more DLD channels or curved microchannels separate cells based on size.

70. The method of claim 68 or 69, wherein the fluid sample is a blood sample.

71. The method of claim 70, wherein the one or more DLD channels or curved microchannels separate white blood cells from red blood cells.

72. The method of claim 70, further comprising separating white blood cells from red blood cells by density gradient centrifugation, wherein said introducing the fluid sample into the one or more inlets comprises introducing a fluid sample comprising the separated white blood cells into the one or more inlets.

73. The method of claim 70, further comprising sorting cells by using an acoustic radiation force or dielectrophoretic activity in an electric field prior to said mixing the nanoparticles (MNPs) and the one or more negative selection binding agents with the cells.

74. A microfluidic magnetic device for isolation of one or more types of target cells from a blood sample, the device comprising:
a) one or more inlets to receive a blood sample;
b) one or more deterministic lateral displacement (DLD) channels connected to the inlets, wherein the one or more DLD channels separate white blood cells from red blood cells in the blood sample;
c) one or more mixers connected to the one or more DLD channels, wherein the DLD channels mix magnetic nanoparticles (MNPs) and one or more negative selection binding agents with the separated white blood cells, wherein the one or more negative selection binding agents selectively bind to one or more surface markers on non-target cells, wherein the one or more surface markers are not present on the one or more types of target cells;
d) one or more incubation tubings connected to the one or more mixers, wherein the white blood cells are incubated with the MNPs and the one or more negative selection binding agents in the one or more incubation tubings, wherein the non-target cells that are bound to the one or more negative selection binding agents are selectively magnetically labeled with the MNPs;
e) a magnetophoretic separation device (MSD) connected to the one or more incubation tubings, wherein the MSD applies an exponentially increasing magnetophoretic gradient to capture the magnetically labeled non-target cells; and
f) one or more outlets connected to the MSD, wherein the one or more types of target cells are expelled from the one or more outlets.

75. The microfluidic magnetic device of claim 74, wherein the one or more types of target cells comprise an activated basophil or an unactivated basophil.

76. The microfluidic magnetic device of claim 74 or 75, wherein the one or more negative selection binding agents are antibodies, antibody mimetics, aptamers, peptoids, or ligands.

77. The microfluidic magnetic device of claim 76, wherein the antibodies comprise an anti-HLA-DR antibody, an anti-CD2 antibody, an anti-CD3 antibody, an anti-CD14 antibody, an anti-CD15 antibody, an anti-CD16 antibody, an anti-CD19 antibody, an anti-CD20 antibody, an anti-CD24 antibody, an anti-CD34 antibody, an anti-CD36 antibody, an anti-CD45RA antibody, an anti-CD56 antibody, an anti-CD66b antibody, or an anti-glycophorin A antibody, or any combination thereof.

78. A kit comprising the MSD of any one of claims 1-42 or the microfluidic magnetic device of any one of claims 43-67 and 74-77 and instructions for isolating one or more types of target cells from a fluid sample.

79. The kit of claim 78, further comprising one or more negative selection binding agents that selectively bind to one or more surface markers on non-target cells, wherein the surface marker is not present on the one or more types of target cells.

80. The kit of claim 79, wherein the one or more negative selection binding agents are antibodies, antibody mimetics, aptamers, peptoids, or ligands.

81. The kit of any one of claims 78-80, further comprising magnetic nanoparticles.

82. The kit of any one of claims 78-81, wherein the one or more types of target cells comprise a basophil, a neutrophil, an eosinophil, a mast cell, a monocyte, a dendritic cell, a macrophage, a T cell, a B cell, a natural killer cell, or a stem cell, or any combination thereof.

83. The kit of claim 82, wherein the basophil is an activated basophil or an unactivated basophil.

84. A computer implemented method for controlling the microfluidic magnetic device of any one of claims 43-67, the computer performing steps comprising:
a) injecting the MNPs and the one or more negative selection binding agents into the one or more DLD channels or curved microchannels through the one or more inlets;
b) injecting the fluid sample into the one or more DLD channels or curved microchannels through the one or more inlets;
c) adjusting fluidic flow to a target steady rate;
d) controlling amount of time the cells are incubated with the MNPs and the one or more negative selection binding agents in the one or more incubation tubings; and
e) controlling flow of the cells through the MSD while applying an increasing magnetophoretic gradient to capture the magnetically labeled non-target cells from the fluid sample using the MSD.

85. A non-transitory computer-readable medium comprising program instructions that, when executed by a processor in a computer, causes the processor to perform the method of claim 84.

86. A system for isolating one or more types of target cells from a blood sample, the system comprising:
a) a microfluidic magnetic device for isolation of the one or more target cells from the blood sample, the device comprising:
(i) one or more inlets to receive a blood sample;
(ii) one or more deterministic lateral displacement (DLD) channels or curved microchannels connected to the one or more inlets, wherein the one or more DLD channels or curved microchannels separate white blood cells from red blood cells in the blood sample;
(iii) one or more mixers connected to the one or more DLD channels or curved microchannels, wherein the one or more mixers mix magnetic nanoparticles (MNPs) and one or more negative selection binding agents with the separated white blood cells, wherein the one or more negative selection binding agents selectively bind to one or more surface markers on non-target cells, wherein the one or more surface markers are not present on the one or more types of target cells;
(iv) one or more incubation tubings connected to the one or more mixers, wherein the white blood cells are incubated with the MNPs and the one or more negative selection binding agents in the one or more incubation tubings, wherein the non-target cells that are bound to the one or more negative selection binding agents are selectively magnetically labeled with the MNPs;
(v) a magnetophoretic separation device (MSD) connected to the one or more incubation tubings, wherein the MSD applies an exponentially increasing magnetophoretic gradient to capture the magnetically labeled non-target cells; and
(vi) one or more outlets connected to the MSD, wherein the one or more types of target cells are expelled from the one or more outlets; and
b) a processor programmed to control the microfluidic magnetic device according to the computer implemented method of claim 84.

87. A computer implemented method for determining positioning of one or more fluidic conduits and one or more magnetic flux concentrators (MFC) relative to the plurality of magnets of the magnetophoretic separation device (MSD) of any one of claims 1-42, the computer performing steps comprising:
modeling magnetic field and magnetophoretic forces at different locations relative to a magnetic array; and
identifying one or more sets of spatial coordinates to position one or more fluidic conduits and one or more magnetic flux concentrators relative to the plurality of magnets to achieve a target exponential magnetophoretic force profile along the fluidic conduit.

88. The computer implemented method of claim 87, wherein the computer performs steps comprising:
a) generating a target mathematical profile of max ∥(∇·\vec{B})\vec{B}∥, or another metric for a magnetic force field, as a function of the path of the fluidic conduit smax ∥(∇·\vec{B})\vec{B}∥ [symbol] metric is set according to the highest value omax ∥(∇·\vec{B})\vec{B}∥ [symbol] ;
b) identifying different combinations of zₚₐₜₕ and d_{MFC} that could generate a target magnetophoretic force profile along the path of the fluidic conduit by plotting max ∥(∇·\vec{B})\vec{B}∥ obtained from a parametric sweep on a surface as a function of zₚₐₜₕ and d_{MFC}, and where zₚₐₜₕ can be defined in any direction relative to the magnetic array, magnetic flux concentrator, or any other logical definition;
c) identifying candidate paths (q) on the max ∥(∇·\vec{B})\vec{B}∥ surface that would generate a target profile f(s);
d) translating the candidate parametric paths into physical path positions;
e) performing a high-resolution numerical simulation of the magnetic field in 3-dimensional (3D) free space over the magnetic array to identify a set of (x, y, z) points in the simulation that satisfy the following two criteria: 1) d\vec{B}(x, y, z)/dy≈0, and 2) ∥\vec{B}_{3D,yz}(x,y,z)∥≈∥\vec{B}_{2D}(zₚₐₜₕ)∥;
f) displaying sets of 3D candidate paths translated from parameter space that achieve the target magnetophoretic force profile;
g) evaluating 3D candidate paths using a cost function to select for a path that minimizes the following functions:
$${α}_{1} = max ( \frac{dz}{dx} ) - 1 , \qquad 1 )$$
which selects for paths with gradual slopes for the fluidic conduit to follow in the xz-plane,
$${α}_{2} = {\int }_{0}^{L} {d}_{MFC} dx - \frac{0.45 L}{2} , \qquad 2 )$$
which selects for MFC paths with gradually varying d_{MFC} from 450 to 0 μm across the total length L of the path to facilitate the 3D printing and the threading of the MFC. 3) Σⱼ₌₁^{N}|∥\vec{B}_{2D,j}∥−∥\vec{B}_{3D,yz,j}∥|, which selects for paths that can best match ∥\vec{B}_{3D,yz}(x, y, z)∥ with ∥\vec{B}_{2D}(zₚₐₜₕ)∥ across all N points in the path; and
h) selecting the path e with the minimum cost function
$${min}_{θ} \frac{1}{3} {\sum }_{i = 1}^{3} {[ {α}_{i} ( θ ) ]}^{2}$$
as the final position of the fluidic conduit and the MFC to construct the MSD.

89. A computer implemented method for determining positioning of a fluidic conduit and a magnetic flux concentrator (MFC) relative to the plurality of magnets of the magnetophoretic separation device (MSD) of any one of claims 1-42, the computer performing steps comprising:
modeling magnetic field and magnetophoretic forces at different locations relative to the magnetic array; and
modeling fluidic forces on the cells and the magnetic nanoparticles present within the channel; and
identifying a set of spatial coordinates to position the fluidic conduit and the magnetic
flux concentrator relative to the plurality of magnets to achieve a target captured cell density along the fluidic conduit.

90. The computer implemented method of claim 89, wherein the computer performs steps comprising:
a) generating a target mathematical cell capture profile for capture of magnetically labeled non-target cells;
b) performing a high-resolution numerical simulation of the magnetic field in 3-dimensional (3D) free space over the magnet array for a variety of values of the MFC dimensions to identify the magnetic force F=η(B·grad(B)) on the magnetically labeled non-target cells relative to the magnet array, where η is a constant which comprises the number of magnetic particles attached to a cell, the volume of the magnetic particles and the difference in susceptibility between the magnetic particles and the fluid;
c) performing a high-resolution numerical simulation of the fluid flow in 3-dimensional (3D) free space within the MSD channel to determine the fluidic force on cells for a given flow rate;
d) performing particle tracking simulations of cells within the MSD channel based on the sum of both fluidic force (c) and magnetic force (b); and
e) using the particle simulations to predict final cell density on the channel wall and iterating or performing an optimization on the MFC position to achieve the desired cell capture profile in (a)

91. The computer implemented method of claim 90, wherein said performing step b) comprises performing said high-resolution numerical simulation using a variety of values of the dimensions of the gap from the MFC to the channel.

92. The computer implemented method of any one of claims 87-91, wherein the fluidic conduit is provided by fluidic tubing positioned adjacent to the magnetic array or the magnetic flux concentrator.

93. A non-transitory computer-readable medium comprising program instructions that, when executed by a processor in a computer, causes the processor to perform the method of any one of claims 87-92.
